(19) United States
(12) Patent Application Publication
(10) Pub. No.: US 2022/0054545 A1
(43) Pub. Date: Feb. 24, 2022

(54) ANTI-GPC3 CHIMERIC ANTIGEN RECEPTORS (CARs) IN COMBINATION WITH TRANS CO-STIMULATORY MOLECULES AND THERAPEUTIC USES THEREOF

(71) Applicant: Sotio, LLC, Boston, MA (US)

(72) Inventors: Kathleen McGinness, Cambridge, MA (US); Charles Wilson, Cambridge, MA (US); Brant Herrin, Cambridge, MA (US); Gregory Motz, Cambridge, MA (US)

(73) Assignee: Sotio, LLC, Boston, MA (US)

(21) Appl. No.: 17/291,963

(22) PCT Filed: Nov. 7, 2019
(86) PCT No.: PCT/US2019/060287

§ 371 Date: May 6, 2021

Related U.S. Application Data
(60) Provisional application No. 62/756,683, filed on Nov. 7, 2018.

(51) Int. Cl. A61K 35/17; C07K 14/725; C07K 14/47; C07K 14/705; A61P 35/00

(52) U.S. Cl. CPC A61K 35/17; C07K 14/7051; A61P 35/00; C07K 14/70521; C07K 14/70575; C07K 14/4725

(57) ABSTRACT

Disclosed herein are genetically engineered hematopoietic cells (e.g., genetically engineered hematopoietic stem cells, or genetically engineered immune cells), which co-express one or more co-stimulatory polypeptides with an anti-GPC3 chimeric antigen receptor (CAR), and uses thereof for enhancing T cell anti-tumor activity in a subject in need of the treatment.

### RELATED APPLICATIONS

This application is a United States National Phase Application under 35 U.S.C. § 371 of International Application No. PCT/US2019/060287, filed Nov. 7, 2021, which claims the benefit of the filing date of U.S. Provisional Application No. 62/756,683, filed Nov. 7, 2018. The entire contents of the prior applications are incorporated by reference herein.

### SEQUENCE LISTING

The application contains a Sequence Listing that has been filed electronically in the form of a text file, created May 6, 2021, and named “112309-0089-70010US01_SEQ.TXT” (186,729 bytes), the contents of which are incorporated by reference herein in their entirety.

### BACKGROUND OF DISCLOSURE

Cancer immunotherapy, including cell-based therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues.

Cell-based therapy may involve cytotoxic T cells having reactivity skewed toward cancer cells. Eshhar et al., Proc. Natl. Acad. Sci. U.S.A; 1993; 90(2):720-724; Geiger et al., J Immunol. 1999; 162(10):5931-5939; Brentjens et al., Nat. Med. 2003; 9(3):279-286; Cooper et al., Blood. 2003; 101(4):1637-1644; and Imai et al., Leukemia. 2004; 18:676-684. One approach is to express a chimeric receptor having an antigen-binding domain fused to one or more T cell activation signaling domains. Binding of a cancer antigen via the antigen-binding domain results in T cell activation and triggers cytotoxicity. Efficacy of chimeric receptor-expressing autologous T lymphocytes in treating B-cell precursor acute lymphoblastic leukemia (ALL) has been demonstrated in clinical trials. Pule et al., Nat. Med. 2008; 14(11):1264-1270; Porter et al., N Engl J Med; 2011; 25;365(8):725-733; Brentjens et al., Blood. 2011; 118(18):4817-4828; Till et al., Blood. 2012; 119(17):3940-3950; Kochenderfer et al., Blood. 2012; 119(12):2709-2720; and Brentjens et al., Sci Transl Med. 2013;5(177):177ra138.

It is of great interest to develop new strategies to enhance efficacy of cell-based immune therapies.

### SUMMARY OF DISCLOSURE

The present disclosure is based on the development of strategies to co-express a co-stimulatory polypeptide and an anti-GPC3 chimeric antigen receptor (CAR) for use in cell-based immune therapy (i.e., expressing two separate polypeptides). Modulation of costimulatory pathways may be achieved by expressing (e.g., over-expressing) in hematopoietic cells (e.g., hematopoietic stem cells, immune cells, such as T cells or natural killer cells) one or more co-stimulatory polypeptides such as those described herein. In some instances, hematopoietic cells that co-express one or more co-stimulatory polypeptides and an anti-GPC3 CAR would be expected to exhibit superior bioactivities, for example, cell proliferation, activation (e.g., increased cytokine production, e.g., IL-2 or IFN-γ production), cytotoxicity, and/or in vivo anti-tumor activity.

Accordingly, provided herein are modified (e.g., genetically modified) hematopoietic cells (e.g., hematopoietic stem cells, immune cells, such as T cells or natural killer cells) that have the capacity for modulation of costimulatory pathways relative to the wild-type hematopoietic cells of the same type. In some instances, the modified hematopoietic cells may express or overly express a co-stimulatory polypeptide. The co-stimulatory polypeptide may be a member of the B7/CD28 superfamily, a member of the tumor necrosis factor (TNF) superfamily, or a ligand thereof. Exemplary members of the B7/CD28 superfamily or ligands thereof include, but are not limited to, CD28, CD80, CD86, ICOS, ICOSL, B7-H3, B7-H4, VISTA, TMIGD2, B7-H6, B7-H7, and variants thereof. Exemplary members of the TNF superfamily or ligands thereof include, but are not limited to, 4-1BB, 4-1BBL, BAFF, BAFFR, CD27, CD70, CD30, CD30L, CD40, CD40L, DR3, GITR, GITRL, HVEM, LIGHT, TNF-beta, OX40, OX40L, RELT, TACI, TL1A, TNF-alpha, and TNFRII. Additional examples include BCMA, EDAR2, TROY, LTBR, EDAR, NGFR, OPG, RANK, DCR3, TNFR1, FN14 (TweakR), APRIL, EDA-A2, TWEAK, LTb (TNF-C), NGF, EDA-A1, amyloid precursor protein (APP), TRAIL.

In some embodiments, the member of the B7/CD28 superfamily, member of the tumor necrosis factor (TNF) superfamily, or ligand thereof is a wild type sequence. In some embodiments, the member of the B7/CD28 superfamily, member of the tumor necrosis factor (TNF) superfamily, or ligand thereof is a variant sequence (i.e., comprising one or more insertions, deletions, or mutations in comparison with a wild type sequence). For example, the 4-1BBL may be 4-1BBL Q89A, 4-1BBL L115A, 4-1BBL K127A, or 4-1BBL Q227A. In some embodiments, the member of the B7/CD28 superfamily, member of the tumor necrosis factor (TNF) superfamily, or ligand thereof may lack a cytoplasmic domain. In an exemplary embodiment, the 4-1BBL lacks a cytoplasmic domain. In some embodiments, the member of the TNF superfamily or ligand thereof is not 4-1BBL.

In some embodiments, the co-stimulatory polypeptide co-expressed with any of the anti-GPC3 CARs described herein is free of any F506 binding protein (FKBP) such as FKBPv36. In some examples, the co-stimulatory polypeptide is free of a signaling domain derived from MyD88.

The modified hematopoietic cells may further express an anti-GPC3 CAR, which may comprise (a) an extracellular antigen binding domain, wherein the extracellular-binding domain binds GPC3; (b) a transmembrane domain; and (c) a cytoplasmic signaling domain. In some examples, (c) is located at the C-terminus of the anti-GPC3 CAR. In some instances, the anti-GPC3 CAR may further comprise at least one co-stimulatory signaling domain. In other instances, the anti-GPC3 CAR may be free of co-stimulatory signaling domains.

In some examples, the extracellular antigen binding domain of (a) is a single chain antibody fragment that is specific to (i.e., binds to) GPC3.

In some embodiments, the transmembrane domain of (b) in any of the CAR polypeptides can be of a single-pass membrane protein, e.g., CD8α, CD8β, 4-1BB, CD28, CD34, CD4, FcεRIγ, CD16A, OX40, CD3ζ, CD3ε, CD3γ, CD3δ, TCRα, CD32, CD64, VEGFR2, FAS, and FGFR2B. Alternatively, the transmembrane domain of (b) can be a non-naturally occurring hydrophobic protein segment.

In some embodiments, the at least one co-stimulatory signaling domain of the CAR polypeptides described herein, if applicable, can be of a co-stimulatory molecule, which can be 4-1BB, CD28, CD28_{LL→GG} variant, OX40, ICOS, CD27, GITR, ICOS, HVEM, TIM1, LFA1, and CD2. In some examples, the at least one co-stimulatory signaling domain is a CD28 co-stimulatory signaling domain or a 4-1BB co-stimulatory signaling domain. In some instances, the CAR polypeptide may comprise two co-stimulatory signaling domains. In some instances, one of the co-stimulatory signaling domains is a CD28 co-stimulatory signaling domain; and the other co-stimulatory domain can be a 4-1BB co-stimulatory signaling domain, an OX40 co-stimulatory signaling domain, a CD27 co-stimulatory signaling domain, or an ICOS co-stimulatory signaling domain. Specific examples include, but are not limited to, CD28 and 4-1BB; or CD28_{LL→GG} variant and 4-1BB.

In some embodiments, the cytoplasmic signaling domain of (c) in any of the CAR polypeptides described herein can be a cytoplasmic domain of CD3ζ or FcεR1γ.

In some embodiments, the hinge domain of any of the CAR polypeptides described herein, when applicable, can be of CD28, CD16A, CD8α, or IgG. In other examples, the hinge domain is a non-naturally occurring peptide. For example, the non-naturally occurring peptide may be an extended recombinant polypeptide (XTEN) or a (Gly₄Ser)ₙ polypeptide, in which n is an integer of 3-12, inclusive. In some examples, the hinge domain is a short segment, which may contain up to 60 amino acid residues.

In specific examples, the CAR polypeptide comprises (i) a CD28 co-stimulatory domain or a 4-1BB co-stimulatory domain; and (ii) a CD28 transmembrane domain, a CD28 hinge domain, or a combination thereof. In some embodiments, the CAR polypeptide comprises (i) a CD28 co-stimulatory domain or a 4-1BB co-stimulatory domain; and (ii) a CD8 transmembrane domain, a CD8 hinge domain, or a combination thereof. For example, the CAR polypeptide may comprise the amino acid sequence of SEQ ID NO: 1 or SEQ ID NO: 2.

In some embodiments, the genetically engineered hematopoietic cells co-express a CAR polypeptide and a co-stimulatory polypeptide. In some embodiments, the CAR polypeptide comprises a co-stimulatory domain of a CD28 co-stimulatory molecule, and the co-stimulatory polypeptide is BAFFR or CD27. In some embodiments, the CAR polypeptide comprises a co-stimulatory domain of a CD28 co-stimulatory molecule, and the co-stimulatory polypeptide is BAFFR. In some embodiments, the CAR polypeptide comprises a co-stimulatory domain of a CD28 co-stimulatory molecule, and the co-stimulatory polypeptide is CD27. The CD28 co-stimulatory molecule may comprise the amino acid sequence of SEQ ID NO: 12. The BAFFR may comprise the amino acid sequence of SEQ ID NO: 62, and the CD27 may comprise the amino acid sequence of SEQ ID NO: 33. In other embodiments, the CAR polypeptide comprises a co-stimulatory domain of a 4-1BB co-stimulatory molecule, and the co-stimulatory polypeptide is CD70, LIGHT, or OX40L. The 4-1BB co-stimulatory molecule may comprise the amino acid sequence of SEQ ID NO: 22. The CD70 may comprise the amino acid sequence of SEQ ID NO: 34, the LIGHT may comprise the amino acid sequence of SEQ ID NO: 43, and the OX40L may comprise the amino acid sequence of SEQ ID NO: 47.

The hematopoietic cells described herein, expressing the co-stimulatory polypeptide and anti-GPC3 CAR, may be a hematopoietic stem cell or a progeny thereof. In some embodiments, the hematopoietic cells can be immune cells such as natural killer cell, monocyte/macrophage, neutrophil, eosinophil, or T cell. The immune cells can be derived from peripheral blood mononuclear cells (PBMC), hematopoietic stem cells (HSCs), or induced pluripotent stem cells (iPSCs). In some examples, the immune cell is a T cell, in which the expression of an endogenous T cell receptor, an endogenous major histocompatibility complex, an endogenous beta-2-microglobulin, or a combination thereof has been inhibited or eliminated.

Any of the hematopoietic cells described herein may comprise a nucleic acid or a nucleic acid set, which collectively comprises: (a) a first nucleotide sequence encoding the co-stimulatory polypeptide; and (b) a second nucleotide sequence encoding the CAR polypeptide. In some embodiments, the nucleic acid or the nucleic acid set is an RNA molecule or a set of RNA molecules. In some instances, the immune cell comprises the nucleic acid, which comprises both the first nucleotide sequence and the second nucleotide sequence. In some embodiments, the coding sequence of the co-stimulatory polypeptide is upstream of that of the CAR polypeptide. In some embodiments, the coding sequence of the CAR polypeptide is upstream of that of the co-stimulatory polypeptide. Such a nucleic acid may further comprise a third nucleotide sequence located between the first nucleotide sequence and the second nucleotide sequence, wherein the third nucleotide sequence encodes a ribosomal skipping site (e.g., a P2A peptide), an internal ribosome entry site (IRES), or a second promoter.

In some examples, the nucleic acid or the nucleic acid set is comprised within a vector or a set of vectors, which can be an expression vector or a set of expression vectors (e.g., viral vectors such as a retroviral vector, which is optionally a lentiviral vector or a gammaretroviral vector). A nucleic acid set or a vector set refers to a group of two or more nucleic acid molecules or two or more vectors, each encoding one of the polypeptides of interest (i.e., the co-stimulatory polypeptide and the CAR polypeptide). Any of the nucleic acids described herein is also within the scope of the present disclosure.

In another aspect, the present disclosure provides a pharmaceutical composition, comprising any of the hematopoietic cells described herein, and a pharmaceutically acceptable carrier.

Moreover, provided herein is a method for inhibiting cells expressing GPC3 (e.g., reducing the number of such cells, blocking cell proliferation, and/or suppressing cell activity) in a subject, the method comprising administering to a subject in need thereof a population of the hematopoietic cells described herein, which may co-express the co-stimulatory polypeptide and the CAR polypeptide, and/or the pharmaceutical composition described herein.

In some examples, the hematopoietic cells are autologous. In other examples, the hematopoietic cells are allogeneic. In any of the methods described herein, the hematopoietic cells can be activated, expanded, or both ex vivo. In some instances, the hematopoietic cells comprise immune cells comprising T cells, which are activated in the presence of one or more of anti-CD3 antibody, anti-CD28 antibody, IL-2, phytohemagglutinin, and an engineered artificial stimulatory cell or particle. In other instances, the immune cells comprise natural killer cells, which are activated in the presence of one or more of 4-1BB ligand, anti-4-1BB antibody, IL-15, anti-IL-15 receptor antibody, IL-2, IL-12, IL-18, IL-21, K562 cells, and an engineered artificial stimulatory cell or particle.

In some examples, the subject to be treated by the methods described herein may be a human patient suffering from a cancer. Specific non-limiting examples of cancers which can be treated by the methods of the disclosure include, for example, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1C include a series of graphs showing the fold expansion of T cells relative to the previous time point for T cells after stimulation with GPC3-expressing Hep3B cells. The T cells evaluated in this experiment expressed anti-GPC3 CAR with a 4-1BB costimulatory domain (SEQ ID NO: 1) alone (FIGS. 1A-1C) or in combination with CD70 (FIG. 1A; SEQ ID NO: 34), LIGHT (FIG. 1B; SEQ ID NO: 43), or OX40L (FIG. 1C; SEQ ID NO: 47), or anti-GPC3 CAR with a CD28 costimulatory domain (SEQ ID NO: 2) alone (A, B, and C) or in combination with CD70 (FIG. 1A; SEQ ID NO: 34), LIGHT (FIG. 1B; SEQ ID NO: 43), or OX40L (FIG. 1C; SEQ ID NO: 47).

FIGS. 2A-2D include a series of graphs showing the fold expansion of T cells relative to the previous time point for T cells after stimulation with GPC3-expressing JHH7 cells as a function of stimulation round (FIG. 2A) and cytokine production after the second round of stimulation for IL-2 (FIG. 2B), IFN-gamma (FIG. 2C), and IL-17A (FIG. 2D). Data are shown for T cells expressing anti-GPC3 CAR with a 4-1BB costimulatory domain (SEQ ID NO: 1) alone or in combination with CD70 (SEQ ID NO: 34), LIGHT (SEQ ID NO: 43), or OX40L (SEQ ID NO: 47).

FIGS. 3A and 3B include a series of graphs showing enhanced IL-2 production (FIG. 3A) and proliferation (FIG. 3B) for T cells expressing an anti-GPC3 CAR polypeptide with a 4-1BB costimulatory domain (GPC3-CAR-4-1BB; SEQ ID NO: 1) and T cells co-expressing GPC3-CAR-4-1BB and CD70 (SEQ ID NO: 34), LIGHT (SEQ ID NO: 43), or OX40L (SEQ ID NO: 47).

FIGS. 4A-4C include a series of graphs demonstrating function of T cells expressing an anti-GPC3 CAR polypeptide with a 4-1BB costimulatory domain (GPC3-CAR-4-1BB; SEQ ID NO: 1) or GPC3-CAR-4-1BB in combination with CD70 (SEQ ID NO: 34), LIGHT (SEQ ID NO: 43), or OX40L (SEQ ID NO: 47). T cells were evaluated for their ability to produce IL-17A (FIG. 4A) and proliferate (FIG. 4B) under chronic stimulation. Additionally, T cells were evaluated for their ability to proliferate after a single stimulation (FIG. 4C).

FIGS. 5A-5D include a series of graphs demonstrating function of T cells expressing an anti-GPC3 CAR polypeptide with a CD28 costimulatory domain (GPC3-CAR-CD28; SEQ ID NO: 2) or GPC3-CAR-CD28 in combination with CD27 (SEQ ID NO: 33). T cells were evaluated for their ability to proliferate (FIGS. 5A and 5B) and generate cytokines (FIGS. 5C and 5D).

FIGS. 6A and 6B include a series of graphs demonstrating function of T cells expressing an anti-GPC3 CAR polypeptide with a CD28 costimulatory domain (GPC3-CAR-CD28; SEQ ID NO: 2) or GPC3-CAR-CD28 in combination with CD27 (SEQ ID NO: 33). T cells were evaluated for their ability to proliferate in the presence of immunosuppressive myeloid-derived suppressor cells (MDSCs; FIG. 6A) or regulatory T cells (Tregs; FIG. 6B).

FIGS. 7A-7C include a series of graphs showing anti-tumor activity of T cells expressing an anti-GPC3 CAR polypeptide with a 4-1BB costimulatory domain (GPC3-CAR-4-1BB; SEQ ID NO: 1) or GPC3-CAR-4-1BB in combination with CD70 (SEQ ID NO: 34), LIGHT (SEQ ID NO: 43), or OX40L (SEQ ID NO: 47). HepG2 (FIG. 7A), Hep3B (FIG. 7B), and JHH7 (FIG. 7C) tumor xenograft models were evaluated in NSG mice.

FIG. 8 is a graph showing anti-tumor activity of T cells expressing an anti-GPC3 CAR polypeptide with a CD28 costimulatory domain (GPC3-CAR-CD28; SEQ ID NO: 2) or GPC3-CAR-CD28 in combination with CD27 (SEQ ID NO: 33) in a JHH7 tumor xenograft models in NSG mice.

FIGS. 9A and 9B include a series of graphs showing the amount of T cells in mouse blood from HepG2 (panel A) and Hep3B (panel B) tumor xenograft models in NSG mice. Data are shown for T cells expressing anti-GPC3 CAR with a 4-1BB costimulatory domain (SEQ ID NO: 1) alone or in combination with CD70 (SEQ ID NO: 34) (FIG. 9A) and T cells expressing anti-GPC3 CAR with a CD28 costimulatory domain (SEQ ID NO: 2) alone or in combination with CD27 (SEQ ID NO: 33) (FIG. 9B).

FIGS. 10A-10D include a series of graphs showing CD70 expression on T cells expressing anti-GPC3 CAR with a 4-1BB costimulatory domain (SEQ ID NO: 1) alone or in combination with CD70 (SEQ ID NO: 34) (FIGS. 10A and 10B) or CD27 expression on T cells expressing anti-GPC3 CAR with a CD28 costimulatory domain (SEQ ID NO: 2) alone or in combination with CD27 (SEQ ID NO: 33) (FIGS. 10C and 10D).

### DETAILED DESCRIPTION OF DISCLOSURE

Chimeric antigen receptors (CARs) are artificial cell-surface receptors that redirect binding specificity of immune cells (e.g., T cells) expressing such to diseased cells such as cancer cells, thereby eliminating the target disease cells via, e.g., the effector activity of the immune cells. A CAR construct often comprises an extracellular antigen binding domain fused to at least an intracellular signaling domain. The extracellular antigen binding domain (e.g., a single-chain antibody fragment) is specific to an antigen of interest (e.g., a tumor antigen) and the intracellular signaling domain can mediate a cell signaling that lead to activation of immune cells. As such, immune cells expressing a CAR construct can bind to diseased cells (e.g., tumor cells) expressing the target antigen, leading to activation of the immune cells and elimination of the diseased cells.

The present disclosure is based, at least in part, on the development of strategies for enhancing activities of effector immune cells that co-express an anti-glypican-3 (GPC3) chimeric antigen receptor (CAR) polypeptide. In particular, the present disclosure features methods for imparting the capacity to modulate suitable co-stimulatory pathways by the effector immune cells, thereby enhancing their growth and bioactivity. For example, T cells co-expressing an anti-GPC3 CAR comprising a 4-1BB co-stimulatory domain and certain co-stimulatory molecules (e.g., CD70, LIGHT, and OX40L) and T cells co-expressing an anti-GPC3 CAR comprising a CD28 co-stimulatory domain and certain co-stimulatory molecules (e.g., CD27) showed enhanced cell proliferation and cytokine production. The immunosuppressive features within solid tumors may limit the success of engineered T cell therapies. The approach disclosed herein, involving the co-expression of an anti-GPC3 CAR and a co-stimulatory polypeptide (which provides a co-stimulation signal in trans), aims at, at least in part, overcoming this key challenge in tumor treatment, particularly solid tumor treatment.

In some instances, the capacity of the effector immune cells to modulate co-stimulatory pathways may be observed in normal cellular environments. In other instances, the capacity of the effector immune cells to modulate co-stimulatory pathways may be observed under conditions that may be found in a tumor microenvironment. The present disclosure provides various approaches to modulate (e.g., to stimulate) co-stimulatory pathways including by, e.g., expressing or overexpressing co-stimulatory polypeptides. The co-stimulatory polypeptides for use in the present disclosure may be members of the B7/CD28 superfamily, members of the tumor necrosis factor (TNF) superfamily or ligands thereof that functional as a co-stimulatory factor in one or more types of immune cells. A co-stimulatory factor refers to a receptor or a ligand thereof, which enhances the primary, antigen-specific signal and fully activates immune cells.

Accordingly, the present disclosure provides modified (e.g., genetically engineered) hematopoietic cells (e.g., hematopoietic stem cells, immune cells, such as T cells or natural killer cells) that have the capacity to have modulated (e.g., increased) co-stimulatory pathways. In some embodiments, such a modified hematopoietic cell may express one or more co-stimulatory polypeptides such as those described herein to impart the capacity to modulate the co-stimulatory pathways, relative to an unmodified hematopoietic cell. Such a genetically engineered hematopoietic cell may further express a CAR polypeptide (as a separate polypeptide relative to the co-stimulatory polypeptide). Both the CAR polypeptide and the co-stimulatory polypeptide expressed in the genetically engineered hematopoietic cells are encoded by nucleic acids exogenous to the immune cells (i.e., introduced into immune cells via recombinant technology). They are not encoded by endogenous genes of the hematopoietic cells absent of the involved genetic engineering. The present disclosure also provides pharmaceutical compositions and kits comprising the described genetically engineered hematopoietic cells.

The genetically engineered hematopoietic cells described herein, expressing (e.g., overexpressing) a co-stimulatory peptide, may confer at least the following advantages. The expression of the co-stimulatory polypeptide would have the capacity to modulate the co-stimulatory pathways. As such, the genetically engineered hematopoietic cells may proliferate better, produce more cytokines, exhibit greater anti-tumor cytotoxicity, and/or exhibit greater T cell survival relative to hematopoietic cells that do not express (or do not over-express) the co-stimulatory polypeptide, leading to enhanced cytokine production, survival rate, cytotoxicity, and/or anti-tumor activity.

I. Co-Stimulatory Polypeptides

As used herein, a co-stimulatory polypeptide refers to a polypeptide that has the capacity to modulate (e.g., stimulate) a co-stimulatory pathway. Such a polypeptide may modulate (e.g., increase) the co-stimulatory pathway via any mechanism. In some examples, the co-stimulatory polypeptide may comprise a co-stimulatory receptor or the co-stimulatory signaling domain thereof. In other examples, the co-stimulatory polypeptide may comprise a ligand of a co-stimulatory receptor or a signaling domain thereof where applicable. Such a ligand may trigger a co-stimulatory signaling pathway upon binding to the cognate co-stimulatory receptor. Alternatively, the co-stimulatory polypeptide may be a non-naturally occurring polypeptide that mimics the activity of a naturally-occurring ligand to any of the co-stimulatory receptors disclosed herein. Such a non-naturally occurring polypeptide may be a single-chain agonistic antibody specific to a co-stimulatory receptor, e.g., an scFv specific to 4-1BB and mimics the activity of 4-1BBL.

Exemplary co-stimulatory polypeptides may include, but are not limited to, members of the B7/CD28 superfamily, members of the tumor necrosis factor (TNF) superfamily or ligands thereof (e.g., CD28, CD80, CD86, ICOS, ICOSL, B7-H3, B7-H4, VISTA, TMIGD2, B7-H6, B7-H7, 4-1BB, 4-1BBL, BAFF, BAH-R, CD27, CD70, CD30, CD30L, CD40, CD40L, DR3, GITR, GITRL, HVEM, LIGHT, TNF-beta, OX40, OX40L, RELT, TALI, TL1A, TNF-alpha, or TNFRII). Additional examples include BCMA, EDAR2, TROY, LTBR, EDAR, NGFR, OPG, RANK, DCR3, TNFR1, FN14 (TweakR), APRIL, EDA-A2, TWEAK, LTb (TNF-C), NGF, EDA-A1, amyloid precursor protein (APP), TRAIL. Any such polypeptide from any suitable species (e.g., a mammal such as a human) may be contemplated for use with the compositions and methods described herein. In some embodiments, the co-stimulatory polypeptides do not comprise the combination of CD40 and MyD88.

As used herein, a co-stimulatory polypeptide that is a member of the B7/CD28 superfamily or a member of the TNF superfamily refers to a member of either superfamily that plays co-stimulatory roles in activation of any type of immune cells. Such a member may be a naturally-occurring receptor or ligand of either superfamily. Alternatively, such a member may be a variant of the naturally-occurring receptor or ligand. The variant may have increased or decreased activity relative to the native counterpart. In some examples, the variant lacks the cytoplasmic domain or a portion thereof relative to the native counterpart. Described below are exemplary co-stimulatory polypeptides that can be used in the present disclosure.

CD28 (Cluster of Differentiation 28) is a protein expressed on T cells that provides co-stimulatory signals required for T cell activation and survival. It is the receptor for CD80 and CD86 proteins, and is the only B7 receptor constitutively expressed on naïve T cells. The amino acid sequence of an exemplary human CD28 is provided below:

CD28
(SEQ ID NO: 12)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

CD80 (Cluster of Differentiation 80; B7-1) is a protein found on dendritic cells, activated B cells, and monocytes. It provides a co-stimulatory signal necessary for T cell activation and survival. CD80 is a ligand of both CD28 and CTLA-4. The amino acid sequence of an exemplary human CD80 is provided below:

CD80
(SEQ ID NO: 13)
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFAPRCRERRRNERLRRESVRPV

CD86 (Cluster of Differentiation 86; B7-2) is a type I membrane protein that is a member of the immunoglobulin superfamily CD86 is expressed on antigen-presenting cells that provide co-stimulatory signals necessary for T cell activation and survival. CD86 is a ligand of both CD28 and CTLA-4. The amino acid sequence of an exemplary human CD86 is provided below:

CD86
(SEQ ID NO: 14)
MDPQCTMGLSNILFVMAELLSGAAPLKIQAYFNETADLPCQFANSQNQSL
SELVVFWQDQENLVLNEVYLGKEKFDSVHSKYMGRTSFDSDSWTLRLHNL
QIKDKGLYQCIIHHKKPTGMIRIHQMNSELSVLANFSQPEIVPISNITEN
VYINLTCSSIHGYPEPKKMSVLLRTKNSTIEYDGVMQKSQDNVTELYDVS
ISLSVSFPDVTSNMTIFCILETDKTRLLSSPFSIELEDPQPPPDHIPWIT
AVLPTVIICVMVFCLILWKWKKKKRPRNSYKCGTNTMEREESEQTKKREK
IHIPERSDEAQRVEKSSKTSSCDKSDTCF

ICOS (CD278; Inducible T cell co-stimulator; or CVID1) is a member of the CD28-superfamily ICOS is expressed on activated T cells. The amino acid sequence of an exemplary human ICOS is provided below:

ICOS
(SEQ ID NO: 15)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL

ICOSL (ICOSLG; B7-H2; CD275) is a protein that is a ligand for T cell specific protein ICOS. ICOSL acts as a co-stimulatory signal for T cell proliferation and cytokine secretion. The amino acid sequence of an exemplary human ICOSL is provided below:

ICOSL
(SEQ ID NO: 16)
MRLGSPGLLFLLFSSLRADTQEKEVRAMVGSDVELSCACPEGSRFDLNDV
YVYWQTSESKTVVTYHIPQNSSLENVDSRYRNRALMSPAGMLRGDFSLRL
FNVTPQDEQKFHCLVLSQSLGFQEVLSVEVTLHVAANFSVPVVSAPHSPS
QDELTFTCTSINGYPRPNVYWINKTDNSLLDQALQNDTVFLNMRGLYDVV
SVLRIARTPSVNIGCCIENVLLQQNLTVGSQTGNDIGERDKITENPVSTG
EKNAATWSILAVLCLLVVVAVAIGWVCRDRCLQHSYAGAWAVSPETELTG
HV

B7-H3 (CD276; Cluster of Differentiation 276) is a member of the immunoglobulin superfamily that is thought to participate in the regulation of T cell-mediated immune response. The amino acid sequence of an exemplary human B7-H3 is provided below:

B7-H3
(SEQ ID NO: 17)
MLRRRGSPGMGVHVGAALGALWFCLTGALEVQVPEDPVVALVGTDATLCC
SFSPEPGESLAQLNLIWQLTDTKQLVHSFAEGQDQGSAYANRTALFPDLL
AQGNASLRLQRVRVADEGSFTCFVSIRDFGSAAVSLQVAAPYSKPSMTLE
PNKDLRPGDTVTITCSSYQGYPEAEVFWQDGQGVPLTGNVTTSQMANEQG
LFDVHSILRVVLGANGTYSCLVRNPVLQQDAHSSVTITPQRSPTGAVEVQ
VPEDPVVALVGTDATLRCSFSPEPGFSLAQLNLIWQLTDTKQLVHSFTEG
RDQGSAYANRTALFPDLLAQGNASLRLQRVRVADEGSFTCFVSIRDFGSA
AVSLQVAAPYSKPSMTLEPNKDLRPGDTVTITCSSYRGYPEAEVFWQDGQ
GVPLTGNVTTSQMANEQGLFDVHSVLRVVLGANGTYSCLVRNPVLQQDAH
GSVTITGQPMTFPPEALWVTVGLSVCLIALLVALAFVCWRKIKQSCEEEN
AGAEDQDGEGEGSKTALQPLKHSDSKEDDGQEIA

VISTA (V-domain Ig suppressor of T cell activation; B7-H5; PD-1H) is a Type I transmembrane protein that functions as an immune checkpoint. VISTA co-stimulates T cells via TMIGD2 (CD28H). The amino acid sequence of an exemplary human VISTA is provided below:

VISTA
(SEQ ID NO: 18)
MGVPTALEAGSWRWGSLLFALFLAASLGPVAAFKVATPYSLYVCPEGQNV
TLTCRLLGPVDKGHDVTFYKTWYRSSRGEVQTCSERRPIRNLTFQDLHLH
HGGHQAANTSHDLAQRHGLESASDHHGNFSITMRNLTLLDSGLYCCLVVE
IRHHHSEHRVHGAMELQVQTGKDAPSNCVVYPSSSQDSENITAAALATGA
CIVGILCLPLILLLVYKQRQAASNRRAQELVRMDSNIQGIENPGFEASPP
AQGIPEAKVRHPLSYVAQRQPSESGRHLLSEPSTPLSPPGPGDVFFPSLD
PVPDSPNFEVI

TMIGD2 (Transmembrane and immunoglobulin domain containing 2; CD28H) is a TMIGD2 is thought to enhance T cell proliferation and cytokine production via an AKT-dependent signaling cascade. The amino acid sequence of an exemplary human TMIGD2 is provided below:

TMIGD2
(SEQ ID NO: 19)
MGSPGMVLGLLVQIWALQEASSLSVQQGPNLLQVRQGSQATLVCQVDQAT
AWERLRVKWTKDGAILCQPYITNGSLSLGVCGPQGRLSWQAPSHLTLQLD
PVSLNHSGAYVCWAAVEIPELEEAEGNITRLFVDPDDPTQNRNRIASFPG
FLEVLLGVGSMGVAAIVWGAWFWGRRSCQQRDSGNSPGNAFYSNVLYRPR
GAPKKSEDCSGEGKDQRGQSIYSTSFPQPAPRQPHLASRPCPSPRPCPSP
RPGHPVSMVRVSPRPSPTQQPRPKGFPKVGEE

B7-H6 (NCR3LG1; Natural Killer Cell Cytotoxicity Receptor 3 Ligand 1) is a member of the B7 family selectively expressed on tumor cells. B7-H6 interacts with NKp30, resulting in natural killer (NK) cell activation and cytotoxicity. The amino acid sequence of an exemplary human B7-H6 is provided below:

B7-H6
(SEQ ID NO: 20)
MTWRAAASTCAALLILLWALTTEGDLKVEMMAGGTQITPLNDNVTIFCNI
FYSQPLNITSMGITWFWKSLTFDKEVKVFEFFGDHQEAFRPGAIVSPWRL
KSGDASLRLPGIQLEEAGEYRCEVVVTPLKAQGTVQLEVVASPASRLLLD
QVGMKENEDKYMCESSGFYPEAINITWEKQTQKFPHPIEISEDVITGPTI
KNMDGTFNVTSCLKLNSSQEDPGTVYQCVVRHASLHTPLRSNFTLTAARH
SLSETEKTDNFSIHWWPISFIGVGLVLLIVLIPWKKICNKSSSAYTPLKC
ILKHWNSFDTQTLKKEHLIFFCTRAWPSYQLQDGEAWPPEGSVNINTIQQ
LDVFCRQEGKWSEVPYVQAFFALRDNPDLCQCCRIDPALLTVTSGKSIDD
NSTKSEKQTPREHSDAVPDAPILPVSPIWEPPPATTSTTPVLSSQPPTLL
LPLQ

B7-H7 (HHLA2; HERV-H LTR-Associating 2) is a protein ligand found on the surface of monocytes. B7-H7 is thought to regulate cell-mediated immunity through binding a receptor on T lymphocytes and inhibiting proliferation in the same. The amino acid sequence of an exemplary human B7-H7 is provided below:

B7-H7
(SEQ ID NO: 21)
MKAQTALSFELILITSLSGSQGIFPLAFFIYVPMNEQIVIGRLDEDIILP
SSFERGSEVVIHWKYQDSYKVHSYYKGSDHLESQDPRYANRTSLFYNEIQ
NGNASLFFRRVSLLDEGIYTCYVGTAIQVITNKVVLKVGVELTPVMKYEK
RNTNSFLICSVLSVYPRPIITWKMDNTPISENNMEETGSLDSFSINSPLN
ITGSNSSYECTIENSLLKQTWTGRWTMKDGLHKMQSEHVSLSCQPVNDYF
SPNQDFKVTWSRMKSGTESVLAYYLSSSQNTIINESRFSWNKELINQSDF
SMNLMDLNLSDSGEYLCNISSDEYTLLTIHTVHVEPSQETASHNKGLWIL
VPSAILAAFLLIWSVKCCRAQLEARRSRHPADGAQQERCCVPPGERCPSA
PDNGEENVPLSGKV

4-1BB (CD137; TNFRSF9) is a tumor necrosis factor (TNF) superfamily member that is expressed by activated T cells. Crosslinking of 4-1BB enhances T cell proliferation, IL-2 secretion, survival, and cytolytic activity. The amino acid sequence of an exemplary human 4-1BB is provided below:

4-1BB
(SEQ ID NO: 22)
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

4-1BBL (TNFSF9; 4-1BB ligand) is a Type 2 transmembrane glycoprotein receptor belonging to the TNF superfamily 4-1BBL is expressed on activated T Lymphocytes and binds to 4-1BB. The amino acid sequence of certain exemplary human 4-1BBL polypeptides (including native and variants) are provided below:

4-1BBL
(SEQ ID NO: 23)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE
4-1BBL-CD
(lacking cytoplasmic domain; SEQ ID NO: 24)
MRVLPWALVAGLLLLLLLAAACAVFLACPWAVSGARASPGSAASPRLREG
PELSPDDPAGLLDLRQGMFAQLVAQNVLLIDGPLSWYSDPGLAGVSLTGG
LSYKEDTKELVVAKAGVYYVFFQLELRRVVAGEGSGSVSLALHLQPLRSA
AGAAALALTVDLPPASSEARNSAFGFQGRLLHLSAGQRLGVHLHTEARAR
HAWQLTQGATVLGLFRVTPEIPAGLPSPRSE
4-1BBL Q89A
(SEQ ID NO: 25)
MEYASDASLDPEAPWPPAPRARACRVLPWALVAGLLLLLLLAAACAVFLA
CPWAVSGARASPGSAASPRLREGPELSPDDPAGLLDLRAGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE
4-1BBL Q89A/CD (lacking cytoplasmic domain)
(SEQ ID NO: 26)
MRVLPWALVAGLLLLLLLAAACAVFLACPWAVSGARASPGSAASPRLREG
PELSPDDPAGLLDLRAGMFAQLVAQNVLLIDGPLSWYSDPGLAGVSLTGG
LSYKEDTKELVVAKAGVYYVFFQLELRRVVAGEGSGSVSLALHLQPLRSA
AGAAALALTVDLPPASSEARNSAFGFQGRLLHLSAGQRLGVHLHTEARAR
HAWQLTQGATVLGLFRVTPEIPAGLPSPRSE
4-1BBL L115A
(SEQ ID NO: 27)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGAAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE
4-1BBL L115A/CD
(SEQ ID NO: 28)
MRVLPWALVAGLLLLLLLAAACAVFLACPWAVSGARASPGSAASPRLREG
PELSPDDPAGLLDLRQGMFAQLVAQNVLLIDGPLSWYSDPGAAGVSLTGG
LSYKEDTKELVVAKAGVYYVFFQLELRRVVAGEGSGSVSLALHLQPLRSA
AGAAALALTVDLPPASSEARNSAFGFQGRLLHLSAGQRLGVHLHTEARAR
HAWQLTQGATVLGLFRVTPEIPAGLPSPRSE
4-1BBL K127A
(SEQ ID NO: 29)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYAEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE
4-1BBL Q227A
(SEQ ID NO: 30)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWALTQGATVLGLFRVTPEIPAGLPS
PRSE

BAFF (B-cell activating factor; TNFSF13B) is a member of the TNF ligand family and serves as a ligand for receptors TNFRSF13B/TACI, TNFRSF17/BCMA, and TNFRSF13C/BAFF-R. BAFF is a potent B cell activator and plays an important role in B cell proliferation and differentiation. The amino acid sequence of an exemplary human BAFF is provided below:

BAFF
(SEQ ID NO: 31)
MDDSTEREQSRLTSCLKKREEMKLKECVSILPRKESPSVRSSKDGKLLAA
TLLLALLSCCLTVVSFYQVAALQGDLASLRAELQGHHAEKLPAGAGAPKA
GLEEAPAVTAGLKIFEPPAPGEGNSSQNSRNKRAVQGPEETVTQDCLQLI
ADSETPTIQKGSYTFVPWLLSFKRGSALEEKENKILVKETGYFFIYGQVL
YTDKTYAMGHLIQRKKVHVFGDELSLVTLFRCIQNMPETLPNNSCYSAGI
AKLEEGDELQLAIPRENAQISLDGDVTFFGALKLL

BAFFR (B-cell activating factor receptor; TNFRSF13C) is a membrane protein of the TNF receptor superfamily and acts as a receptor for BAFF. BAFFR enhances B cell survival and is a regulator of the peripheral B-cell population. The amino acid sequence of an exemplary human BAFFR is provided below:

BAFFR
(SEQ ID NO: 32)
MRRGPRSLRGRDAPAPTPCVPAECFDLLVRHCVACGLLRTPRPKPAGASS
PAPRTALQPQESVGAGAGEAALPLPGLLFGAPALLGLALVLALVLVGLVS
WRRRQRRLRGASSAEAPDGDKDAPEPLDKVIILSPGISDATAPAWPPPGE
DPGTTPPGHSVPVPATELGSTELVTTKTAGPEQQ

CD27 (TNFRSF7) is a member of the TNF receptor superfamily and is required for generation and long-term maintenance of T cell immunity. CD27 binds to CD70 and also plays a role in regulation of B-cell activation and immunoglobulin synthesis. The amino acid sequence of an exemplary human CD27 is provided below:

CD27
(SEQ ID NO: 33)
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRILVIFSGME
LVETLAGALFLHQRRKYRSNKGESPVEPAEPCHYSCPREEEGSTIPIQED
YRKPEPACSP

CD70 (CD27LG; TNFSF7) is a protein expressed on highly activated lymphocytes. CD70 acts as a ligand for CD27. The amino acid sequence of an exemplary human CD70 is provided below:

CD70
(SEQ ID NO: 34)
MPEEGSGCSVRRRPYGCVLRAALVPLVAGLVICLVVCIQRFAQAQQQLPL
ESLGWDVAELQLNHTGPQQDPRLYWQGGPALGRSFLHGPELDKGQLRIHR
DGIYMVHIQVTLAICSSTTASRHHPTTLAVGICSPASRSISLLRLSFHQG
CTIVSQRLTPLARGDTLCTNLTGTLLPSRNTDETFFGVQWVRP

CD30 (TNFRSF8) is a member of the TNF receptor superfamily that is expressed by activated T cells and B cells. CD30 is a cell membrane protein that has been shown to interact with CD30L, TRAF1, TRAF2, TRAF3, and TRAF5. The amino acid sequence of an exemplary human CD30 is provided below:

CD30
(SEQ ID NO: 35)
MRVLLAALGLLFLGALRAFPQDRPFEDTCHGNPSHYYDKAVRRCCYRCPM
GLFPTQQCPQRPTDCRKQCEPDYYLDEADRCTACVTCSRDDLVEKTPCAW
NSSRVCECRPGMFCSTSAVNSCARCFFHSVCPAGMIVKFPGTAQKNTVCE
PASPGVSPACASPENCKEPSSGTIPQAKPTPVSPATSSASTMPVRGGTRL
AQEAASKLTRAPDSPSSVGRPSSDPGLSPTQPCPEGSGDCRKQCEPDYYL
DEAGRCTACVSCSRDDLVEKTPCAWNSSRTCECRPGMICATSATNSCARC
VPYPICAAETVTKPQDMAEKDTTFEAPPLGTQPDCNPTPENGEAPASTSP
TQSLLVDSQASKTLPIPTSAPVALSSTGKPVLDAGPVLFWVILVLVVVVG
SSAFLLCHRRACRKRIRQKLHLCYPVQTSQPKLELVDSRPRRSSTQLRSG
ASVTEPVAEERGLMSQPLMETCHSVGAAYLESLPLQDASPAGGPSSPRDL
PEPRVSTEHTNNKIEKIYIMKADTVIVGTVKAELPEGRGLAGPAEPELEE
ELEADHTPHYPEQETEPPLGSCSDVMLSVEEEGKEDPLPTAASGK

CD30L (CD30LG; TNFSF8) is a member of the TNF receptor superfamily CD30L acts as a ligand of CD30, and is expressed on induced T cells and monocytes/macrophages. The amino acid sequence of an exemplary human CD30L is provided below:

CD30L
(SEQ ID NO: 36)
MDPGLQQALNGMAPPGDTAMHVPAGSVASHLGTTSRSYFYLTTATLALCL
VFTVATIMVLVVQRTDSIPNSPDNVPLKGGNCSEDLLCILKRAPFKKSWA
YLQVAKHLNKTKLSWNKDGILHGVRYQDGNLVIQFPGLYFIICQLQFLVQ
CPNNSVDLKLELLINKHIKKQALVTVCESGMQTKHVYQNLSQFLLDYLQV
NTTISVNVDTFQYIDTSTFPLENVLSIFLYSNSD

CD40 (TNFRSF5) is a cell surface receptor expressed on the surface of B cells, monocytes, dendritic cells, endothelial cells, and epithelial cells. CD40 has been demonstrated to have involvement in T cell-dependent immunoglobulin class switching, memory B cell development, and germinal center formation. The amino acid sequence of an exemplary human CD40 is provided below:

CD40
(SEQ ID NO: 37)
MVRLPLQCVLWGCLLTAVHPEPPTACREKQYLINSQCCSLCQPGQKLVSD
CTEFTETECLPCGESEFLDTWNRETHCHQHKYCDPNLGLRVQQKGTSETD
TICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPI
IFGILFAILLVLVFIKKVAKKPTNKAPHPKQEPQEINFPDDLPGSNTAAP
VQETLHGCQPVTQEDGKESRISVQERQ

CD40L (CD40LG; TRAP; TNFSF5) is a member of the TNF superfamily expressed on B lymphocytes, epithelial cells, and some carcinoma cells. CD40L is a transmembrane protein that is known to interact with CD40 in order to mediate B cell proliferation, adhesion, and differentiation. The amino acid sequence of an exemplary human CD40L is provided below:

CD40L
(SEQ ID NO: 38)
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGETSFGLLKL

DR3 (TNFR25; APO3; TRAMP; LARD; WSL-1,) is a TNF receptor superfamily member expressed in lymphocytes. DR3 is thought to be the receptor responsible for TL1A-induced T cell co-stimulation. The amino acid sequence of an exemplary human DR3 is provided below:

DR3
(SEQ ID NO: 39)
MEQRPRGCAAVAAALLLVLLGARAQGGTRSPRCDCAGDFHKKIGLFCCRG
CPAGHYLKAPCTEPCGNSTCLVCPQDTFLAWENHHNSECARCQACDEQAS
QVALENCSAVADTRCGCKPGWFVECQVSQCVSSSPFYCQPCLDCGALHRH
TRLLCSRRDTDCGTCLPGFYEHGDGCVSCPTSTLGSCPERCAAVCGWRQM
FWVQVLLAGLVVPLLLGATLTYTYRHCWPHKPLVTADEAGMEALTPPPAT
HLSPLDSAHTLLAPPDSSEKICTVQLVGNSWTPGYPETQEALCPQVTWSW
DQLPSRALGPAAAPTLSPESPAGSPAMMLQPGPQLYDVMDAVPARRWKEF
VRTLGLREAEIEAVEVEIGRFRDQQYEMLKRWRQQQPAGLGAVYAALERM
GLDGCVEDLRSRLQRGP

GITR (Glucocorticoid-induced TNFR-related protein; AITR; TNFRSF18) is a member of the TNF receptor superfamily and is expressed in several cells and tissues including T lymphocytes, NK cells and antigen-presenting cells. GITR interaction with its ligand (GITRL) induces a co-activating signal. The amino acid sequence of an exemplary human GITR is provided below:

GITR
(SEQ ID NO: 40)
MAQHGAMGAFRALCGLALLCALSLGQRPTGGPGCGPGRLLLGTGTDARCC
RVHTTRCCRDYPGEECCSEWDCMCVQPEFHCGDPCCTTCRHHPCPPGQGV
QSQGKFSFGFQCIDCASGTFSGGHEGHCKPWTDCTQFGFLTVFPGNKTHN
AVCVPGSPPAEPLGWLTVVLLAVAACVLLLTSAQLGLHIWQLRSQCMWPR
ETQLLLEVPPSTEDARSCQFPEEERGERSAEEKGRLGDLW

GITRL (TNFSF18) is a cytokine belonging to the TNF ligand family and acts as a receptor for GITR. GITR interaction with its ligand (GITRL) induces a co-activating signal and has been shown to modulate T lymphocyte survival in peripheral tissues. The amino acid sequence of an exemplary human GITRL is provided below:

GITRL
(SEQ ID NO: 41)
MTLHPSPITCEFLFSTALISPKMCLSHLENMPLSHSRTQGAQRSSWKLWL
FCSIVMLLFLCSFSWLIFIFLQLETAKEPCMAKFGPLPSKWQMASSEPPC
VNKVSDWKLEILQNGLYLIYGQVAPNANYNDVAPFEVRLYKNKDMIQTLI
NKSKIQNVGGIYELHVGDTIDLIFNSEHQVLKNNTYWGIILLANPQFIS

HVEM (Herpesvirus entry mediator; TNFRSF14; CD270) is a cell surface receptor and a member of the TNF receptor superfamily. HVEM provides a stimulatory signal to T cells following engagement with LIGHT (TNFSF14); or an inhibitory signal to T cells when it binds the B and T lymphocyte attenuator (BTLA), a ligand member of the Immunoglobulin (Ig) superfamily. The amino acid sequence of an exemplary human HVEM is provided below:

HVEM
(SEQ ID NO: 42)
MEPPGDWGPPPWRSTPKTDVLRLVLYLTFLGAPCYAPALPSCKEDEYPVG
SECCPKCSPGYRVKEACGELIGTVCEPCPPGIYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH

LIGHT (TNFSF14; CD258; HVEML) is a member of the TNF ligand family that functions as a co-stimulatory factor along with HVEM. LIGHT has been demonstrated to stimulate the proliferation of T cells and trigger apoptosis of various tumor cells. The amino acid sequence of an exemplary human LIGHT is provided below:

LIGHT
(SEQ ID NO: 43)
MEESVVRPSVFVVDGQTDIPFTRLGRSHRRQSCSVARVGLGLLLLLMGAG
LAVQGWFLLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEEVVVRVLDERLVRLRDGTRSYFGAFMV

TNF-alpha (TNFSF2) is a member of the TNF ligand superfamily known to be secreted by, for example, macrophages and activated CD4-positive T cells. TNF-alpha is known to induce certain co-stimulatory molecules such as B7h and TNFRII. The amino acid sequence of an exemplary human TNF-alpha is provided below:

TNF-alpha
(SEQ ID NO: 44)
MSTESMIRDVELAEEALPKKTGGPQGSRRCLELSLFSFLIVAGATTLFCL
LHEGVIGPQREEFPRDLSLISPLAQAVRSSSRTPSDKPVAHVVANPQAEG
QLQWLNRRANALLANGVELRDNQLVVPSEGLYLIYSQVLFKGQGCPSTHV
LLTHTISRIAVSYQTKVNLLSAIKSPCQRETPEGAEAKPWYEPIYLGGVF
QLEKGDRLSAEINRPDYLDFAESGQVYFGIIAL

TNF-beta (TNFSF1; Lymphotoxin alpha) is a member of the TNF superfamily involved in the regulation of cell survival, proliferation, differentiation, and apoptosis. The amino acid sequence of an exemplary human TNF-beta is provided below:

TNF-beta
(SEQ ID NO: 45)
MTPPERLFLPRVCGTTLHLLLLGLLLVLLPGAQGLPGVGLTPSAAQTARQ
HPKMHLAHSTLKPAAHLIGDPSKQNSLLWRANTDRAFLQDGFSLSNNSLL
VPTSGIYFVYSQVVFSGKAYSPKATSSPLYLAHEVQLFSSQYPFHVPLLS
SQKMVYPGLQEPWLHSMYHGAAFQLTQGDQLSTHTDGIPHLVLSPSTVFF
GAFAL

OX40 (TNFRSF4; CD134) is a member of the TNF receptor superfamily OX40 binds to OX40L and contributes to T cell expansion, survival, and cytokine production. The amino acid sequence of an exemplary human OX40 is provided below:

OX40
(SEQ ID NO: 46)
MCVGARRLGRGPCAALLLLGLGLSTVTGLHCVGDTYPSNDRCCHECRP
GNGMVSRCSRSQNTVCRPCGPGFYNDVVSSKPCKPCTWCNLRSGSERK
QLCTATQDTVCRCRAGTQPLDSYKPGVDCAPCPPGHFSPGDNQACKPW
TNCTLAGKHTLQPASNSSDAICEDRDPPATQPQETQGPPARPITVQPT
EAWPRTSQGPSTRPVEVPGGRAVAAILGLGLVLGLLGPLAILLALYLL
RRDQRLPPDAHKPPGGGSFRTPIQEEQADAHSTLAKI

OX40L (TNFSF4; CD252) is a member of the TNF ligand superfamily and is expressed, for example, on activated CD4 and CD8 T cells as well as a number of other lymphoid and non-lymphoid cells. OX40L interacts with OX40 in order to regulate, for example, T cell expansion, survival, and cytokine production. The amino acid sequence of an exemplary human OX40L is provided below:

OX40L
(SEQ ID NO: 47)
MERVQPLEENVGNAARPRFERNKLLLVASVIQGLGLLLCFTYICLHFS
ALQVSHRYPRIQSIKVQFTEYKKEKGFILTSQKEDEIMKVQNNSVIIN
CDGFYLISLKGYFSQEVNISLHYQKDEEPLFQLKKVRSVNSLMVASLT
YKDKVYLNVTTDNTSLDDFHVNGGELILIHQNPGEFCVL

RELT (TNFRSF19L) is a member of the TNF receptor superfamily. RELT is a type I transmembrane glycoprotein and is thought to be capable of co-stimulating T cell proliferation in the presence of CD3 signaling. The amino acid sequence of an exemplary human RELT is provided below:

RELT
(SEQ ID NO: 48)
MKPSLLCRPLSCFLMLLPWPLATLTSTTLWQCPPGEEPDLDPGQGTLC
RPCPPGTFSAAWGSSPCQPHARCSLWRRLEAQVGMATRDTLCGDCWPG
WFGPWGVPRVPCQPCSWAPLGTHGCDEWGRRARRGVEVAAGASSGGET
RQPGNGTRAGGPEETAAQYAVIAIVPVFCLMGLLGILVCNLLKRKGYH
CTAHKEVGPGPGGGGSGINPAYRTEDANEDTIGVLVRLITEKKENAAA
LEELLKEYHSKQLVQTSHRPVSKLPPAPPNVPHICPHRHHLHTVQGLA
SLSGPCCSRCSQKKWPEVLLSPEAVAATTPVPSLLPNPTRVPKAGAKA
GRQGEITILSVGRFRVARIPEQRTSSMVSEVKTITEAGPSWGDLPDSP
QPGLPPEQQALLGSGGSRTKWLKPPAENKAEENRYVVRLSESNLVI

TACI (Transmembrane activator and CAML interactor; TNFRSF13B; CD267) is a TNF receptor superfamily member that is found, for example, on the surface of B cells. TACI is known to interact with ligands BAFF and APRIL. The amino acid sequence of an exemplary human TACI is provided below:

TACI
(SEQ ID NO: 49)
MSGLGRSRRGGRSRVDQEERFPQGLWTGVAMRSCPEEQYWDPLLGTCM
SCKTICNHQSQRTCAAFCRSLSCRKEQGKFYDHLLRDCISCASICGQH
PKQCAYFCENKLRSPVNLPPELRRQRSGEVENNSDNSGRYQGLEHRGS
EASPALPGLKLSADQVALVYSTLGLCLCAVLCCFLVAVACFLKKRGDP
CSCQPRSRPRQSPAKSSQDHAMEAGSPVSTSPEPVETCSFCFPECRAP
TQESAVTPGTPDPTCAGRWGCHTRTTVLQPCPHIPDSGLGIVCVPAQE
GGPGA

TL1A (TNFSF15) is a member of the TNF ligand superfamily that is known to bind to DR3. TL1A can act to enhance T cell proliferation and cytokine production of T cells. The amino acid sequence of an exemplary human TL1A is provided below:

TL1A
(SEQ ID NO: 50)
MAEDLGLSFGETASVEMLPEHGSCRPKARSSSARWALTCCLVLLPFLA
GLTTYLLVSQLRAQGEACVQFQALKGQEFAPSHQQVYAPLRADGDKPR
AHLTVVRQTPTQHFKNQFPALHWEHELGLAFTKNRMNYTNKFLLIPES
GDYFIYSQVTFRGMTSECSEIRQAGRPNKPDSITVVITKVTDSYPEPT
QLLMGTKSVCEVGSNWFQPIYLGAMFSLQEGDKLMVNVSDISLVDYTK
EDKTFFGAFLL

TNFRII (TNFRSF1B) is a TNF receptor superfamily member that binds to TNF-alpha. TNFRII has been shown to act as a co-stimulatory receptor for T cells and as a critical factor for the development of regulatory T cells (Treg) and myeloid suppressor cells. The amino acid sequence of an exemplary human TNFRII is provided below:

TNFRII
(SEQ ID NO: 51)
MAPVAVWAALAVGLELWAAAHALPAQVAFTPYAPEPGSTCRLREYYDQ
TAQMCCSKCSPGQHAKVFCTKTSDTVCDSCEDSTYTQLWNWVPECLSC
GSRCSSDQVETQACTREQNRICTCRPGWYCALSKQEGCRLCAPLRKCR
PGFGVARPGTETSDVVCKPCAPGTFSNTTSSTDICRPHQICNVVAIPG
NASMDAVCTSTSPTRSMAPGAVHLPQPVSTRSQHTQPTPEPSTAPSTS
FLLPMGPSPPAEGSTGDFALPVGLIVGVTALGLLIIGVVNCVIMTQVK
KKPLCLQREAKVPHLPADKARGTQGPEQQHLLITAPSSSSSSLESSAS
ALDRRAPTRNQPQAPGVEASGAGEARASTGSSDSSPGGHGTQVNVTCI
VNVCSSSDHSSQCSSQASSTMGDTDSSPSESPKDEQVPFSKEECAFRS
QLETPETLLGSTEEKPLPLGVPDAGMKPS

BCMA is a cell surface receptor of the TNF receptor superfamily, and binds to the tumor necrosis factor superfamily, member 13b (TNFSF13B), leading to NF-kappaB and MAPK8/JNK activation. It is preferentially expressed on mature B lymphocytes and plays a pivotal role in B cell development, function, and regulation. The amino acid sequence of an exemplary human BCMA is provided below:

BCMA
(SEQ ID NO: 52)
MLQMAGQCSQNEYFDSLLHACIPCQLRCSSNTPPLTCQRYCNASVTNS
VKGTNAILWTCLGLSLIISLAVFVLMFLLRKINSEPLKDEFKNTGSGL
LGMANIDLEKSRTGDEIILPRGLEYTVEECTCEDCIKSKPKVDSDHCF
PLPAMEEGATILVTTKTNDYCKSLPAALSATEIEKSISAR

EDA2R is a type III transmembrane protein of the TNFR (tumor necrosis factor receptor) superfamily and contains 3 cysteine-rich repeats and one transmembrane domain. It binds to the EDA-A2 isoform of the ectodysplasin, playing an important role in maintaining hair and teeth. The amino acid sequence of an exemplary human EDA2R is provided below:

EDA2R
(SEQ ID NO: 53)
MDCQENEYWDQWGRCVTCQRCGPGQELSKDCGYGEGGDAYCTACPPRR
YKSSWGHHRCQSCITCAVINRVQKVNCTATSNAVCGDCLPRFYRKTRI
GGLQDQECIPCTKQTPTSEVQCAFQLSLVEADTPTVPPQEATLVALVS
SLLVVFTLAFLGLFFLYCKQFFNRHCQRGGLLQFEADKTAKEESLFPV
PPSKETSAESQVSENIFQTQPLNPILEDDCSSTSGFPTQESFTMASCT
SESHSHWVHSPIECTELDLQKFSSSASYTGAETLGGNTVESTGDRLEL
NVPFEVPSP

TROY or TNFR (tumor necrosis factor receptor) superfamily member 19 is a type 1 cell surface receptor that is highly expressed in the embryonic and adult CNS and developing hair follicles. It activates the JNK signaling pathway when overexpressed in cells, interacts with TRAF family members, and can induce apoptosis by a caspace-independent mechanism. The amino acid sequence of an exemplary human TROY is provided below:

TROY
(SEQ ID NO: 54)
MALKVLLEQEKTFFTLLVLLGYLSCKVTCESGDCRQQEFRDRSGNCVP
CNQCGPGMELSKECGFGYGEDAQCVTCRLHRFKEDWGFQKCKPCLDCA
VVNRFQKANCSATSDAICGDCLPGFYRKTKLVGFQDMECVPCGDPPPP
YEPHCASKVNLVKIASTASSPRDTALAAVICSALATVLLALLILCVIY
CKRQFMEKKPSWSLRSQDIQYNGSELSCFDRPQLHEYAHRACCQCRRD
SVQTCGPVRLLPSMCCEEACSPNPATLGCGVHSAASLQARNAGPAGEM
VPTFFGSLTQSICGEFSDAWPLMQNPMGGDNISFCDSYPELTGEDIHS
LNPELESSTSLDSNSSQDLVGGAVPVQSHSENFTAATDLSRYNNTLVE
SASTQDALTMRSQLDQESGAVIHPATQTSLQVRQRLGSL

LTBR or tumor necrosis factor receptor superfamily member 3 (TNFRSF3) is a cell surface receptor that binds to the lymphotoxin membrane form (a complex of lymphotoxin-alpha and lymphtoxin-beta). It plays a role in apoptosis, lipid metabolism, and the development and organization of lymphoid tissue and transformed cells. The amino acid sequence of an exemplary human LTBR is provided below:

LTBR
(SEQ ID NO: 55)
MLLPWATSAPGLAWGPLVLGLFGLLAASQPQAVPPYASENQTCRDQEK
EYYEPQHRICCSRCPPGTYVSAKCSRIRDTVCATCAENSYNEHWNYLT
ICQLCRPCDPVMGLEEIAPCTSKRKTQCRCQPGMFCAAWALECTHCEL
LSDCPPGTEAELKDEVGKGNNHCVPCKAGHFQNTSSPSARCQPHTRCE
NQGLVEAAPGTAQSDTTCKNPLEPLPPEMSGTMLMLAVLLPLAFFLLL
ATVFSCIWKSHPSLCRKLGSLLKRRPQGEGPNPVAGSWEPPKAHPYFP
DLVQPLLPISGDVSPVSTGLPAAPVLEAGVPQQQSPLDLTREPQLEPG
EQSQVAHGTNGIHVTGGSMTITGNIYIYNGPVLGGPPGPGDLPATPEP
PYPIPEEGDPGPPGLSTPHQEDGKAWHLAETEHCGATPSNRGPRNQFI
THD

EDAR (Ectodysplasin A receptor) is a cell surface receptor for ectodysplasin A and plays a pivotal role in embryonic development, as well as the development of hair, teeth, and other ectodermal derivatives. It can activate the nuclear factor-kappaB, JNK, and caspase-independent cell death pathways. The amino acid sequence of an exemplary human EDAR is provided below:

EDAR
(SEQ ID NO: 56)
MAHVGDCTQTPWLPVLVVSLMCSARAEYSNCGENEYYNQTTGLCQECP
PCGPGEEPYLSCGYGTKDEDYGCVPCPAEKFSKGGYQICRRHKDCEGF
FRATVLTPGDMENDAECGPCLPGYYMLENRPRNIYGMVCYSCLLAPPN
TKECVGATSGASANFPGTSGSSTLSPFQHAHKELSGQGHLATALIIAM
STIFIMAIAIVLIIMFYILKTKPSAPACCTSHPGKSVEAQVSKDEEKK
EAPDNVVMFSEKDEFEKLTATPAKPTKSENDASSENEQLLSRSVDSDE
EPAPDKQGSPELCLLSLVHLAREKSATSNKSAGIQSRRKKILDVYANV
CGVVEGLSPTELPFDCLEKTSRMLSSTYNSEKAVVKTWRHLAESFGLK
RDEIGGMTDGMQLFDRISTAGYSIPELLTKLVQIERLDAVESLCADIL
EWAGVVPPASQPHAAS

NGFR (Nerve Growth Factor Receptor) is a low affinity cell surface receptor for the neurotrophins, which are protein growth factors that stimulate neuronal cell survival and differentiation. NGFR also binds pro-neurotrophins and functions as a co-receptor with other receptor partners, including SORT1 (Sortilin), LINGO1, and RTN4R. It has broad expression in the spleen, adrenal, and brain, among other tissues. The amino acid sequence of an exemplary human NGFR is provided below:

NGFR
(SEQ ID NO: 57)
MGAGATGRAMDGPRLLLLLLLGVSLGGAKEACPTGLYTHSGECCKACN
LGEGVAQPCGANQTVCEPCLDSVTFSDVVSATEPCKPCTECVGLQSMS
APCVEADDAVCRCAYGYYQDETTGRCEACRVCEAGSGLVFSCQDKQNT
VCEECPDGTYSDEANHVDPCLPCTVCEDTERQLRECTRWADAECEEIP
GRWITRSTPPEGSDSTAPSTQEPEAPPEQDLIASTVAGVVTTVMGSSQ
PVVTRGTTDNLIPVYCSILAAVVVGLVAYIAFKRWNSCKQNKQGANSR
PVNQTPPPEGEKLHSDSGISVDSQSLHDQQPHTQTASGQALKGDGGLY
SSLPPAKREEVEKLLNGSAGDTWRHLAGELGYQPEHIDSFTHEACPVR
ALLASWATQDSATLDALLAALRRIQRADLVESLCSESTATSPV

OPG (osteoprotegerin) is a cytokine receptor of tumor necrosis factor (TNF) receptor superfamily encoded by the TNFRSF11B gene that binds to TNF-related apoptosis-inducing ligand (TRAIL) and inhibits TRAIL-induced apoptosis of specific cells, including tumor cells. It functions as a negative regulator of bone resorption and plays an important role in osteoclast development, tumor growth and metastasis, heart disease, immune system development and signaling, mental health, diabetes, and the prevention of pre-eclampsia and osteoporosis during pregnancy. The amino acid sequence of an exemplary human OPG is provided below:

OPG
(SEQ ID NO: 58)
MNNLLCCALVFLDISIKWTTQETFPPKYLHYDEETSHQLLCDKCPPGT
YLKQHCTAKWKTVCAPCPDHYYTDSWHTSDECLYCSPVCKELQYVKQE
CNRTHNRVCECKEGRYLEIEFCLKHRSCPPGFGVVQAGTPERNTVCKR
CPDGFFSNETSSKAPCRKHTNCSVFGLLLTQKGNATHDNICSGNSEST
QKCGIDVTLCEEAFFRFAVPTKFTPNWLSVLVDNLPGTKVNAESVERI
KRQHSSQEQTFQLLKLWKHQNKDQDIVKKIIQDIDLCENSVQRHIGHA
NLTFEQLRSLMESLPGKKVGAEDIEKTIKACKPSDQILKLLSLWRIKN
GDQDTLKGLMHALKHSKTYHFPKTVTQSLKKTIRFLHSFTMYKLYQKL
FLEMIGNQVQSVKISCL

RANK (Receptor activator of nuclear factor κ B) is the receptor for RANK-Ligand (RANKL) and part of the RANK/RANKL/OPG signaling pathway that regulates osteoclast differentiation and activation. It is an important regulator of the interaction between T cells and dendritic cells and it plays an important role in bone remodeling and repair, immune cell function, lymph node development, thermal regulation, and mammary gland development. The amino acid sequence of an exemplary human RANK is provided below:

RANK
(SEQ ID NO: 59)
MAPRARRRRPLFALLLLCALLARLQVALQIAPPCTSEKHYEHLGRCCN
KCEPGKYMSSKCTTTSDSVCLPCGPDEYLDSWNEEDKCLLHKVCDTGK
ALVAVVAGNSTTPRRCACTAGYHWSQDCECCRRNTECAPGLGAQHPLQ
LNKDTVCKPCLAGYFSDAFSSTDKCRPWTNCTFLGKRVEHHGTEKSDA
VCSSSLPARKPPNEPHVYLPGLIILLLFASVALVAAIIFGVCYRKKGK
ALTANLWHWINEACGRLSGDKESSGDSCVSTHTANFGQQGACEGVLLL
TLEEKTFPEDMCYPDQGGVCQGTCVGGGPYAQGEDARMLSLVSKTEIE
EDSFRQMPTEDEYMDRPSQPTDQLLFLTEPGSKSTPPFSEPLEVGEND
SLSQCFTGTQSTVGSESCNCTEPLCRTDWTPMSSENYLQKEVDSGHCP
HWAASPSPNWADVCTGCRNPPGEDCEPLVGSPKRGPLPQCAYGMGLPP
EEEASRTEARDQPEDGADGRLPSSARAGAGSGSSPGGQSPASGNVTGN
SNSTFISSGQVMNFKGDIIVVYVSQTSQEGAAAAAEPMGRPVQEETLA
RRDSFAGNGPRFPDPCGGPEGLREPEKASRPVQEQGGAKA

DCR3 (Decoy receptor 3) is a soluble protein of the tumor necrosis factor receptor superfamily which plays a regulatory role in suppressing FasL- and LIGHT-mediated cell death and is a decoy receptor that competes with death receptors for ligand binding. It is overexpressed in gastrointestinal tract tumors. The amino acid sequence of an exemplary human DCR3 is provided below:

DCR3
(SEQ ID NO: 60)
MRALEGPGLSLLCLVLALPALLPVPAVRGVAETPTYPWRDAETGERLV
CAQCPPGTFVQRPCRRDSPTTCGPCPPRHYTQFWNYLERCRYCNVLCG
EREEEARACHATHNRACRCRTGFFAHAGFCLEHASCPPGAGVIAPGTP
SQNTQCQPCPPGTFSASSSSSEQCQPHRNCTALGLALNVPGSSSHDTL
CTSCTGFPLSTRVPGAEECERAVIDFVAFQDISIKRLQRLLQALEAPE
GWGPTPRAGRAALQLKLRRRLTELLGAQDGALLVRLLQALRVARMPGL
ERSVRERFLPVH

TNFR1 (Tumor necrosis factor receptor 1) is a ubiquitous membrane receptor that binds tumor necrosis factor-alpha (TNFα), which can activate the transcription factor NF-κB, mediate apoptosis, and function as a regulator of inflammation. The amino acid sequence of an exemplary human TNFR1 is provided below:

TNFR1
(SEQ ID NO: 61)
MGLSTVPDLLLPLVLLELLVGIYPSGVIGLVPHLGDREKRDSVCPQGKYI
HPQNNSICCTKCHKGTYLYNDCPGPGQDTDCRECESGSFTASENHLRHCL
SCSKCRKEMGQVEISSCTVDRDTVCGCRKNQYRHYWSENLFQCFNCSLCL
NGTVHLSCQEKQNTVCTCHAGEFLRENECVSCSNCKKSLECTKLCLPQIE
NVKGTEDSGTTVLLPLVIFFGLCLLSLLFIGLMYRYQRWKSKLYSIVCGK
STPEKEGELEGTTTKPLAPNPSFSPTPGETPTLGFSPVPSSTFTSSSTYT
PGDCPNFAAPRREVAPPYQGADPILATALASDPIPNPLQKWEDSAHKPQS
LDTDDPATLYAVVENVPPLRWKEFVRRLGLSDHEIDRLELQNGRCLREAQ
YSMLATWRRRTPRREATLELLGRVLRDMDLLGCLEDIEEALCGPAALPPA
PSLLR

FN14 (Fibroblast growth factor-inducible 14) is induced in a variety of cell types in situations of tissue injury and is activated by TNF-like weak inducer of apoptosis (TWEAK), a member of the TNF ligand family that controls many cellular activities including proliferation, migration, differentiation, apoptosis, angiogenesis and inflammation. NFAT1 regulates the expression of FN14 and its ligand TWEAK with lipocalin 2 to increase breast cancer cell invasion. The amino acid sequence of an exemplary human FN14 is provided below:

FN14
(SEQ ID NO: 62)
MARGSLRRLLRLLVLGLWLALLRSVAGEQAPGTAPCSRGSSWSADLDKCM
DCASCRARPHSDFCLGCAAAPPAPFRLLWPILGGALSLTFVLGLLSGFLV
WRRCRRREKFTTPIEETGGEGCPAVALIQ

APRIL (A proliferation-inducing ligand) is a ligand for TNFRSF17/BCMA, a member of the TNF receptor family Both APRIL and its receptor are important for B cell development. It is expressed at low levels in lymphoid tissue and is over-expressed by a number of tumors. The amino acid sequence of an exemplary human APRIL is provided below:

APRIL
(SEQ ID NO: 63)
MPASSPFLLAPKGPPGNMGGPVREPALSVALWLSWGAALGAVACAMALLT
QQTELQSLRREVSRLQGTGGPSQNGEGYPWQSLPEQSSDALEAWENGERS
RKRRAVLTQKQKKQHSVLHLVPINATSKDDSDVTEVMWQPALRRGRGLQA
QGYGVRIQDAGVYLLYSQVLFQDVTFTMGQVVSREGQGRQETLFRCIRSM
PSHPDRAYNSCYSAGVFHLHQGDILSVIIPRARAKLNLSPHGTFLGFVKL

EDA-A2 is a type II transmembrane protein that is a member of the TNF Superfamily (TNFSF) and acts as a homotrimer that may be involved in cell-cell signaling during the development of ectodermal organs. Defects in this gene are a cause of ectodermal dysplasia, anhidrotic, which is also known as X-linked hypohidrotic ectodermal dysplasia. The amino acid sequence of an exemplary human EDA-A2 is provided below:

EDA-A2
(SEQ ID NO: 64)
MGYPEVERRELLPAAAPRERGSQGCGCGGAPARAGEGNSCLLFLGFFGLS
LALHLLTLCCYLELRSELRRERGAESRLGGSGTPGTSGTLSSLGGLDPDS
PITSHLGQPSPKQQPLEPGEAALHSDSQDGHQMALLNFFFPDEKPYSEEE
SRRVRRNKRSKSNEGADGPVKNKKKGKKAGPPGPNGPPGPPGPPGPQGPP
GIPGIPGIPGTTVMGPPGPPGPPGPQGPPGLQGPSGAADKAGTRENQPAV
VHLQGQGSAIQVKNDLSGGVLNDWSRITMNPKVFKLHPRSGELEVLVDGT
YFIYSQVYYINFTDFASYEVVVDEKPFLQCTRSIETGKTNYNTCYTAGVC
LLKARQKIAVKMVHADISINMSKHTTFFGAIRLGEAPAS

TWEAK (TNF-related weak inducer of apoptosis) is a cytokine that belongs to the tumor necrosis factor (TNF) ligand family and a ligand for the FN14/TWEAKR receptor. It has overlapping signaling functions with TNF, but displays a much wider tissue distribution. It plays an important role in apoptosis, proliferation and migration of endothelial cells, and angiogenesis. The amino acid sequence of an exemplary human TWEAK is provided below:

TWEAK
(SEQ ID NO: 65)
MAARRSQRRRGRRGEPGTALLVPLALGLGLALACLGLLLAVVSLGSRASL
SAQEPAQEELVAEEDQDPSELNPQTEESQDPAPFLNRLVRPRRSAPKGRK
TRARRAIAAHYEVHPRPGQDGAQAGVDGTVSGWEEARINSSSPLRYNRQI
GEFIVTRAGLYYLYCQVHFDEGKAVYLKLDLLVDGVLALRCLEEFSATAA
SSLGPQLRLCQVSGLLALRPGSSLRIRTLPWAHLKAAPFLTYFGLFQVH

LTA (Lymphotoxin-alpha) is a cytokine produced by lymphocytes, and exists in both a membrane bound and soluble state. It forms heterotrimers with lymphotoxin-beta which anchor lymphotoxin-alpha to the cell surface, is involved in the formation of secondary lymphoid organs, and mediates a large variety of inflammatory, immunostimulatory, and antiviral responses. The amino acid sequence of an exemplary human LTA is provided below:

LTB
(SEQ ID NO: 66)
MGALGLEGRGGRLQGRGSLLLAVAGATSLVTLLLAVPITVLAVLALVPQD
QGGLVTETADPGAQAQQGLGFQKLPEEEPETDLSPGLPAAHLIGAPLKGQ
GLGWETTKEQAFLTSGTQFSDAEGLALPQDGLYYLYCLVGYRGRAPPGGG
DPQGRSVTLRSSLYRAGGAYGPGTPELLLEGAETVTPVLDPARRQGYGPL
WYTSVGFGGLVQLRRGERVYVNISHPDMVDFARGKTFFGAVMVG

NGF (Nerve growth factor) is a neurotrophic factor and neuropeptide primarily involved in the regulation of growth, maintenance, proliferation, and survival of certain target neurons. More specifically, NGF is critical for the survival of the sympathetic and sensory neurons. The amino acid sequence of an exemplary human NGF is provided below:

NGF
(SEQ ID NO: 67)
MSMLFYTLITAFLIGIQAEPHSESNVPAGHTIPQAHWTKLQHSLDTALRR
ARSAPAAAIAARVAGQTRNITVDPRLFKKRRLRSPRVLFSTQPPREAADT
QDLDFEVGGAAPFNRTHRSKRSSSHPIFHRGEFSVCDSVSVWVGDKTTAT
DIKGKEVMVLGEVNINNSVFKQYFFETKCRDPNPVDSGCRGIDSKHWNSY
CTTTHTFVKALTMDGKQAAWRFIRIDTACVCVLSRKAVRRA

EDA-A1 is a type II transmembrane protein belonging to the TNF superfamily that acts as a homotrimer and may be involved in cell-cell signaling during the development of ectodermal organs. The attachment of EDA-A1 to the ectodysplasin A receptor triggers a series of chemical signals that affect cell activities such as division, growth, and maturation. The amino acid sequence of an exemplary human EDA-A1 is provided below:

EDA-A1
(SEQ ID NO: 68)
MGYPEVERRELLPAAAPRERGSQGCGCGGAPARAGEGNSCLLFLGFFGLS
LALHLLTLCCYLELRSELRRERGAESRLGGSGTPGTSGTLSSLGGLDPDS
PITSHLGQPSPKQQPLEPGEAALHSDSQDGHQMALLNFFFPDEKPYSEEE
SRRVRRNKRSKSNEGADGPVKNKKKGKKAGPPGPNGPPGPPGPPGPQGPP
GIPGIPGIPGTTVMGPPGPPGPPGPQGPPGLQGPSGAADKAGTRENQPAV
VHLQGQGSAIQVKNDLSGGVLNDWSRITMNPKVFKLHPRSGELEVLVDGT
YFIYSQVEVYYINFTDFASYEVVVDEKPFLQCTRSIETGKTNYNTCYTAG
VCLLKARQKIAVKMVHADISINMSKHTTFFGAIRLGEAPAS

APP (amyloid precursor protein) is an integral membrane protein expressed in many tissues and concentrated in the synapses of neurons. It is expressed in many tissues, including the brain and spinal cord, and metabolized in a rapid and highly complex fashion by a series of sequential proteases, including the intramembranous γ-secretase complex, which also process other key regulatory molecules. The amino acid sequence of an exemplary human APP is provided below:

APP
(SEQ ID NO: 69)
MLPGLALLLLAAWTARALEVPTDGNAGLLAEPQIAMFCGRLNMHMNVQNG
KWDSDPSGTKTCIDTKEGILQYCQEVYPELQITNVVEANQPVTIQNWCKR
GRKQCKTHPHEVIPYRCLVGEFVSDALLVPDKCKFLHQERMDVCETHLHW
HTVAKETCSEKSTNLHDYGMLLPCGIDKFRGVEFVCCPLAEESDNVDSAD
AEEDDSDVWWGGADTDYADGSEDKVVEVAEEEEVAEVEEEEADDDEDDED
GDEVEEEAEEPYEEATERTTSIATTTTTTTESVEEVVREVCSEQAETGPC
RAMISRWYFDVTEGKCAPFFYGGCGGNRNNFDTEEYCMAVCGSAMSQSLL
KTTQEPLARDPVKLPTTAASTPDAVDKYLETPGDENEHAHFQKAKERLEA
KHRERMSQVMREWEEAERQAKNLPKADKKAVIQHFQEKVESLEQEAANER
QQLVETHMARVEAMLNDRRRLALENYITALQAVPPRPRHVFNMLKKYVRA
EQKDRQHTLKHFEHVRMVDPKKAAQIRSQVMTHLRVIYERMNQSLSLLYN
VPAVAEEIQDEVDELLQKEQNYSDDVLANMISEPRISYGNDALMPSLTET
KTTVELLPVNGEFSLDDLQPWHSFGADSVPANTENEVEPVDARPAADRGL
TTRPGSGLTNIKTEEISEVKMDAEFRHDSGYEVHHQKLVFFAEDVGSNKG
AIIGLMVGGVVIATVIVITLVMLKKKQYTSIHHGVVEVDAAVTPEERHLS
KMQQNGYENPTYKFFEQMQN

TRAIL (TNF-related apoptosis-inducing ligand) is a cytokine that induces apoptosis. It binds to two death receptors DR4 (TRAIL-RI) and DR5 (TRAIL-RII), and two decoy receptors DcR1 and DcR2. TRAIL functions by binding to the death receptors, recruiting the FAS-associated death domain, and activating caspases 8 and 10, which results in apoptosis. The amino acid sequence of an exemplary human TRAIL is provided below:

TRAIL
(SEQ ID NO: 70)
MAMMEVQGGPSLGQICVLIVIFTVLLQSLCVAVTYVYFTNELKQMQDKYS
KSGIACELKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG

B7-H4, also known as V-set domain-containing T-cell activation inhibitor 1 (VTCN1) is a member of the B7 family. This protein is found to be expressed on the surface of antigen-presenting cells and to interact with ligands such as CD28 or MIM 186760 on T cells. The amino acid sequence of an exemplary human B7-H4 is provided below:

B7-H4
(SEQ ID NO: 71)
MASLGQILFWSIISIIIILAGAIALIIGFGISGRHSITVITVASAGNIGE
DGILSCTFEPDIKLSDIVIQWLKEGVLGLVHEFKEGKDELSEQDEMERGR
TAVFADQVIVGNASLRLKNVQLTDAGTYKCYIITSKGKGNANLEYKTGAF
SMPEVNVDYNASSETLRCEAPRWFPQPTVVWASQVDQGANFSEVSNTSFE
LNSENVTMKVVSVLYNVTINNTYSCMIENDIAKATGDIKVTESEIKRRSH
LQLLNSKASLCVSSFFAISWALLPLSPYLMLK

In specific examples, the co-stimulatory polypeptide for use in the present disclosures include CD30L, CD40, CD40L, CD27, CD70, GITRL, ICOS, ICOSL, LIGHT, OX40, OX40L, TL1A, BAFFR, 4-1BB, or 4-1BBL. In some instances, the co-stimulatory polypeptide for use in the present disclosure is not CD80 or CD86.

The co-stimulatory polypeptide may be a naturally-occurring polypeptide from a suitable species, for example, a mammalian co-stimulatory polypeptide such as those derived from human or a non-human primate. Such naturally-occurring polypeptides are known in the art and can be obtained, for example, using any of the above-noted amino acid sequences as a query to search a publicly available gene database, for example GenBank. The co-stimulatory polypeptide for use in the instant disclosure may share a sequence identity of at least 85% (e.g., 90%, 95%, 97%, 98%, 99%, or above) with any of the exemplary proteins noted above. In some embodiments, the member of the B7/CD28 superfamily, member of the tumor necrosis factor (TNF) superfamily, or ligand thereof may lack a cytoplasmic domain. In an exemplary embodiment, the 4-1BBL lacks a cytoplasmic domain. In some embodiments, the member of the TNF superfamily or ligand thereof is not 4-1BBL.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Alternatively, the co-stimulatory polypeptide may be a functional variant of a native counterpart. Such a functional variant may contain one or more mutations within the functional domain(s) (e.g., within the active site of an enzyme) of the native counterpart. Such a functional variant may contain one or more mutations outside the functional domain(s) of the native counterpart. Functional domains of a native co-stimulatory polypeptide may be known in the art or can be predicted based on its amino acid sequence. Mutations outside the functional domain(s) would not be expected to substantially affect the biological activity of the protein. In some instances, the functional variant may have the capacity to modulate (i.e., stimulate) co-stimulatory pathways relative to the native counterpart.

Alternatively or in addition, the functional variant may contain a conservative mutation(s) at one or more positions in the native counterpart (e.g., up to 20 positions, up to 15 positions, up to 10 positions, up to 5, 4, 3, 2, 1 position(s)). As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The co-stimulatory polypeptide described herein may not require chemical induced (e.g., rimiducid-induced) dimerization to regulate the activity of the immune cells expressing such. For example, the co-stimulatory polypeptide may be free of a F506 binding protein (FKBP) or a fragment thereof (e.g., theFKBPv36 domain), which allows for dimerization induced by rimiducid.

### II. Anti-GPC3 CAR Polypeptides

As used herein, a CAR polypeptide (a.k.a., a CAR construct) refers to a non-naturally occurring molecule that can be expressed on the surface of a host cell and comprises an extracellular antigen binding domain, a transmembrane domain, and a cytoplasmic signaling domain. The extracellular antigen binding domain may be any peptide or polypeptide that specifically binds to (i.e., is specific to) a target antigen, including naturally occurring antigens that are associated with a medical condition (e.g., a disease), or an antigenic moiety conjugated to a therapeutic agent that targets a disease-associated antigen.

In some embodiments, the CAR polypeptides described herein may further include at least one co-stimulatory signaling domain. The CAR polypeptides are configured such that, when expressed on a host cell, the extracellular antigen-binding domain is located extracellularly for binding to a target molecule and the cytoplasmic signaling domain. The optional co-stimulatory signaling domain may be located in the cytoplasm for triggering activation and/or effector signaling.

In some embodiments, a CAR polypeptide as described herein may comprise, from N-terminus to C-terminus, the extracellular antigen-binding domain, the transmembrane domain, and the cytoplasmic signaling domain. In some embodiments, a CAR polypeptide as described herein comprises, from N-terminus to C-terminus, the extracellular antigen-binding domain, the transmembrane domain, at least one co-stimulatory signaling domain, and the cytoplasmic signaling domain. In other embodiments, a CAR polypeptide as described herein comprises, from N-terminus to C-terminus, the extracellular antigen-binding domain, the transmembrane domain, the cytoplasmic signaling domains, and at least one co-stimulatory signaling domain.

As used herein, the phrase “a protein X transmembrane domain” (e.g., a CD8 transmembrane domain) refers to any portion of a given protein, i.e., transmembrane-spanning protein X, that is thermodynamically stable in a membrane.

As used herein, the phrase “a protein X cytoplasmic signaling domain,” for example, a CD3ζ cytoplasmic signaling domain, refers to any portion of a protein (protein X) that interacts with the interior of a cell or organelle and is capable of relaying a primary signal as known in the art, which lead to immune cell proliferation and/or activation. The cytoplasmic signaling domain as described herein differs from a co-stimulatory signaling domain, which relays a secondary signal for fully activating immune cells.

As used herein, the phrase “a protein X co-stimulatory signaling domain,” e.g., a CD28 co-stimulatory signaling domain, refers to the portion of a given co-stimulatory protein (protein X, such as CD28, 4-1BB, OX40, CD27, or ICOS) that can transduce co-stimulatory signals (secondary signals) into immune cells (such as T cells), leading to fully activation of the immune cells.

In some embodiments, CAR polypeptides described herein may further comprise a hinge domain, which may be located at the C-terminus of the antigen binding domain and the N-terminus of the transmembrane domain. The hinge may be of any suitable length. In other embodiments, the CAR polypeptide described herein may have no hinge domain at all. In yet other embodiments, the CAR polypeptide described herein may have a shortened hinge domain (e.g., including up to 25 amino acid residues).

As used in this specification and the appended claims, the singular forms “a,” “an,” and “the” include plural references unless the context clearly dictates otherwise.

A. Extracellular Antigen Binding Domains

The CAR polypeptides described herein comprise an extracellular antigen binding domain, which re-directs the specificity of immune cells expressing the CAR polypeptide. As used herein, “an extracellular antigen binding domain” refers to a peptide or polypeptide having binding specificity to a target antigen of interest (e.g., GPC3). The extracellular antigen binding domain as described herein does not comprise an extracellular domain of an Fc receptor, and may not bind to the Fc portion of an immunoglobulin. An extracellular domain that does not bind to an Fc fragment means that the binding activity between the two is not detectable using a conventional assay or only background or biologically insignificant binding activity is detected using the conventional assay.

In some instances, the extracellular antigen binding domain may be a single-chain antibody fragment (scFv), which may be derived from an antibody that binds the target cell surface antigen with a high binding affinity. The extracellular antigen binding domain may comprise an antigen binding fragment (e.g., a scFv) derived from a known anti-GPC3 antibody (e.g., Codrituzumab).

In some embodiments, the scFv comprises a heavy chain variable region comprising the amino acid sequence of:

(SEQ ID NO: 74)
QVQLVQSGAEVKKPGASVKVSCKASGYTFTDYEMHWVRQAPGQGLEWMGA
LDPKTGDTAYSQKFKGRVTLTADKSTSTAYMELSSLTSEDTAVYYCTRFY
SYTYWGQGTLV.

In some embodiments, the scFv comprises a light chain variable region comprising the amino acid sequence of:

(SEQ ID NO: 75)
DVVMTQSPLSLPVTPGEPASISCRSSQSLVHSNRNTYLHWYLQKPGQSPQ
LLIYKVSNRFSGVPDRFSGSGSGTDFTLKISRVEAEDVGVYYCSQNTHVP
PTFGQGTKLEI.

The extracellular antigen binding domain of any of the CAR polypeptides described herein may have suitable binding affinity for GPC3. As used herein, “binding affinity” refers to the apparent association constant or K_{A}. The K_{A} is the reciprocal of the dissociation constant (K_{D}). The extracellular antigen binding domain for use in the CAR polypeptides described herein may have a binding affinity (K_{D}) of at least 10⁻⁵, 10⁻⁶, 10⁻⁷, 10⁻⁸, 10⁻⁹, 10⁻¹⁹ M, or lower for the target antigen or antigenic epitope. An increased binding affinity corresponds to a decreased K_{D}. Higher affinity binding of an extracellular antigen binding domain for a first antigen relative to a second antigen can be indicated by a higher K_{A} (or a smaller numerical value K_{D}) for binding the first antigen than the K_{A} (or numerical value K_{D}) for binding the second antigen. In such cases, the extracellular antigen binding domain has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

B. Transmembrane Domain

The transmembrane domain of the CAR polypeptides described herein can be in any form known in the art. As used herein, a “transmembrane domain” refers to any protein structure that is thermodynamically stable in a cell membrane, preferably a eukaryotic cell membrane. A transmembrane domain compatible for use in the CAR polypeptides used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR polypeptide described herein is derived from a Type I single-pass membrane protein. Single-pass membrane proteins include, but are not limited to, CD8α, CD8β, 4-1BB/CD137, CD27, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, CD32, CD64, VEGFR2, FAS, and FGFR2B. In some examples, the transmembrane domain is of CD8 (e.g., the transmembrane domain is of CD8α). In some examples, the transmembrane domain is of 4-1BB/CD137. In other examples, the transmembrane domain is of CD28. In some instances, such a CAR polypeptide may be free of any hinge domain. Alternatively or in addition, such a CAR polypeptide may comprise two or more co-stimulatory regions as described herein. In other examples, the transmembrane domain is of CD34. In yet other examples, the transmembrane domain is not derived from human CD8α. In some embodiments, the transmembrane domain of the CAR polypeptide is a single-pass alpha helix.

Transmembrane domains from multi-pass membrane proteins may also be compatible for use in the CAR polypeptides described herein. Multi-pass membrane proteins may comprise a complex alpha helical structure (e.g., at least 2, 3, 4, 5, 6, 7 or more alpha helices) or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the CAR polypeptide described herein.

Transmembrane domains for use in the CAR polypeptides described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of each of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, hydrophobic or hydrophilic characteristics of a protein or protein segment, can be assessed by any method known in the art including, for example, the Kyte and Doolittle hydropathy analysis.

C. Co-Stimulatory Signaling Domains

Many immune cells require co-stimulation, in addition to stimulation of an antigen-specific signal, to promote cell proliferation, differentiation and survival, as well as to activate effector functions of the cell. In some embodiments, the CAR polypeptides described herein comprise at least one co-stimulatory signaling domain. In certain embodiments, the CAR polypeptides may contain a CD28 co-stimulatory signaling domain or a 4-1BB (CD137) co-stimulatory signaling domain. The term “co-stimulatory signaling domain,” as used herein, refers to at least a fragment of a co-stimulatory signaling protein that mediates signal transduction within a cell to induce an immune response such as an effector function (a secondary signal). As known in the art, activation of immune cells such as T cells often requires two signals: (1) the antigen specific signal (primary signal) triggered by the engagement of T cell receptor (TCR) and antigenic peptide/MHC complexes presented by antigen presenting cells, which typically is driven by CD3ζ as a component of the TCR complex; and (ii) a co-stimulatory signal (secondary signal) triggered by the interaction between a co-stimulatory receptor and its ligand. A co-stimulatory receptor transduces a co-stimulatory signal (secondary signal) as an addition to the TCR-triggered signaling and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CAR polypeptides described herein. The type(s) of co-stimulatory signaling domain is selected based on factors such as the type of the immune cells in which the CAR polypeptides would be expressed (e.g., T cells, NK cells, macrophages, neutrophils, or eosinophils) and the desired immune effector function. Examples of co-stimulatory signaling domains for use in the CAR polypeptides may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue mutations (e.g., 1, 2, 3, 4, 5, or 8) such as amino acid substitutions, deletions, or additions as compared to a wild-type counterpart. Such co-stimulatory signaling domains comprising one or more amino acid variations (e g, amino acid substitutions, deletions, or additions) may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the CAR polypeptide. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant.

In some embodiments, the CAR polypeptides may contain a single co-stimulatory domain such as, for example, a CD27 co-stimulatory domain, a CD28 co-stimulatory domain, a 4-1BB co-stimulatory domain, an ICOS co-stimulatory domain, or an OX40 co-stimulatory domain.

In some embodiments, the CAR polypeptides may comprise more than one co-stimulatory signaling domain (e.g., 2, 3, or more). In some embodiments, the CAR polypeptide comprises two or more of the same co-stimulatory signaling domains, for example, two copies of the co-stimulatory signaling domain of CD28. In some embodiments, the CAR polypeptide comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the CAR polypeptides (e.g., T cells or NK cells) and the desired immune effector function. In some embodiments, the CAR polypeptide comprises two co-stimulatory signaling domains, for example, two copies of the co-stimulatory signaling domain of CD28. In some embodiments, the CAR polypeptide may comprise two or more co-stimulatory signaling domains from different co-stimulatory receptors, such as any two or more co-stimulatory receptors described herein, for example, CD28 and 4-1BB, CD28 and CD27, CD28 and ICOS, CD28_{LL→GG} variant and 4-1BB, CD28 and OX40, or CD28_{LL→GG} variant and OX40. In some embodiments, the two co-stimulatory signaling domains are CD28 and 4-1BB. In some embodiments, the two co-stimulatory signaling domains are CD28_{LL→GG} variant and 4-1BB. In some embodiments, the two co-stimulatory signaling domains are CD28 and OX40. In some embodiments, the two co-stimulatory signaling domains are CD28_{LL→GG} variant and OX40. In some embodiments, the CAR constructs described herein may contain a combination of a CD28 and ICOSL. In some embodiments, the CAR construct described herein may contain a combination of CD28 and CD27. In certain embodiments, the 4-1BB co-stimulatory domain is located N-terminal to the CD28 or CD28_{LL→GG} variant co-stimulatory signaling domain.

In some embodiments, the CAR polypeptides described herein do not comprise a co-stimulatory signaling domain.

D. Cytoplasmic Signaling Domain

Any cytoplasmic signaling domain can be used to create the CAR polypeptides described herein. Such a cytoplasmic domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells.

The cytoplasmic domain described herein may comprise an immunoreceptor tyrosine-based activation motif (ITAM) domain or may be ITAM free. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways.

In some examples, the cytoplasmic signaling domain is of CD3ζ or FcεR1γ. In other examples, cytoplasmic signaling domain is not derived from human CD3ζ.

In one specific embodiment, several signaling domains can be fused together for additive or synergistic effect. Non-limiting examples of useful additional signaling domains include part or all of one or more of TCR Zeta chain, CD28, OX40/CD134, 4-1BB/CD137, FcεRIγ, ICOS/CD278, IL2R-beta/CD122, IL-2R-gamma/CD132, and CD40.

In other embodiments, the cytoplasmic signaling domain described herein is free of the ITAM motif. Examples include, but are not limited to, the cytoplasmic signaling domain of Jak/STAT, Toll-interleukin receptor (TIR), and tyrosine kinase.

E. Hinge Domain

In some embodiments, the CAR polypeptides described herein further comprise a hinge domain that is located between the extracellular antigen-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain of the CAR polypeptide can be used.

Hinge domains of any protein known in the art to comprise a hinge domain are compatible for use in the CAR polypeptides described herein. In some embodiments, the hinge domain is at least a portion of a hinge domain of a naturally occurring protein and confers flexibility to the CAR polypeptide. In some embodiments, the hinge domain is of CD8. In some embodiments, the hinge domain is a portion of the hinge domain of CD8, e.g., a fragment containing at least 15 (e.g., 20, 25, 30, 35, or 40) consecutive amino acids of the hinge domain of CD8. In some embodiments, the hinge domain is of CD28. In some embodiments, the hinge domain is a portion of the hinge domain of CD28, e.g., a fragment containing at least 15 (e.g., 20, 25, 30, 35, or 40) consecutive amino acids of the hinge domain of CD28.

Hinge domains of antibodies, such as an IgG, IgA, IgM, IgE, or IgD antibodies, are also compatible for use in the CAR polypeptides described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the CAR polypeptides described herein. In some embodiments, the hinge domain between the C-terminus of the extracellular antigen-binding domain and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ(SEQ ID NO:3), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In certain embodiments, n can be an integer greater than 60. In some embodiments, the hinge domain is (Gly₄Ser)₃ (SEQ ID NO: 4). In some embodiments, the hinge domain is (Gly₄Ser)₆ (SEQ ID NO: 5). In some embodiments, the hinge domain is (Gly₄Ser)₉ (SEQ ID NO: 6). In some embodiments, the hinge domain is (Gly₄Ser)₁₂ (SEQ ID NO: 7). In some embodiments, the hinge domain is (Gly₄Ser)₁₅ (SEQ ID NO: 8). In some embodiments, the hinge domain is (Gly₄Ser)₃₀ (SEQ ID NO: 9). In some embodiments, the hinge domain is (Gly₄Ser)₄₅ (SEQ ID NO: 10). In some embodiments, the hinge domain is (Gly₄Ser)₆₀ (SEQ ID NO: 11).

In other embodiments, the hinge domain is an extended recombinant polypeptide (XTEN), which is an unstructured polypeptide consisting of hydrophilic residues of varying lengths (e.g., 10-80 amino acid residues) Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

Any of the hinge domains used for making the CAR polypeptide as described herein may contain up to 250 amino acid residues. In some instances, the CAR polypeptide may contain a relatively long hinge domain, for example, containing 150-250 amino acid residues (e.g., 150-180 amino acid residues, 180-200 amino acid residues, or 200-250 amino acid residues). In other instances, the CAR polypeptide may contain a medium sized hinge domain, which may contain 60-150 amino acid residues (e.g., 60-80, 80-100, 100-120, or 120-150 amino acid residues). Alternatively, the CAR polypeptide may contain a short hinge domain, which may contain less than 60 amino acid residues (e.g., 1-30 amino acids or 31-60 amino acids). In some embodiments, a CAR construct described herein contains no hinge domain.

F. Signal Peptide

In some embodiments, the CAR polypeptide also comprises a signal peptide (also known as a signal sequence) at the N-terminus of the polypeptide. In general, signal sequences are peptide sequences that target a polypeptide to the desired site in a cell. In some embodiments, the signal sequence targets the CAR polypeptide to the secretory pathway of the cell and will allow for integration and anchoring of the CAR polypeptide into the lipid bilayer. Signal sequences including signal sequences of naturally occurring proteins or synthetic, non-naturally occurring signal sequences that are compatible for use in the CAR polypeptides described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16.

G. Examples of CAR Polypeptides

Table 1 provides exemplary CAR polypeptides described herein. These exemplary constructs have, from N-terminus to C-terminus in order, the signal sequence, the antigen binding domain (e.g., a scFv fragment specific to GPC3), the hinge domain, and the transmembrane, while the positions of the optional co-stimulatory domain and the cytoplasmic signaling domain can be switched.

TABLE 1
Exemplary Components of CAR polypeptides.

| | Extracellular | | | Co- | |
|---|---|---|---|---|---|
| | domain | | Transmem- | stimu- | Cytoplasmic |
| Signal | (antigen | Hinge | brane | latory | Signaling |
| Sequence | binding) | domain | domain | domain | domain |
| CD8α | scFv (e.g., | CD8 | CD8 | 4-1BB | CD3ζ |
| | anti-GPC3 | | | | |
| | scFv) | | | | |
| CD8α | scFv (e.g., | CD28 | CD28 | CD28 | CD3ζ |
| | anti-GPC3 | | | | |
| | scFv) | | | | |

Amino acid sequences of the example CAR polypeptides are provided below (signal sequence italicized).

SEQ ID NO: 1:
MALPVTALLLPLALLLHAARPDVVMTQSPLSLPVTPGEPASISCRSSQSL
VHSNRNTYLHWYLQKPGQSPQLLIYKVSNRFSGVPDRFSGSGSGTDFTLK
ISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGAL
DPKTGDTAYSQKFKGRVTLTADKSTSTAYMELSSLTSEDTAVYYCTREYS
YTYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 2:
MALPVTALLLPLALLLHAARPDVVMTQSPLSLPVTPGEPASISCRSSQSL
VHSNRNTYLHWYLQKPGQSPQLLIYKVSNRFSGVPDRFSGSGSGTDFTLK
ISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGAL
DPKTGDTAYSQKFKGRVTLTADKSTSTAYMELSSLTSEDTAVYYCTREYS
YTYWGQGTLVTVSSIEVMYPPPYLDNEKSNGTIIHVKGKHLCPSPLFPGP
SKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR

### III. Hematopoietic Cells Expressing Co-Stimulatory Polypeptides and Anti-GPC3 CAR Polypeptides

Provided herein are genetically engineered host cells (e.g., hematopoietic cells such as hematopoietic stem cells and immune cells, e.g., T cells or NK cells) expressing one or more of the co-stimulatory polypeptides as described herein and an anti-GPC3 CAR polypeptides (CAR-expressing cells, e.g., CAR T cells) as also described herein. In some embodiments, the host cells are hematopoietic cells or a progeny thereof. In some embodiments, the hematopoietic cells can be hematopoietic stem cells. In other embodiments, the host cells are immune cells, such as T cells or NK cells. In some embodiments, the immune cells are T cells. In some embodiments, the immune cells are NK cells. In other embodiments, the immune cells can be established cell lines, for example, NK-92 cells.

In some instances, the co-stimulatory polypeptide to be introduced into the host cells is identical to an endogenous protein of the host cell. Introducing additional copies of the coding sequences of the co-stimulatory polypeptide into the host cell would enhance the expression level of the polypeptide (i.e., over-express) as relative to the native counterpart. In some instances, the co-stimulatory polypeptide to be introduced into the host cells is heterologous to the host cell, i.e., does not exist or is not expressed in the host cell. Such a heterologous co-stimulatory polypeptide may be a naturally-occurring protein not expressed in the host cell in nature (e.g., from a different species). Alternatively, the heterologous co-stimulatory polypeptide may be a variant of a native protein, such as those described herein. In some examples, the exogenous (i.e., not native to the host cells) copy of the coding nucleic acid may exist extrachromosomally. In other examples, the exogenous copy of the coding sequence may be integrated into the chromosome of the host cell, and may be located at a site that is different from the native loci of the endogenous gene.

Such genetically engineered host cells have the capacity to have a modulated co-stimulatory pathway. Given their expected high proliferation rate, bioactivity, and/or survival rate, the genetically engineered cells such as T cell and NK cells would be expected to have higher therapeutic efficacy as relative to CAR T cells that do not express or express a lower level or less active form of the co-stimulatory polypeptide.

The population of immune cells can be obtained from any source, such as peripheral blood mononuclear cells (PBMCs), bone marrow, or tissues such as spleen, lymph node, thymus, stem cells, or tumor tissue. Alternatively, the immune cell population may be derived from stem cells, for example, hematopoietic stem cells and induced pluripotent stem cells (iPSCs). A source suitable for obtaining the type of host cells desired would be evident to one of skill in the art. In some embodiments, the population of immune cells is derived from PBMCs, which may be obtained from a patient (e.g., a human patient) who needs the treatment described herein. The type of host cells desired (e.g., T cells, NK cells, or T cells and NK cells) may be expanded within the population of cells obtained by co-incubating the cells with stimulatory molecules. As a non-limiting example, anti-CD3 and anti-CD28 antibodies may be used for expansion of T cells.

To construct the immune cells that express any of the co-stimulatory polypeptides and the anti-GPC3 polypeptide described herein, expression vectors for stable or transient expression of the co-stimulatory polypeptides and/or the CAR polypeptide may be created via conventional methods as described herein and introduced into immune host cells. For example, nucleic acids encoding the co-stimulatory polypeptides and/or the CAR polypeptides may be cloned into one or two suitable expression vectors, such as a viral vector or a non-viral vector in operable linkage to a suitable promoter. In some instances, each of the coding sequences for the CAR polypeptide and the co-stimulatory polypeptide are on two separate nucleic acid molecules and can be cloned into two separate vectors, which may be introduced into suitable host cells simultaneously or sequentially. Alternatively, the coding sequences for the CAR polypeptide and the co-stimulatory polypeptide are on one nucleic acid molecule and can be cloned into one vector. The coding sequences of the CAR polypeptide and the co-stimulatory polypeptide may be in operable linkage to two distinct promoters such that the expression of the two polypeptides is controlled by different promoters. Alternatively, the coding sequences of the CAR polypeptide and the co-stimulatory polypeptide may be in operable linkage to one promoter such that the expression of the two polypeptides is controlled by a single promoter. Suitable sequences may be inserted between the coding sequences of the two polypeptides so that two separate polypeptides can be translated from a single mRNA molecule. Such sequences, for example, IRES or ribosomal skipping site, are well known in the art. Additional descriptions are provided below.

The nucleic acids and the vector(s) may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the co-stimulatory polypeptides and/or the anti-GPC3 CAR polypeptides. The synthetic linkers may contain nucleic acid sequences that correspond to a particular restriction site in the vector. The selection of expression vectors/plasmids/viral vectors would depend on the type of host cells for expression of the co-stimulatory polypeptides and/or the CAR polypeptides, but should be suitable for integration and replication in eukaryotic cells.

A variety of promoters can be used for expression of the co-stimulatory polypeptides and/or the anti-GPC3 CAR polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, the human EF1-alpha promoter, or herpes simplex tk virus promoter. Additional promoters for expression of the co-stimulatory polypeptides and/or the CAR polypeptides include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; intron sequences of the human EF1-alpha gene; transcription termination and RNA processing signals from SV40 for mRNA stability; SV40 polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the co-stimulatory polypeptides and/or the anti-GPC3 CAR polypeptide.

In one specific embodiment, such vectors also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, nitroreductase, and caspases such as caspase 8.

Suitable vectors and methods for producing vectors containing transgenes are well known and available in the art. Examples of the preparation of vectors for expression of co-stimulatory polypeptides and/or anti-GPC3 CAR polypeptides can be found, for example, in US2014/0106449, herein incorporated in its entirety by reference.

Any of the vectors comprising a nucleic acid sequence that encodes a co-stimulatory polypeptide and/or an anti-GPC3 CAR polypeptide described herein is also within the scope of the present disclosure. Such a vector, or the sequence encoding a co-stimulatory polypeptide and/or a CAR polypeptide contained therein, may be delivered into host cells such as host immune cells by any suitable method. Methods of delivering vectors to immune cells are well known in the art and may include DNA electroporation, RNA electroporation, transfection using reagents such as liposomes, or viral transduction (e.g., retroviral transduction such as lentiviral transduction).

In some embodiments, the vectors for expression of the co-stimulatory polypeptides and/or the anti-GPC3 CAR polypeptides are delivered to host cells by viral transduction (e.g., retroviral transduction such as lentiviral or gammaretroviral transduction). Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; and WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984; and WO 95/00655). In some embodiments, the vectors for expression of the co-stimulatory polypeptides and/or the CAR polypeptides are retroviruses. In some embodiments, the vectors for expression of the co-stimulatory polypeptides and/or the CAR polypeptides are lentiviruses.

Examples of references describing retroviral transduction include Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993). WO 95/07358 describes high efficiency transduction of primary B lymphocytes. See also WO2016040441A1, which is incorporated by reference herein for the purpose and subject matter referenced herein.

In examples in which the vectors encoding co-stimulatory polypeptides and/or anti-GPC3 CAR polypeptides are introduced to the host cells using a viral vector, viral particles that are capable of infecting the immune cells and carry the vector may be produced by any method known in the art and can be found, for example in WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

In some embodiments, RNA molecules encoding any of the co-stimulatory polypeptides and/or the anti-GPC3 CAR polypeptides as described herein may be prepared by a conventional method (e.g., in vitro transcription) and then introduced into suitable host cells, e.g., those described herein, via known methods, e.g., Rabinovich et al., Human Gene Therapy 17:1027-1035.

In some instances, the nucleic acid encoding a co-stimulatory polypeptide and the nucleic acid encoding a suitable anti-GPC3 CAR polypeptide may be cloned into separate expression vectors, which may be introduced into suitable host cells concurrently or sequentially. For example, an expression vector (or an RNA molecule) for expressing the co-stimulatory polypeptide may be introduced into host cells first and transfected host cells expressing the co-stimulatory polypeptide may be isolated and cultured in vitro. An expression vector (or an RNA molecule) for expressing a suitable CAR polypeptide can then introduced into the host cells that express the co-stimulatory polypeptide and transfected cells expressing both polypeptides can be isolated. In another example, expression vectors (or RNA molecules) each for expressing the co-stimulatory polypeptide and the CAR polypeptide can be introduced into host cells simultaneously and transfected host cells expressing both polypeptides can be isolated via routine methodology.

In other instances, the nucleic acid encoding the co-stimulatory polypeptide and the nucleic acid encoding the anti-GPC3 CAR polypeptide may be cloned into the same expression vector. Polynucleotides (including vectors in which such polynucleotides are operably linked to at least one regulatory element) for expression of the CAR and co-stimulatory polypeptide are also within the scope of the present disclosure. Non-limiting examples of useful vectors of the disclosure include viral vectors such as, e.g., retroviral vectors including gamma retroviral vectors, adeno-associated virus vectors (AAV vectors), and lentiviral vectors.

In some instances, the nucleic acid(s) encoding the co-stimulatory polypeptide and/or the anti-GPC3 CAR polypeptide may be delivered into host cells via transposons. In some instances, the encoding nucleic acid(s) may be delivered into host cells via gene editing, for example, by CRISPR, TALEN, ZFN, or meganucleases.

In some instances, the nucleic acid described herein may comprise two coding sequences, one encoding an anti-GPC3 CAR polypeptide as described herein, and the other encoding a polypeptide capable of modulating a co-stimulatory pathway (i.e., a co-stimulatory polypeptide). The nucleic acid comprising the two coding sequences described herein may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a P2A, T2A, or F2A peptide (see, for example, Kim et al., PLoS One. 2011; 6(4):e18556). As a non-limiting example, an exemplary P2A peptide may have the amino acid sequence of ATNFSLLKQAGDVEENPGP SEQ ID NO: 72.

In another embodiment, the third nucleotide sequence may encode an internal ribosome entry site (IRES). An IRES is an RNA element that allows translation initiation in an end-independent manner, also permitting the translation of additional open reading frames from one messenger RNA. Alternatively, the third nucleotide sequence may encode a second promoter controlling the expression of the second polypeptide. The third nucleotide sequence may also encode more than one ribosomal skipping sequence, IRES sequence, additional promoter sequence, or a combination thereof.

The nucleic acid may also include additional coding sequences (including, but not limited to, fourth and fifth coding sequences) and may be configured such that the polypeptides encoded by the additional coding sequences are expressed as further independent and physically separate polypeptides. To this end, the additional coding sequences may be separated from other coding sequences by one or more nucleotide sequences encoding one or more ribosomal skipping sequences, IRES sequences, or additional promoter sequences.

In some examples, the nucleic acid (e.g., an expression vector or an RNA molecule as described herein) may comprise coding sequences for both the co-stimulatory polypeptide (e.g., those described herein) and a suitable anti-GPC3 CAR polypeptide, the two coding sequences, in any order, being separated by a third nucleotide sequence coding for a P2A peptide (e.g., ATNFSLLKQAGDVEENPGP; SEQ ID NO: 72). As a result, two separate polypeptides, the co-stimulatory polypeptide and the CAR, can be produced from such a nucleic acid, wherein the P2A portion ATNFSLLKQAGDVEENPG (SEQ ID NO: 73) is linked to the upstream polypeptide (encoded by the upstream coding sequence) and residue P from the P2A peptide is linked to the downstream polypeptide (encoded by the downstream coding sequence). In some examples, the CAR polypeptide is the upstream one and the co-stimulatory polypeptide is the downstream one. In other examples, the co-stimulatory polypeptide is the upstream one and the CAR polypeptide is the downstream one.

In some examples, the nucleic acid described above may further encode a linker (e.g., a GSG linker) between two segments of the encoded sequences, for example, between the upstream polypeptide and the P2A peptide.

In specific examples, the nucleic acid described herein is configured such that it expresses two separate polypeptides in the host cell to which the nucleic acid is transfected: (i) the first polypeptide that contains, from the N-terminus to the C-terminus, a suitable anti-GPC3 CAR (e.g., SEQ ID NO:1 or SEQ ID NO:2), a peptide linker (e.g., the GSG linker), and the ATNFSLLKQAGDVEENPG (SEQ ID NO: 73) segment derived from the P2A peptide; and (ii) a second polypeptide that contains, from the N-terminus to the C-terminus, the P residue derived from the P2A peptide and the co-stimulatory polypeptide (e.g., any of SEQ ID NOs: 12-71).

In some examples, the genetically engineered immune cells co-express the anti-GPC3 CAR in combination with a co-stimulatory polypeptide such as 4-1BB, 4-1BBL (e.g., a variant of a native 4-1BBL such as those described herein), ICOS, ICOSL, OX40, OX40L, CD70, LIGHT, CD30L, GITRL, CD40, CD40L, TL1A, BAFFR, or CD27. In other examples, the genetically engineered immune cells co-express the CAR construct in combination with a co-stimulatory polypeptide such as 4-1BBL (e.g., a variant of a native 4-1BBL such as those described herein), ICOSL, OX40L, CD70, LIGHT, GITRL, CD40L, or TL1A. Alternatively, the genetically engineered immune cells may co-express a CAR comprising a CD28 co-stimulatory domain in combination with a co-stimulatory polypeptide that also comprises a CD28 co-stimulatory domain.

In some embodiments, the CAR polypeptide comprises a co-stimulatory domain of a CD28 co-stimulatory molecule, and the co-stimulatory polypeptide is CD70, LIGHT, OX40L, TL1A, BAFFR, CD40, CD40L, CD27, 4-1BB, or ICOS. In some embodiments, the CAR polypeptide comprises a co-stimulatory domain of a CD28 co-stimulatory molecule, and the co-stimulatory polypeptide is BAFFR or CD27. The CD28 co-stimulatory molecule may comprises the amino acid sequence of SEQ ID NO: 12. The BAFFR may comprise the amino acid sequence of SEQ ID NO: 31, and the CD27 may comprise the amino acid sequence of SEQ ID NO: 33.

In other embodiments, the CAR polypeptide comprises a co-stimulatory domain of a 4-1BB co-stimulatory molecule, and the co-stimulatory polypeptide is CD70, LIGHT, OX40L, BAFFR, CD27, or OX40. In other embodiments, the CAR polypeptide comprises a co-stimulatory domain of a 4-1BB co-stimulatory molecule, and the co-stimulatory polypeptide is CD70, LIGHT, or OX40L. The 4-1BB co-stimulatory molecule may comprise the amino acid sequence of SEQ ID NO: 22. The CD70 may comprise the amino acid sequence of SEQ ID NO: 34, the LIGHT may comprise the amino acid sequence of SEQ ID NO: 43, and the OX40L may comprise the amino acid sequence of SEQ ID NO: 47.

In other embodiments, the genetically engineered immune cells co-express an anti-GPC3 CAR with a 4-1BB costimulatory domain such as SEQ ID NO: 1 in combination with a co-stimulatory polypeptide such as 4-1BB, 4-1BBL (e.g., a variant of a native 4-1BBL such as those described herein), ICOS, ICOSL, OX40, OX40L, CD70, LIGHT, CD30L, GITRL, CD40, CD40L, TL1A, BAFFR (e.g., a variant of a native BAFFR such as those described herein), or CD27. In some embodiments, the genetically engineered immune cells co-express an anti-GPC3 CAR with a 4-1BB costimulatory domain such as SEQ ID NO: 1 in combination with a co-stimulatory polypeptide of ICOSL, BAFFR (e.g., a variant of a native BAFFR such as those described herein), LIGHT, CD30L, or CD27.

In yet other embodiments, the genetically engineered immune cells co-express an anti-GPC3 CAR with a CD28 costimulatory domain such as SEQ ID NO: 2 in combination of a co-stimulatory polypeptide such as 4-1BB, 4-1BBL (e.g., a variant of a native 4-1BBL such as those described herein), ICOS, ICOSL, OX40, OX40L, CD70, LIGHT, CD30L, GITRL, CD40, CD40L, TL1A, BAFFR (e.g., a variant of a native BAFFR such as those described herein), or CD27. In some embodiments, the genetically engineered immune cells co-express an anti-GPC3 CAR with a CD28 costimulatory domain such as SEQ ID NO: 2 in combination with a co-stimulatory polypeptide of ICOSL, BAFFR (e.g., a variant of a native BAFFR such as those described herein), LIGHT, CD30L, or CD27.

Alternatively, the genetically engineered immune cells may co-express a CAR comprising a co-stimulatory domain such as 4-1BB or CD28 in combination with a co-stimulatory polypeptide that also comprises the same co-stimulatory domain. In other embodiments, the genetically engineered immune cells may co-express a CAR comprising a co-stimulatory domain such as 4-1BB or CD28 in combination with a different co-stimulatory polypeptide, for example, 4-1BB, 4-1BBL (e.g., a variant of a native 4-1BBL such as those described herein), ICOS, ICOSL, OX40, OX40L, CD70, LIGHT, CD30L, GITRL, CD40, CD40L, TL1A, BAFFR, or CD27.

In some embodiments, the genetically engineered immune cells may co-express a CAR comprising co-stimulatory domain such as 4-1BB or CD28 and a hinge domain in combination with a co-stimulatory polypeptide that also comprises a co-stimulatory domain. In some embodiments, the co-stimulatory domain, hinge domain, and co-stimulatory polypeptide are from the same co-stimulatory molecule, such as 4-1BB or CD28. In some embodiments, the co-stimulatory domain, hinge domain, and co-stimulatory polypeptide are from the different co-stimulatory molecules. Alternatively or in addition, the CAR construct disclosed herein may comprise a transmembrane domain of CD8 or a portion thereof.

In some embodiments, the genetically engineered immune cells may co-express a CAR that is free of any hinge domain in combination with a co-stimulatory polypeptide, e.g., 4-1BB, 4-1BBL (e.g., a variant of a native 4-1BBL such as those described herein), ICOS, ICOSL, OX40, OX40L, CD70, LIGHT, CD30L, GITRL, CD40, CD40L, TL1A, BAFFR (e.g., a variant of a native BAFFR such as those described herein), or CD27. In some embodiments, the genetically engineered immune cells co-express a CAR that is free of any hinge domain in combination with a co-stimulatory polypeptide of ICOSL, BAFFR (e.g., a variant of a native BAFFR such as those described herein), LIGHT, CD30L, or CD27.

In some embodiments, the genetically engineered immune cells may co-express a CAR (e.g., those described herein) and a co-stimulatory polypeptide, which is 4-1BBL. In some instances, the 4-1BBL can be a functional variant of a naturally occurring 4-1BBL (e.g., human 4-1BBL), for example, any of the variants disclosed herein (e.g., 4-1BBL Q89A, 4-1BBL L115A, 4-1BBL K127A, or 4-1BBL Q227A). In some examples, the 4-1BBL polypeptide is a truncated variant of a naturally occurring counterpart, wherein the truncated variant lacks the cytoplasmic fragment.

In some embodiments, the genetically engineered immune cells (e.g., T cells) co-express (a) a CAR construct comprising a 4-1BB co-stimulatory domain (e.g., SEQ ID NO:1) or a CD28-co-stimulatory domain (e.g., SEQ ID NO:2), and (b) a co-stimulatory molecule (exogenous) as those disclosed herein (e.g., CD70, LIGHT, OX40L, or CD27), and exhibit higher bioactivity (which may be evidenced by higher IL-2 secretion), and/or higher proliferation activity, as relative to immune cells expressing the same CAR but not the exogenous co-stimulatory molecule. In some embodiments, the genetically engineered immune cells (e.g., T cells) co-express: (a) a CAR construct comprising anti-GPC3 CAR with a 4-1BB costimulatory domain (for example, a CAR construct comprising SEQ ID NO: 1), and (b) CD70. In some embodiments, the genetically engineered immune cells (e.g., T cells) co-express: (a) a CAR construct comprising anti-GPC3 CAR with a 4-1BB co-stimulatory domain (for example, a CAR construct comprising SEQ ID NO: 1), and (b) LIGHT. In some embodiments, the genetically engineered immune cells (e.g., T cells) co-express: (a) a CAR construct comprising anti-GPC3 CAR with a 4-1BB co-stimulatory domain (for example, a CAR construct comprising SEQ ID NO: 1), and (b) OX40L. In some embodiments, the genetically engineered immune cells (e.g., T cells) co-express: (a) a CAR construct comprising a CD28 co-stimulatory domain (for example, a CAR construct comprising SEQ ID NO: 2), and (b) CD27.

As the examples below show, when expressed with their co-stimulatory molecules, such CAR constructs exhibit: improved proliferation; improved cytokine production; improved efficacy in in vivo mouse tumor models; increased T cell persistence; improved resistence to MDSC suppression; and/or improved resistance to Treg suppression relative to their respective parental CAR constructs. In some instances, additional polypeptides of interest may also be introduced into the host immune cells.

Following introduction into the host cells a vector encoding any of the co-stimulatory polypeptides and/or the anti-GPC3 CAR polypeptides provided herein, or the nucleic acid encoding the anti-GPC3 CAR polypeptide and/or co-stimulatory polypeptide (e.g., an RNA molecule), the cells may be cultured under conditions that allow for expression of the co-stimulatory polypeptide and/or the CAR polypeptide. In examples in which the nucleic acid encoding the co-stimulatory polypeptide and/or the CAR polypeptide is regulated by a regulatable promoter, the host cells may be cultured in conditions wherein the regulatable promoter is activated. In some embodiments, the promoter is an inducible promoter and the immune cells are cultured in the presence of the inducing molecule or in conditions in which the inducing molecule is produced. Determining whether the co-stimulatory polypeptide and/or the CAR polypeptide is expressed will be evident to one of skill in the art and may be assessed by any known method, for example, detection of the co-stimulatory polypeptide and/or the CAR polypeptide-encoding mRNA by quantitative reverse transcriptase PCR (qRT-PCR) or detection of the co-stimulatory polypeptide and/or the CAR polypeptide protein by methods including Western blotting, fluorescence microscopy, and flow cytometry.

Alternatively, expression of the anti-GPC3 CAR polypeptide may take place in vivo after the immune cells are administered to a subject. As used herein, the term “subject” refers to any mammal such as a human, monkey, mouse, rabbit, or domestic mammal. For example, the subject may be a primate. In a preferred embodiment, the subject is human.

Alternatively, expression of a co-stimulatory polypeptide and/or an anti-GPC3 polypeptide in any of the immune cells disclosed herein can be achieved by introducing RNA molecules encoding the co-stimulatory polypeptides and/or the CAR polypeptides. Such RNA molecules can be prepared by in vitro transcription or by chemical synthesis. The RNA molecules can then be introduced into suitable host cells such as immune cells (e.g., T cells, NK cells, or both T cells and NK cells) by, e.g., electroporation. For example, RNA molecules can be synthesized and introduced into host immune cells following the methods described in Rabinovich et al., Human Gene Therapy, 17:1027-1035 and WO WO2013/040557.

In certain embodiments, a vector(s) or RNA molecule(s) comprising the co-stimulatory polypeptide and/or the anti-GPC3 CAR polypeptide may be introduced to the host cells or immune cells in vivo. As a non-limiting example, this may be accomplished by administering a vector or RNA molecule encoding one or more co-stimulatory polypeptides and/or one or more CAR polypeptides described herein directly to the subject (e.g., through intravenous administration), producing host cells comprising co-stimulatory polypeptides and/or CAR polypeptides in vivo.

Methods for preparing host cells expressing any of the co-stimulatory polypeptides and/or the anti-GPC3 CAR polypeptides described herein may also comprise activating the host cells ex vivo. Activating a host cell means stimulating a host cell into an activated state in which the cell may be able to perform effector functions (e.g., cytotoxicity). Methods of activating a host cell will depend on the type of host cell used for expression of the co-stimulatory polypeptides and/or CAR polypeptides. For example, T cells may be activated ex vivo in the presence of one or more molecules including, but not limited to: an anti-CD3 antibody, an anti-CD28 antibody, IL-2, phytohemagglutinin, engineered artificial stimulatory cells or particles, or a combination thereof. The engineered artificial stimulatory cells may be artificial antigen-presenting cells as known in the art. See, e.g., Neal et al., J. Immunol. Res. Ther. 2017, 2(1):68-79 and Turtle et al., Cancer J. 2010, 16(4):374-381, the relevant disclosures of each of which are hereby incorporated by reference for the purpose and subject matter referenced herein.

In other examples, NK cells may be activated ex vivo in the presence of one or more molecules such as a 4-1BB ligand, an anti-4-1BB antibody, IL-15, an anti-IL-15 receptor antibody, IL-2, IL12, IL-18, IL-21, K562 cells, and/or engineered artificial stimulatory cells or particles. In some embodiments, the host cells expressing any of the co-stimulatory polypeptides and/or the anti-GPC3 CAR polypeptides (CAR- and/or co-stimulatory polypeptide-expressing cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

Methods for preparing host cells expressing any of the co-stimulatory polypeptides and/or the anti-GPC3 CAR polypeptides described herein may comprise expanding the host cells ex vivo. Expanding host cells may involve any method that results in an increase in the number of cells expressing co-stimulatory polypeptides and/or CAR polypeptides, for example, allowing the host cells to proliferate or stimulating the host cells to proliferate. Methods for stimulating expansion of host cells will depend on the type of host cell used for expression of the co-stimulatory polypeptides and/or the CAR polypeptides and will be evident to one of skill in the art. In some embodiments, the host cells expressing any of the co-stimulatory polypeptides and/or the CAR polypeptides described herein are expanded ex vivo prior to administration to a subject.

In some embodiments, the host cells expressing the co-stimulatory polypeptides and/or the anti-GPC3 CAR polypeptides are expanded and activated ex vivo prior to administration of the cells to the subject. Host cell activation and expansion may be used to allow integration of a viral vector into the genome and expression of the gene encoding a co-stimulatory polypeptide and/or an anti-GPC3 CAR polypeptide as described herein. If mRNA electroporation is used, no activation and/or expansion may be required, although electroporation may be more effective when performed on activated cells. In some instances, a co-stimulatory polypeptide and/or a CAR polypeptide is transiently expressed in a suitable host cell (e.g., for 3-5 days). Transient expression may be advantageous if there is a potential toxicity and should be helpful in initial phases of clinical testing for possible side effects.

Any of the host cells expressing the co-stimulatory polypeptides and/or the anti-GPC3 CAR polypeptides may be mixed with a pharmaceutically acceptable carrier to form a pharmaceutical composition, which is also within the scope of the present disclosure.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

The pharmaceutical compositions of the disclosure may also contain one or more additional active compounds as necessary for the particular indication being treated, preferably those with complementary activities that do not adversely affect each other. Non-limiting examples of possible additional active compounds include, e.g., IL-2 as well as various agents known in the field and listed in the discussion of combination treatments, below.

### IV. Immunotherapy Using the Genetically Engineered Hematopoietic Cells Described Herein

The genetically engineered host cells such as hematopoietic cells, for example, immune cells described herein, co-expressing a co-stimulatory polypeptide and an anti-GPC3 CAR polypeptide can be used in immune therapy such as T-cell therapy or NK-cell therapy for inhibiting diseased cells expressing an antigen to which the CAR polypeptide targets, directly or indirectly (e.g., via a therapeutic agent conjugated to a tag to which the CAR polypeptide binds). The co-stimulatory polypeptide co-expressed with a CAR polypeptide in immune cells would facilitate the cell-based immune therapy by allowing the cells to grow and/or function effectively in a low glucose, low amino acid, low pH, and/or a hypoxic environment, for example, in a tumor microenvironment. Clinical safety may be further enhanced by using mRNA electroporation to express the co-stimulatory polypeptide and/or the CAR polypeptide transiently, to limit any potential non-tumor specific reactivity.

The methods described herein may comprise introducing into the subject a therapeutically effective amount of genetically engineered host cells such as immune cells (e.g., T lymphocytes or NK cells), which co-express a co-stimulatory polypeptide and a CAR polypeptide of the disclosure. The subject (e.g., a human patient such as a human cancer patient) may additionally have been treated or is being treated with an anti-cancer therapy including, but not limited to, an anti-cancer therapeutic agent.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a pharmaceutical composition comprising a population of T lymphocytes or NK cells that express a co-stimulatory polypeptide and/or a chimeric antigen receptor (CAR) construct and an additional anti-cancer therapeutic), the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

A. Enhancing Efficacy of Cell-Based Immune Therapy

Host cells (e.g., immune cells such as T cells and NK cells) expressing co-stimulatory polypeptides and anti-GPC3 CAR polypeptides described herein are useful for inhibiting cells expressing a target antigen and/or for enhancing growth and/or proliferation of immune cells in a low-glucose environment, a low amino acid environment, a low pH environment, and/or a hypoxic environment, for example, in a tumor microenvironment. In some embodiments, the subject is a mammal, such as a human, monkey, mouse, rabbit, or domestic mammal. In some embodiments, the subject is a human. In some embodiments, the subject is a human cancer patient. In some embodiments, the subject has additionally been treated or is being treated with any of the therapeutic antibodies described herein.

To practice the method described herein, an effective amount of the immune cells (NK cells and/or T lymphocytes) expressing any of the co-stimulatory polypeptides and the CAR polypeptides described herein, or compositions thereof may be administered to a subject in need of the treatment via a suitable route, such as intravenous administration. As used herein, an effective amount refers to the amount of the respective agent (e.g., the NK cells and/or T lymphocytes expressing co-stimulatory polypeptides, CAR polypeptides, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject associated with GPC3⁺ cells. In some embodiments, the subject is a human. In some embodiments, the subject in need of treatment is a human cancer patient.

The methods of the disclosure may be used for treatment of any cancer or any pathogen. Specific non-limiting examples of cancers which can be treated by the methods of the disclosure include, for example, breast cancer, gastric cancer, lung cancer, skin cancer, prostate cancer, colorectal cancer, renal cell carcinoma, ovarian cancer, rhabdomyosarcoma, germ cell cancer, hepatoblastoma, mesothelioma, pancreatic cancer, head and neck cancer, glioma, glioblastoma, thyroid cancer, hepatocellular cancer, esophageal cancer, and cervical cancer. In certain embodiments, the cancer may be a solid breast cancer, lung cancer, or hepatocellular cancer. In certain embodiments, the cancer may be a solid tumor.

The methods of this disclosure may also be used for treating infectious diseases, which may be caused by bacterial infection, viral infection, or fungus infection. In such instances, the genetically engineered immune cells can be co-used with an Fc-containing therapeutic agent (e.g., an antibody) that targets a pathogenic antigen (e.g., an antigen associated with the bacterium, virus, or fungus that causes the infection). Specific non-limiting examples of pathogenic antigens include, but are not limited to, bacterial, viral, and/or fungal antigens. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, or human hepatitis C virus glycoprotein E1 or E2.

In some embodiments, the immune cells are administered to a subject in an amount effective in inhibiting cells expressing GPC3 by least 20% and/or by at least 2-fold, e.g., inhibiting cells expressing the target antigen by 50%, 80%, 100%, 2-fold, 5-fold, 10-fold, 20-fold, 50-fold, 100-fold, or more.

Additional therapeutic agents (e.g., antibody-based immunotherapeutic agents) may be used to treat, alleviate, or reduce the symptoms of any disease or disorder for which the therapeutic agent is considered useful in a subject.

The efficacy of the cell-based immunotherapy as described herein may be assessed by any method known in the art and would be evident to a skilled medical professional. For example, the efficacy of the cell-based immunotherapy may be assessed by survival of the subject or tumor or cancer burden in the subject or tissue or sample thereof. In some embodiments, the immune cells are administered to a subject in need of the treatment in an amount effective in enhancing the efficacy of a cell-based immunotherapy by at least 20% and/or by at least 2-fold, e.g., enhancing the efficacy of a cell-based immunotherapy by 50%, 80%, 100%, 2-fold, 5-fold, 10-fold, 20-fold, 50-fold, 100-fold or more, as compared to the efficacy in the absence of the immune cells expressing the co-stimulatory polypeptides and/or the CAR polypeptide.

In any of the compositions or methods described herein, the immune cells (e.g., NK and/or T cells) may be autologous to the subject, i.e., the immune cells may be obtained from the subject in need of the treatment, genetically engineered for expression of co-stimulatory polypeptides and/or the CAR polypeptides, and then administered to the same subject. In one specific embodiment, prior to re-introduction into the subject, the autologous immune cells (e.g., T lymphocytes or NK cells) are activated and/or expanded ex vivo. Administration of autologous cells to a subject may result in reduced rejection of the host cells as compared to administration of non-autologous cells.

Alternatively, the host cells are allogeneic cells, i.e., the cells are obtained from a first subject, genetically engineered for expression of the co-stimulatory polypeptide and/or the CAR polypeptide, and administered to a second subject that is different from the first subject but of the same species. For example, allogeneic immune cells may be derived from a human donor and administered to a human recipient who is different from the donor. In a specific embodiment, the T lymphocytes are allogeneic T lymphocytes in which the expression of the endogenous T cell receptor has been inhibited or eliminated. In one specific embodiment, prior to introduction into the subject, the allogeneic T lymphocytes are activated and/or expanded ex vivo. T lymphocytes can be activated by any method known in the art, e.g., in the presence of anti-CD3/CD28, IL-2, phytohemoagglutinin, engineered artificial stimulatory cells or particles, or a combination thereof.

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-18, IL-21 protein, and K562 cell line, and/or engineered artificial stimulatory cells or particles. See, e.g., U.S. Pat. Nos. 7,435,596 and 8,026,097 for the description of useful methods for expanding NK cells. For example, NK cells used in the compositions or methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45(3): 321-334 (1975); Klein et al., Int. J. Cancer 18: 421-431 (1976)], and the Wilms tumor cell line HFWT (Fehniger et al., Int Rev Immunol 20(3-4):503-534 (2001); Harada H, et al., Exp Hematol 32(7):614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93: 313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the described compositions or methods, introduction (or re-introduction) of T lymphocytes, NK cells, or T lymphocytes and NK cells to the subject is followed by administering to the subject a therapeutically effective amount of IL-2.

In accordance with the present disclosure, patients can be treated by infusing therapeutically effective doses of immune cells such as T lymphocytes or NK cells comprising a co-stimulatory polypeptide and/or a CAR polypeptide of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/Kg will be infused, escalating to 10⁸ or more cells/Kg. IL-2 can be co-administered to expand infused cells. The amount of IL-2 can about 1-5×10⁶ international units per square meter of body surface.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The efficacy of the compositions or methods described herein may be assessed by any method known in the art and would be evident to a skilled medical professional. For example, the efficacy of the compositions or methods described herein may be assessed by survival of the subject or cancer or pathogen burden in the subject or tissue or sample thereof. In some embodiments, the compositions and methods described herein may be assessed based on the safety or toxicity of the therapy (e.g., administration of the immune cells expressing the co-stimulatory polypeptides and the CAR polypeptides) in the subject, for example, by the overall health of the subject and/or the presence of adverse events or severe adverse events.

### B. Combination Treatments

The compositions and methods described in the present disclosure may be utilized in conjunction with other types of therapy for cancer, such as chemotherapy, surgery, radiation, gene therapy, and so forth, or anti-infection therapy. Such therapies can be administered simultaneously or sequentially (in any order) with the immunotherapy according to the present disclosure. When co-administered with an additional therapeutic agent, suitable therapeutically effective dosages for each agent may be lowered due to the additive action or synergy.

In some instances, the immune cells (e.g., T lymphocytes and/or NK cells) expressing any of the co-stimulatory polypeptides and/or the anti-GPC3 CAR polypeptides disclosed herein may be administered to a subject who has been treated or is being treated with an additional therapeutic agent (e.g., an additional anti-cancer therapeutic agent). For example, the immune cells may be administered to a human subject simultaneously with the additional therapeutic agent. Alternatively, the immune cells may be administered to a human subject before the additional therapeutic agent. Alternatively, the immune cells may be administered to a human subject after the additional therapeutic agent.

Genetically engineered immune cells (e.g., T cells or NK cells) that co-express a co-stimulatory polypeptide and a CAR polypeptide specific to a tag can be co-used with a therapeutic agent conjugated to the tag. Via the therapeutic agent, which is capable of binding to an antigen associated with diseased cells such as tumor cells, such genetically engineered immune cells can be engaged with the diseased cells and inhibit their growth.

The treatments of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAGS, TIM3, etc.), therapeutic antibodies (e.g., for ADCC or ADC), or activators (including but not limited to agents that enhance 41BB, OX40, etc.).

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamine oxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (brefeldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); AKT inhibitors (such as MK-2206 2HC1, Perifosine (KRX-0401), GSK690693, Ipatasertib (GDC-0068), AZD5363, uprosertib, afuresertib, or triciribine); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

For examples of additional useful agents see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

The administration of an additional therapeutic agent can be performed by any suitable route, including systemic administration as well as administration directly to the site of the disease (e.g., to a tumor).

In some embodiments, the method involves administering the additional therapeutic agent to the subject in one dose. In some embodiments, the method involves administering the additional therapeutic agent to the subject in multiple doses (e.g., at least 2, 3, 4, 5, 6, 7, or 8 doses). In some embodiments, the additional therapeutic agent is administered to the subject in multiple doses, with the first dose of the additional therapeutic agent administered to the subject about 1, 2, 3, 4, 5, 6, or 7 days prior to administration of the immune cells expressing the co-stimulatory polypeptide and/or the CAR polypeptide. In some embodiments, the first dose of the additional therapeutic agent is administered to the subject between about 24-48 hours prior to the administration of the immune cells expressing the co-stimulatory polypeptide and/or the CAR polypeptide.

In some embodiments, the additional therapeutic agent is administered to the subject prior to administration of the immune cells expressing the co-stimulatory polypeptide and/or the CAR polypeptide and then subsequently about every two weeks. In some embodiments, the first two doses of the additional therapeutic agent are administered about one week (e.g., about 6, 7, 8, or 9 days) apart. In certain embodiments, the third and following doses are administered about every two weeks.

In any of the embodiments described herein, the timing of the administration of the additional therapeutic agent is approximate and includes three days prior to and three days following the indicated day (e.g., administration every three weeks encompasses administration on day 18, day 19, day 20, day 21, day 22, day 23, or day 24).

The efficacy of the methods described herein may be assessed by any method known in the art and would be evident to a skilled medical professional and/or those described herein. For example, the efficacy of the cell-based immunotherapy may be assessed by survival of the subject or cancer burden in the subject or tissue or sample thereof. In some embodiments, the cell-based immunotherapy is assessed based on the safety or toxicity of the therapy (e.g., administration of the the immune cells expressing the co-stimulatory polypeptides and/or the CAR polypeptides) in the subject, for example by the overall health of the subject and/or the presence of adverse events or severe adverse events.

### V. Kits for Therapeutic Use

The present disclosure also provides kits for use of the compositions described herein. For example, the present disclosure also provides kits comprising a population of immune cells (e.g., T lymphocytes or NK cells) that express co-express a co-stimulatory polypeptide and an anti-GPC3 CAR polypeptide for use in inhibiting the growth of diseased cells, e.g., tumor cells and/or enhancing immune cell growth and/or proliferation in a low glucose environment, a low amino acid environment, a low-pH environment, and/or hypoxic environment, for example, in a tumor microenvironment. The kit may further comprise a therapeutic agent conjugated to a tag (e.g., those described herein), to which the CAR polypeptide expressed on the immune cells bind. Such kits may include one or more containers comprising the population of the genetically engineered immune cells as described herein (e.g., T lymphocytes and/or NK cells), which co-express a co-stimulatory polypeptides and a CAR polypeptide such as those described herein, and optionally a therapeutic agent conjugated to a tag.

In some embodiments, the kit described herein comprises co-stimulatory polypeptide-expressing and CAR-expressing immune cells, which are expanded in vitro, and an antibody specific to a cell surface antibody that is present on activated T cells, for example, an anti-CD5 antibody, an anti-CD38 antibody or an anti-CD7 antibody. The co-stimulatory polypeptide-expressing and CAR-expressing immune cells may express any of the CAR constructs known in the art or disclosed herein.

Alternatively, the kit disclosed herein may comprise a nucleic acid or a nucleic acid set as described herein, which collectively encodes any of the CAR polypeptides and any of the co-stimulatory polypeptides as also described herein.

In some embodiments, the kit can additionally comprise instructions for use in any of the methods described herein. The included instructions may comprise a description of administration of the first and second pharmaceutical compositions to a subject to achieve the intended activity, e.g., inhibiting target cell growth in a subject, and/or enhancing the growth and/or proliferation of immune cells in a low-glucose environment, a low amino acid (e.g., a low glutamine environment) environment, a low pH environment, and/or a hypoxic environment (e.g., a low glucose, low amino acid, low pH or hyposic tumor microenvironment). The kit may further comprise a description of selecting a subject suitable for treatment based on identifying whether the subject is in need of the treatment. In some embodiments, the instructions comprise a description of administering the population of genetically engineered immune cells and optionally a description of administering the tag-conjugated therapeutic agent.

The instructions relating to the use of the immune cells and optionally the tag-conjugated therapeutic agent as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the first pharmaceutical composition is a population of immune cells (e.g., T lymphocytes or NK cells) that express a CAR polypeptide and a co-stimulatory polypeptide as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R.I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: The Activity of T Cells Expressing Anti-GPC3 CAR Variants is Enhanced by Co-Expressing Costimulatory Polypeptides

This example demonstrates that expressing tumor necrosis factor (TNF) superfamily costimulatory polypeptides or B7/CD28 superfamily costimulatory peptides in T cells in combination with an anti-GPC3 CAR can enhance the activity of the T cell relative to the anti-GPC3 CAR alone.

In these experiments, T cells were transduced with virus encoding an anti-GPC3 CAR polypeptide with a 4-1BB costimulatory domain (GPC3-CAR-4-1BB; SEQ ID NO: 1) alone, an anti-GPC3 CAR polypeptide with a CD28 costimulatory domain (GPC3-CAR-CD28; SEQ ID NO: 1) alone, or each of these CAR variants in combination with costimulatory polypeptides CD30L, CD40L, CD70, GITRL, ICOSL, LIGHT, OX40L, TL1A, BAFFR, CD40, CD27, OX40, ICOS, and 4-1BB. Transduced T cells were evaluated in a panel of functional assays including proliferation, cytokine release, cytotoxicity, and repeated stimulation (see assay details in below examples). Results obtained from this study showed that the combination of either anti-GPC3 CAR or both with one or more of the above-listed co-stimulatory polypeptides enhanced T cell proliferation, increased production of certain cytokines, and/or enhanced cytotoxicity.

These experiments demonstrate that expressing tumor necrosis factor (TNF) superfamily costimulatory polypeptides or B7/CD28 superfamily costimulatory peptides in T cells in combination with an anti-GPC3 CAR can enhance the activity of the T cell relative to the anti-GPC3 CAR alone in the context of both anti-GPC3 CAR polypeptide with a 4-1BB costimulatory domain and an anti-GPC3 CAR polypeptide with a CD28 costimulatory domain. The costimulatory polypeptides that impart improved activity vary depending on which CAR variant is co-expressed in the same T cell.

### Example 2: The Enhanced Activity of T Cells Expressing Anti-GPC3 CAR and TNF Costimulatory Polypeptides is Dependent on the Identity of the Costimulatory Domain in the CAR in Repeated Stimulation Assays

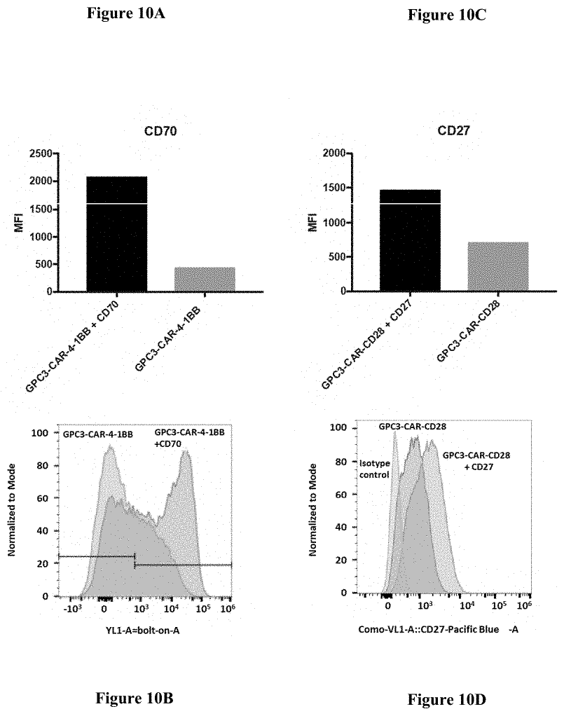

This example demonstrates that expressing tumor necrosis factor (TNF) superfamily costimulatory polypeptides CD70, LIGHT, and OX40L in T cells in combination with an anti-GPC3 CAR enhances the activity of the T cell relative to the anti-GPC3 CAR alone in the presence of target cells under multiple restimulation conditions and that the level of enhancement is dependent on the identity of the costimulatory domain in the CAR. In these experiments, T cells were transduced with virus encoding an anti-GPC3 CAR polypeptide with a 4-1BB costimulatory domain (GPC3-CAR-4-1BB; SEQ ID NO: 1) alone, an anti-GPC3 CAR polypeptide with a CD28 costimulatory domain (GPC3-CAR-CD28; SEQ ID NO: 2) alone, or each of these CAR variants and CD70 (SEQ ID NO: 34), LIGHT (SEQ ID NO: 43), or OX40L (SEQ ID NO: 47) separated by a P2A ribosomal skip sequence. T cells expressing GPC3-CAR-4-1BB and cells co-expressing GPC3-CAR-4-1BB and CD70 were evaluated for CD70 expression by flow cytometry by staining with an anti-CD70 antibody. T cells co-expressing GPC3-CAR-4-1BB and CD70 showed more CD70 surface expression, as evidenced by a higher mean fluorescence intensity, than T cells expressing GPC3-CAR-4-1BB alone (FIGS. 10A and 10B).

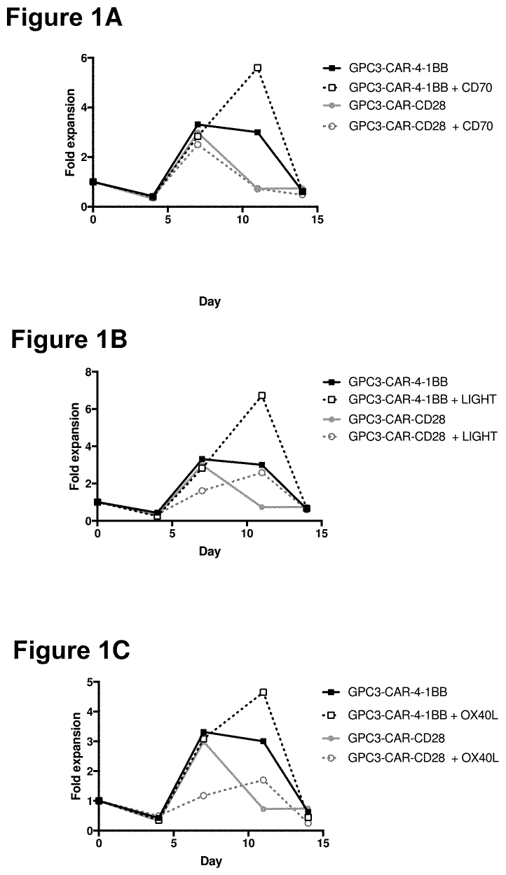

Transduced T cells (effector) and GPC3-expressing Hep3B cells (target) were incubated at a 2:1 effector-to-target ratio (100,000 effector cells; 50,000 target cells) in a 200-μL reaction volume in RPMI 1640 media supplemented with 10% fetal bovine serum. Reactions were incubated at 37° C. in 5% CO₂ incubator. Every 3 or 4 days, T cells were restimulated by transferring half the volume of T cells to new plates containing 50,000 fresh target cells (in 100 μL media), and the final volume was adjusted to 200 μL. Cells were restimulated 3 times. At each time point, the remaining cells were stained with an anti-CD3 antibody and a live/dead stain. The number live, CD3-positive cells were evaluated by flow cytometry as a measure of T cell proliferation. The fold T cell expansion relative to the previous time point was plotted as a function of time (FIGS. 1A-1C).

T cells co-expressing GPC3 CAR-4-1BB and CD70 showed similar or superior expansion relative to T cells expressing GPC3 CAR-4-1BB alone after all stimulation rounds (FIG. 1A). In contrast, T cells co-expressing GPC3 CAR-CD28 and CD70 showed similar expansion relative to T cells expressing GPC3 CAR-CD28 alone after all stimulation rounds. T cells co-expressing GPC3 CAR-4-1BB and LIGHT showed similar or superior expansion relative to T cells expressing GPC3 CAR-4-1BB alone after all stimulation rounds (FIG. 1B). In contrast, T cells co-expressing GPC3 CAR-CD28 and LIGHT showed similar expansion relative to T cells expressing GPC3 CAR-CD28 alone at most time points and a modest improvement in expansion after the third round of simulation. T cells co-expressing GPC3 CAR-4-1BB and OX40L showed similar or superior expansion relative to T cells expressing GPC3 CAR-4-1BB alone after all stimulation rounds (FIG. 1C). In contrast, T cells co-expressing GPC3 CAR-CD28 and OX40L showed similar or inferior expansion relative to T cells expressing GPC3 CAR-CD28 alone at most time points and a modest improvement in expansion after the third round of simulation.

These experiments demonstrate that co-expressing TNF superfamily member polypeptides like CD70, LIGHT, and OX40L in T cells that also express anti-GPC3 CAR with a 4-1BB costimulatory domain can enhance T cell activity after multiple restimulations. In contrast, CD70, LIGHT, and OX40L do not enhance the activity of T cells when co-expressed with an anti-GPC3-CD28 CAR.

### Example 3: T Cells Co-Expressing Anti-GPC3 CAR with a 4-1BB Costimulatory Domain and TNF Superfamily Member Polypeptides CD70, LIGHT, and OX40L Show Enhanced Proliferation and Cytokine Release in a Repeated Stimulation Assay

This example demonstrates that expressing tumor necrosis factor (TNF) superfamily costimulatory polypeptides CD70, LIGHT, and OX40L in T cells in combination with an anti-GPC3 CAR with a 4-1BB costimulatory domain enhances the activity of the T cell relative to the anti-GPC3 CAR alone. In these experiments, T cells were transduced with virus encoding an anti-GPC3 CAR polypeptide with a 4-1BB costimulatory domain (GPC3-CAR-4-1BB; SEQ ID NO: 1) or virus encoding GPC3-CAR-4-1BB and CD70 (SEQ ID NO: 34), LIGHT (SEQ ID NO: 43), or OX40L (SEQ ID NO: 47) separated by a P2A ribosomal skip sequence.

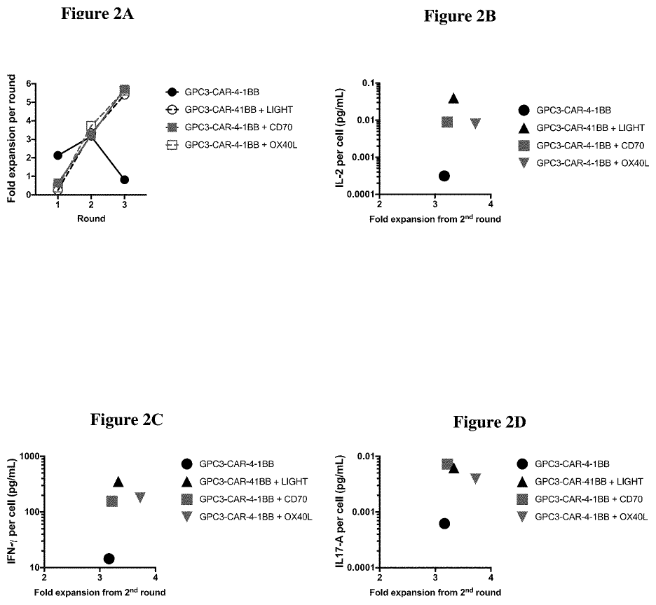

Transduced T cells (effector) and GPC3-expressing JHH7 cells (target) were incubated at a 2:1 effector-to-target ratio (100,000 effector cells; 50,000 target cells) in a 200-μL reaction volume in RPMI 1640 media supplemented with 10% fetal bovine serum. Reactions were incubated at 37° C. in 5% CO₂ incubator. At day 3 and 6, T cells were restimulated by transferring half the volume of T cells to new plates containing 50,000 fresh target cells (in 100 μL media), and the final volume was adjusted to 200 μL. At each time point, the remaining cells were stained with an anti-CD3 antibody and a live/dead stain. The number live, CD3-positive cells were evaluated by flow cytometry as a measure of T cell proliferation. The fold T cell expansion relative to the previous time point was plotted as a function of time (FIG. 2A) at each restimulation round time point.

Supernatants were removed from the reactions on day 4, 24 hr after the second stimulation and analyzed for cytokine production. Cytokines were measured using a U-PLEX assay kit (Meso Scale Discovery) according to the manufacturer's instructions. IL-2, IFN-gamma, and IL-17A measurements were normalized based on the number of cells in the well measured on day 3 to give a pg/mL/cell value and plotted as a function of fold-expansion observed after the day 3 stimulation, as measured on day 6 measurement (FIGS. 2B-2C).

T cells co-expressing GPC3 CAR-4-1BB and CD70, LIGHT, or OX40L showed similar expansion relative to T cells expressing GPC3 CAR-4-1BB alone after the first two stimulation rounds and superior expansion after the third round of stimulation (FIG. 2A). T cells co-expressing GPC3 CAR-4-1BB and CD70, LIGHT, or OX40L showed superior IL-2 (FIG. 2B), IFN-gamma (FIG. 2C), and IL17-A (FIG. 2D) relative to T cells expressing GPC3 CAR-4-1BB alone 24 hr after the second stimulation round.

These experiments demonstrate that co-expressing TNF superfamily member polypeptides like CD70, LIGHT, and OX40L in T cells that also express anti-GPC3 CAR with a 4-1BB costimulatory domain can enhance T cell activity.

### Example 4: T Cells Co-Expressing Anti-GPC3 CAR with a 4-1BB Costimulatory Domain and TNF Superfamily Member Polypeptides CD70, LIGHT, and OX40L Show Enhanced Cytokine Release and Proliferation

This example demonstrates that expressing tumor necrosis factor (TNF) superfamily costimulatory polypeptides CD70, LIGHT, and OX40L in T cells in combination with an anti-GPC3 CAR with a 4-1BB costimulatory domain enhances the activity of the T cell relative to the anti-GPC3 CAR alone. In these experiments, T cells were transduced with virus encoding an anti-GPC3 CAR polypeptide with a 4-1BB costimulatory domain (GPC3-CAR-4-1BB; SEQ ID NO: 1) or virus encoding GPC3-CAR-4-1BB and CD70 (SEQ ID NO: 34), LIGHT (SEQ ID NO: 43), or OX40L (SEQ ID NO: 47) separated by a P2A ribosomal skip sequence.

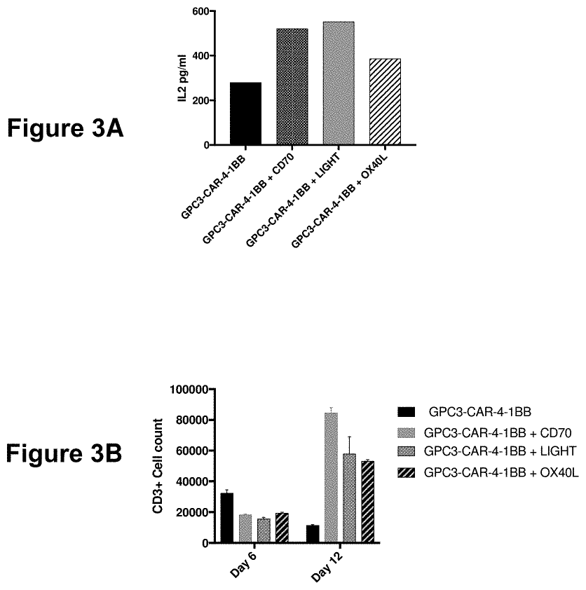

Transduced T cells (effector) and GPC3-expressing Hep3B cells (target) were plated at a 2:1 effector-to-target ratio (100,000 effector cells; 50,000 target cells) and incubated at 37° C. in a 5% CO₂ incubator for 24 hr. Supernatant was removed from the reaction and analyzed for IL-2 using a Human IL-2 Assay Kit (Cisbio) according to the manufacturer's instructions. The concentration of IL-2 in the supernatant was plotted as a function of variant tested (FIG. 3A). T cells co-expressing GPC3-CAR-4-1BB and CD70, LIGHT, or OX40L all demonstrated superior IL-2 production relative to T cells expressing GPC3-CAR-4-1BB alone.

Transduced T cells (effector) and GPC3-expressing HepG2 cells (target) were mixed at a 1:1 effector-to-target ratio and incubated at 37° C. in a 5% CO₂ incubator for 12 days. Samples were taken at day 6 and day 12 and stained with a viability dye and an anti-CD3 antibody and analyzed by flow cytometry. The number of live CD3+ cells, which is a measure of T cell proliferation, was plotted as a function of variant tested and time point (FIG. 3B). T cells co-expressing GPC3-CAR-4-1BB and CD70, LIGHT, or OX40L showed a similar level of proliferation at day 6 and superior proliferation at day 12 relative to T cells expressing the GPC3-CAR-4-1BB alone.

These experiments demonstrate that co-expressing TNF superfamily member polypeptides like CD70, LIGHT, and OX40L in T cells that also express anti-GPC3 CAR with a 4-1BB costimulatory domain can enhance T cell activity.

### Example 5: T Cells Co-Expressing Anti-GPC3 CAR with a 4-1BB Costimulatory Domain and CD70 Show Superior Activity Relative to T Cells Co-Expressing Anti-GPC3 CAR with a 4-1BB Costimulatory Domain and LIGHT or OX40L

This example demonstrates that, relative to other tumor necrosis factor (TNF) superfamily members, the costimulatory polypeptide CD70 (SEQ ID NO: 34) provides substantial functional advantage when combined with an anti-GPC3 CAR containing a 4-1BB primary costimulatory domain (GPC3-CAR-4-1BB; SEQ ID NO: 1). In these experiments, T cells were transduced with virus encoding a CAR polypeptide (SEQ ID NO: 1) alone or a CAR polypeptide and CD70 (SEQ ID NO: 34), LIGHT (SEQ ID NO: 43), or OX40L (SEQ ID NO: 47) separated by a P2A ribosomal skip sequence.

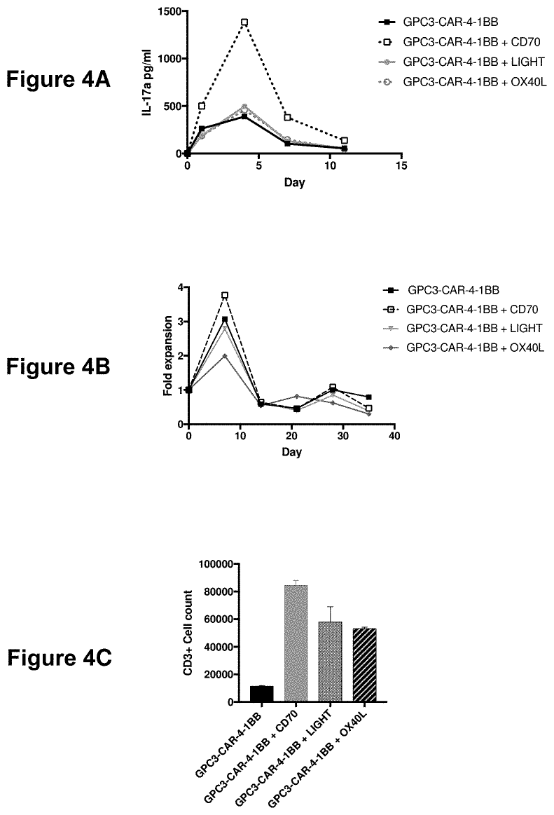

For some experiments, transduced T cells T (effector) and Hep3B cells (target) were plated at a 2:1 effector-to-target ratio (100,000 effector cells; 50,000 target cells) in RPMI 1640 media supplemented with 10% heat inactivated fetal bovine serum (PBS), with re-stimulation every 3 to 4 days with 50,000 fresh target cells. Cytokine production (IL-17A) was measured from the culture supernatants over time using a U-PLEX assay kit (Meso Scale Discovery) according to the manufacturer's instructions. IL-17A levels are depicted throughout the course of the experiment as pg/mL (FIG. 4A). T cells co-expressing GPC3-CAR-4-1BB and CD70 showed superior IL-17A production relative to T cells expressing GPC3-CAR-4-1BB alone and to T cells co-expressing GPC3-CAR-4-1BB and LIGHT or OX40L.

In other experiments, transduced T cells T (effector) and Hep3B cells (target) were plated at a 2:1 effector-to-target ratio (100,000 effector cells; 50,000 target cells) in RPMI 1640 media supplemented with 10% heat inactivated fetal bovine serum (FBS), with re-stimulation every 7 days with 50,000 fresh target cells; the number CD3-positive cells were evaluated by flow cytometry as a measure of T cell proliferation over time. Proliferation of CAR T cells is represented as a fold change relative to the previous time point (FIG. 4B). T cells co-expressing GPC3-CAR-4-1BB and CD70 showed superior proliferation relative to T cells expressing GPC3-CAR-4-1BB alone and to T cells co-expressing GPC3-CAR-4-1BB and LIGHT or OX40L.

In other experiments, transduced T cells (effector) were plated with HepG2 target cells at a 1:1 effector-to-target ratio (30,000 effector cells; 30,000 target cells) in RPMI 1640 media supplemented with 10% heat inactivated fetal bovine serum (FBS), and the number CD3-positive cells were evaluated by flow cytometry as a measure of T cell proliferation after 12 days. Proliferation of T cells is represented as to the total CD3+ T cell count (FIG. 4C). T cells co-expressing GPC3-CAR-4-1BB and CD70 showed superior proliferation relative to T cells expressing GPC3-CAR-4-1BB alone and to T cells co-expressing GPC3-CAR-4-1BB and LIGHT or OX40L.

Together experiments demonstrate that, relative to other tumor necrosis factor (TNF) superfamily members, the costimulatory polypeptide CD70 (SEQ ID NO: 34) provides substantial functional advantage when combined with an anti-GPC3 CAR containing a 4-1BB primary costimulatory domain (SEQ ID NO: 1).

### Example 6: T Cells Co-Expressing Anti-GPC3 CAR with a CD28 Costimulatory Domain and TNF Superfamily Member Polypeptide CD27 Show Enhanced Cytokine Release and Proliferation

This example demonstrates that the costimulatory polypeptide CD27 (SEQ ID NO: 33) provides substantial functional advantage to T cells when combined with an anti-GPC3 CAR containing a CD28 primary costimulatory domain (GPC3-CAR-CD28; SEQ ID NO: 2). In these experiments, T cells were transduced with virus encoding a CAR polypeptide (SEQ ID NO: 2) alone or a CAR polypeptide and CD27 (SEQ ID NO: 33), separated by a P2A ribosomal skip sequence. T cells expressing GPC3-CAR-CD28 and cells co-expressing GPC3-CAR-CD28 and CD27 were evaluated for CD27 expression by flow cytometry by staining with an anti-CD27 antibody. T cells co-expressing GPC3-CAR-CD28 and CD27 showed more CD27 surface expression, as evidenced by a higher mean fluorescence intensity, than T cells expressing GPC3-CAR-CD28 alone (FIGS. 10C-10D).

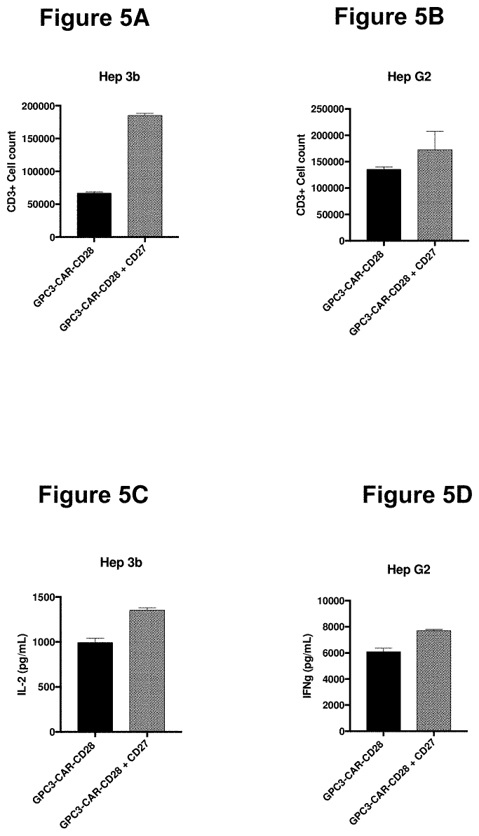

In some experiments, T cells and Hep3B were mixed at an E:T ratio of 2:1 (60,000 effector cells; 30,000 target cells) in RPMI 1640 media supplemented with 10% heat inactivated fetal bovine serum (FBS), followed by incubation for 7 days. T cell proliferation was measured by flow cytometry. The number CD3-positive cells were plotted as a function of T cell variant (FIG. 5A). Similarly, T cell proliferation after single stimulation with HepG2 target cells at a 1:1 effector-to-target ratio (30,000 effector cells; 30,000 target cells) in RPMI 1640 media supplemented with 10% heat inactivated fetal bovine serum (FBS) was also evaluated (FIG. 5B). These experiments demonstrate that a T cells expressing GPC3-CAR-4-1BB and CD27 sequence has improved proliferation relative to T cells expressing GPC3-CAR-4-1BB alone.

In some experiments, T cells (effector) and Hep3B or HepG2 cells (target) were plated at a 4:1 effector-to-target ratio (120,000 effector cells; 30,000 target cells) in RPMI 1640 media supplemented with 10% heat inactivated fetal bovine serum (FBS), followed by 24 hrs incubation. IL-2 (Hep3B, FIG. 5C) and IFN-gamma (HepG2, FIG. 5D) were measured from reaction supernatants using Human IL-2 Assay Kit (Cisbio) or a Human IFN-gamma Assay Kit assay (Cisbio), respectively, according to the manufacturer's instructions. These experiments demonstrate that a T cells expressing GPC3-CAR-4-1BB and CD27 sequence has improved cytokine production relative to T cells expressing GPC3-CAR-4-1BB alone.

This example demonstrates that the costimulatory polypeptide CD27 provides substantial functional advantage to T cells when combined with an anti-GPC3 CAR containing a CD28 primary costimulatory domain.

### Example 7: T Cells Co-Expressing Anti-GPC3 CAR with a CD28 Costimulatory Domain and TNF Superfamily Member Polypeptide CD27 Show Enhanced Activity in the Presence of Suppressive MDSCs and Regulatory T Cells

This example demonstrates that the costimulatory polypeptide CD27 (SEQ ID NO: 33) provides substantial functional advantage to T cells when combined with an anti-GPC3 CAR containing a CD28 primary costimulatory domain (GPC3-CAR-CD28; SEQ ID NO: 2) in assays containing suppressive myeloid-derived suppressor cells (MDSCs) or regulatory T cells (Treg). In these experiments, T cells were transduced with virus encoding GPC3-CAR-CD28 (SEQ ID NO: 2) alone or a GPC3-CAR-CD28 and CD27 (SEQ ID NO: 33), separated by a P2A ribosomal skip sequence.

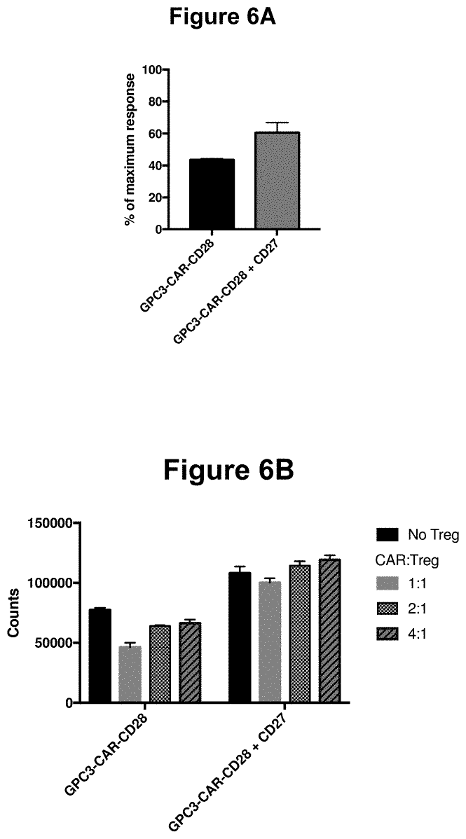

In some experiments, MDSCs were generated from CD14+ monocytes from donor matched PBMCs. Briefly, CD14-positive cells were isolated using the EasySep Human CD14 Positive Selection Kit II (Gibco) according to the manufacturer's protocol. CD14+ cells were cultured in RPMI 1640 media supplemented with 10% fetal bovine serum in the presence of GMCSF (10 ng/mL) and PGE₂ (1 ng/mL). Cells were incubated in a CO₂ (5%) incubator at 37 degrees C. for 6 days. Cultures were supplemented with GMCSF (10 ng/mL) and PGE₂ (1 ng/mL) on day 2; on day 4, media was removed and replenished with fresh RPMI 1640 supplemented with 10% fetal bovine serum and GMCSF (10 ng/mL) and PGE₂ (1 ng/mL). Cells were harvested for use in assays as MDSCs at day 6. Cells were characterized by flow cytometry to confirm that they were CD14_{low}/HLA-DR_{low}/CD33_{high}/PDL1_{high}. T cells (effector) and Hep G2 cells (target) were plated at a 2:1 effector-to-target ratio (100,000 effector cells; 50,000 target cells) in the presence of 3:1 effector-to-MDSCs and incubated for 7 days at 37° C.+5% CO₂. Culture media contained recombinant annexin V protein (1 μg/mL) to block phagocytosis of activated T cells by MDSCs. The number of live CAR+ CD3+ cells were evaluated by flow cytometry and results were expressed as percent of maximum response without MDSCs (FIG. 6A). T cells co-expressing GPC3-CAR-CD28 and CD27 showed a higher response than T cells expressing GPC3-CAR-CD28 alone, demonstrating a greater ability to overcome MDSC suppression.

In some experiments, inducible Tregs were generated from donor-matched PBMCs with rapamycin and hTGF-b and isolated using the Miltenyi CD4⁺/CD25⁺/CD127^{dim/-} Human Regulatory T Cell Isolation Kit II. T cells (effector) and Hep3b cells (target) were plated at a 2:1 effector-to-target ratio (100,000 effector cells; 50,000 target cells) in the presence of Tregs at varying ratios relative to Cell Trace Violet-labeled CAR-T cells (1:1, 1:2, 1:4 Treg to CAR-T cells) and incubated for 7 days at 37° C.+5% CO₂. The numbers of Cell Trace Violet-labeled CAR⁺ cells were evaluated by flow cytometry as a measure of proliferation (FIG. 6B). T cells co-expressing GPC3-CAR-CD28 and CD27 showed more proliferation than T cells expressing GPC3-CAR-CD28 alone, demonstrating a greater ability to overcome Treg suppression.

Together, these experiments demonstrate that a T cells co-expressing GPC3-CAR-CD28 and CD27 show superior ability to overcome immunosuppression exerted by either MDSCs or Tregs relative to T cells expressing GPC3-CAR-CD28 alone.

### Example 8: T Cells Co-Expressing Anti-GPC3 CAR with a 4-1BB Costimulatory Domain and TNF Superfamily Member Polypeptides CD70, LIGHT, or OX40L Show Enhanced Activity in Tumor Xenograft Models in Mice

This example demonstrates that expression of tumor necrosis factor (TNF) superfamily costimulatory peptides CD70, LIGHT and OX40L in GPC3-CAR-4-1BB results in increased anti-tumor activity in GPC3-expressing xenograft models in mice, relative to GPC3-CAR-4-1BB alone. Subcutaneous human hepatocellular carcinoma (HCC) xenograft models (Hep G2, Hep 3b and JHH7) were established in NSG™ (NOD scid gamma, NOD.Cg-Prkdcscid IL2rgtm1Wjl/SzJ, Strain 005557) mice.

Hep G2 HCC (ATCC HB-8065) xenografts were established by subcutaneous injection with 5×10⁶ cells in the right flank. Treatment with GPC3 CAR-T cells was initiated when tumor volumes reached approximately 100 mm³ (day 19 post inoculation). Mice were randomized into treatment groups of 5 mice each, based on tumor volume, and treated with T cells expressing GPC3-CAR-4-1BB (SEQ ID NO: 1) alone or GPC3-CAR-4-1BB and CD70 (SEQ ID NO: 34), LIGHT (SEQ ID NO: 43) or OX40L (SEQ ID NO: 47) at a dose of 5×10⁵ CAR+ cells intravenously on days 1 and 8. Tumor volume and body weights were measured two-to-three times weekly for the duration of the experiment.

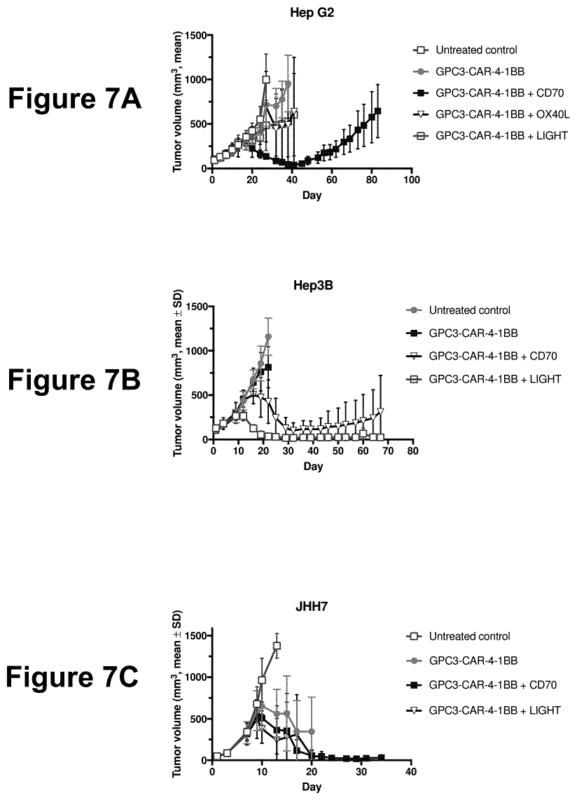

T cells expressing GPC3-CAR-4-1BB and T cells co-expressing GPC3-CAR-4-1BB and LIGHT were inactive against Hep G2 xenografts at the CAR dose evaluated; tumor growth was comparable to untreated controls (FIG. 7A). T cells co-expressing GPC3-CAR-4-1BB and OX40L were moderately more active than T cells expressing GPC3-CAR-4-1BB alone, with a heterogeneous response among the 5 animals. T cells co-expressing GPC3-CAR-4-1BB and CD70 were highly active, resulting in complete tumor regressions in all animals by day 40, with subsequent relapse in all animals.

Hep 3b HCC (ATCC, HB-8064) xenografts were established by subcutaneous injection with 5×10⁶ cells in the right flank. Treatment with GPC3 CAR-T cells was initiated when tumor volumes reached approximately 100 mm³ (day 20 post inoculation). Mice were randomized into treatment groups of 5 mice each, based on tumor volume, and treated with T cells expressing GPC3-CAR-4-1BB alone, T cells co-expressing GPC3-CAR-4-1BB and CD70, or T cells co-expressing GPC3-CAR-4-1BB and LIGHT at a dose of 1×10⁶ CAR+ cells intravenously on days 1 and 8. Tumor volume and body weights were measured two-to-three times weekly for the duration of the experiment.

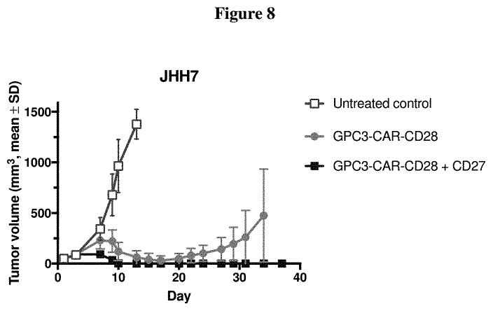

T cells expressing GPC3-CAR-4-1BB were inactive against Hep 3b xenografts at the CAR dose evaluated; tumor growth was comparable to untreated controls (FIG. 8B). T cells co-expressing GPC3-CAR-4-1BB and CD70 were highly active, with complete tumor regressions in 4 of 5 animals, with all tumors relapsing after day 60. T cells co-expressing GPC3-CAR-4-1BB and LIGHT were most active in the study, resulting in complete tumor regressions in all animals, with relapse in 2 of 5 animals after day 70.

JHH7 HCC (JCRB, 1031) xenografts were established by subcutaneous injection with 5×10⁶ cells in the right flank. Treatment with GPC3 CAR-T cells was initiated when tumor volumes reached approximately 50 mm³ (day 8 post inoculation). Mice were randomized into treatment groups of 5 mice each, based on tumor volume, and treated with T cells expressing GPC3-CAR-4-1BB alone, T cells co-expressing GPC3-CAR-4-1BB and CD70, or T cells co-expressing GPC3-CAR-4-1BB and LIGHT at a dose of 5×10⁶ CAR+ cells intravenously on days 1 and 8. Tumor volume and body weights were measured two-to-three times weekly for the duration of the experiment.

T cells expressing GPC3-CAR-4-1BB were moderately active against JHH7 xenografts at the CAR dose evaluated, with a heterogeneous response among the treatment group (FIG. 7C). In three animals, tumor growth was comparable to untreated controls, while 2 of 5 animals experienced complete tumor regressions. T cells co-expressing GPC3-CAR-4-1BB and LIGHT were highly active, with tumor regressions in 4 of 5 animals including two complete responses. T cells co-expressing GPC3-CAR-4-1BB and CD70 were highly active, with complete tumor regressions in 4 of 5 animals. There were no tumor relapses in any of the animals with complete regressions across GPC3-CAR-4-1BB treatment groups.

These experiments demonstrate that T cells co-expressing anti-GPC3 CAR with a 4-1BB costimulatory domain and TNF superfamily members CD70, LIGHT and OX40L show enhanced anti-tumor activity in xenograft models in mice relative to T cells expressing anti-GPC3 CAR with a 4-1BB costimulatory domain alone.

### Example 9: T Cells Co-Expressing Anti-GPC3 CAR with a CD28 Costimulatory Domain and TNF Superfamily Member Polypeptides CD27 Shows Enhanced Activity in Tumor Xenograft Models in Mice

This example demonstrates that co-expression of tumor necrosis factor (TNF) superfamily costimulatory peptides CD27 and GPC3-CAR-CD28 in T cells results in increased anti-tumor activity in GPC3-expressing xenograft models in mice, relative to T cells expressing GPC3-CAR-CD28 alone. Subcutaneous human hepatocellular carcinoma (HCC) xenograft models (JHH7) were established in NSG™ (NOD scid gamma, NOD.Cg-Prkdcscid IL2rgtm1Wjl/SzJ, Strain 005557) mice.

JHH7 HCC (JCRB, 1031) xenografts were established by subcutaneous injection with 5×10⁶ cells in the right flank. Treatment with GPC3 CAR-T cells was initiated when tumor volumes reached approximately 50 mm³ (day 8 post inoculation). Mice were randomized into treatment groups of 5 mice each, based on tumor volume, and treated with T cells expressing GPC3-CAR-CD28 (SEQ ID NO: 2) alone or T cells co-expressing GPC3-CAR-CD28 and CD27 (SEQ ID NO: 33) at a dose of 5×10⁶ CAR+ cells intravenously on days 1 and 8. Tumor volume and body weights were measured two-to-three times weekly for the duration of the experiment.

T cells expressing GPC3-CAR-CD28 alone were highly active against JHH7 xenografts at the CAR dose evaluated, resulting in complete tumor regressions in 4 of 5 animals by day 15 with subsequent relapse of all tumors (FIG. 8). T cells co-expressing GPC3-CAR-CD28 and CD27 were highly active, with tumor regressions in all animals by day 10 and continued tumor control throughout the remainder of the experiment with no tumor relapses.

These experiments demonstrate that T cells co-expressing anti-GPC3 CAR with a CD28 costimulatory domain and TNF superfamily members CD27 shows enhanced anti-tumor activity in xenograft models in mice relative to T cells expressing anti-GPC3 CAR with a CD28 costimulatory domain alone.

### Example 10: Expansion of T Cells Expressing Anti-GPC3 CAR Alone or in Combination with TNF Superfamily Polypeptides in Xenograft Models in Mice

This example demonstrates that expression of tumor necrosis factor (TNF) superfamily costimulatory peptides CD70 in GPC3-CAR-4-1BB and CD27 in GPC3-CAR-CD28 result in enhanced in vivo expansion of CAR-T in tumor-bearing NSG mice.

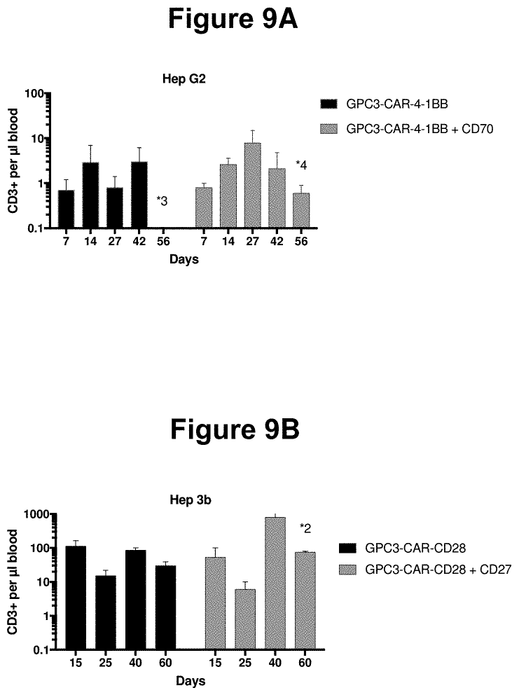

Animals bearing subcutaneous Hep G2 xenografts were treated with GPC3 CAR-T cells when tumor volumes reached approximately 100 mm³ (day 19 post inoculation). Groups of 5 mice each were treated with T cells expressing GPC3-CAR-4-1BB (SEQ ID NO: 1) or T cells co-expressing GPC3-CAR-4-1BB and CD70 (SEQ ID NO: 34) at a dose of 5×10⁵ CAR+ cells intravenously on days 1 and 8. Whole blood samples (20 μl) were collected by orbital bleed under isoflurane anesthesia on days 7, 14, 27, 42 and 56 and frozen with BamBanker cryoprotectant until processed for flow cytometry. Red blood cells were lysed, and samples were stained with live/dead stain and anti-human CD3 and analyzed by flow cytometry. Results are expressed as number of live CD3+ cells per μL of blood (FIG. 9A). Each time point represents the mean of 5 animals, with exceptions indicated with asterisks followed by the number of samples evaluated.

Human CD3+ cells were detected in the peripheral blood samples at all time points, with counts ranging from approximately 1 per μL on day 7 (prior to CAR dose 2) and with increased counts over time for both T cells expressing GPC3-CAR-4-1BB and T cells co-expressing GPC3-CAR-4-1BB and CD70 (FIG. 9A). Increased CD3+ cell counts were detected for T cells co-expressing GPC3-CAR-4-1BB and CD70, with counts peaking at day 27, and sustained persistence of T cell counts measured on day 56, whereas no CD3+ cells were detected at day 56 with T cells expressing GPC3-CAR-4-1BB alone. Expression of CD70 provides a benefit to GPC3-CAR-4-1BB CAR-T expansion and persistence in vivo. Animals bearing subcutaneous Hep 3b xenografts were treated with GPC3 CAR-T cells when tumor volumes reached approximately 100 mm³ (day 20 post inoculation). Groups of 5 mice each were treated with T cells expressing GPC3-CAR-CD28 (SEQ ID NO: 2) or T cells co-expressing GPC3-CAR-CD28 and CD27 (SEQ ID NO: 33) at a dose of 1×10⁶ CAR+ cells intravenously on days 1 and 8. Whole blood samples (20 μL) were collected by orbital bleed under isoflurane anesthesia on days 15, 25, 40 and 60 and frozen with BamBanker cryoprotectant until processed for flow cytometry. Red blood cells were lysed, and samples were stained with live/dead stain and anti-human CD3 and analyzed by flow cytometry. Results are expressed as number of live CD3+ cells per μl of blood in FIG. 9B. Each time point represents the mean of 5 animals, with exceptions indicated with asterisks followed by the number of samples evaluated.

Human CD3+ cells were detected in the peripheral blood samples at all time points, with counts ranging from approximately 100 per μL on day 15 and with fluctuating counts over time for both T cells expressing GPC3-CAR-CD28 alone and T cells co-expressing GPC3-CAR-CD28 and CD27. Increased CD3+ cell counts were detected for T cells co-expressing GPC3-CAR-CD28 and CD27 on days 40 and 60, with counts peaking at day 40, approximately 10-fold greater levels than with T cells expressing GPC3-CAR-CD28 alone. Expression of CD27 provides a benefit to GPC3-CAR-CD28 CAR-T expansion and persistence in vivo.

These experiments demonstrate that T cells co-expressing anti-GPC3 CAR variants and TNF superfamily polypeptides like CD70 and CD27 can show enhanced T cell expansion and persistence in vivo in tumor models in mice relative to T cells expressing anti-GPC3 CAR variants alone.

### Example 11: The In Vitro and In Vivo Activity of T Cells Expressing Anti-GPC3 CAR Variants is Enhanced by Co-Expressing Costimulatory Polypeptides

The above examples demonstrate that expressing tumor necrosis factor (TNF) superfamily costimulatory polypeptides or B7/CD28 superfamily costimulatory peptides in T cells in combination with an anti-GPC3 CAR can enhance the in vitro and in vivo activity of the T cell relative to the anti-GPC3 CAR alone. The above data can be summarized as follows:
GPC3-4-1BB CAR+CD70, GPC3-4-1BB CAR+LIGHT, and GPC3-4-1BB CAR+OX40L
   Improved proliferation relative to CAR-4-1BB parent in repeated stimulation assay relative to CAR-4-1BB parent
      Specific to CAR-4-1BB+LIGHT combination, CAR-CD28+LIGHT does not show improvement over CAR-CD28 parent
   Improved IL-2, IFN-gamma, and IL-17A production after repeated stimulation relative to CAR-4-1BB parent
   Improved proliferation relative to CAR-4-1BB parent in single stimulation proliferation assay
   Improved IL-2 production after stimulation relative to CAR-4-1BB parent
GPC3-4-1BB CAR+CD70, GPC3-4-1BB CAR+LIGHT
   Improved efficacy in tumor models in vivo in mice relative to CAR-4-1BB parent
GPC3-4-1BB CAR+CD70
   Increased T cell persistence in tumor models in vivo in mice relative to CAR-4-1BB parent
GPC3-CD28 CAR+CD27
   Improved proliferation relative to CAR-CD28 parent
   Improved IL-2 production relative to CAR-CD28 parent
   Improved resistance to MDSC suppression relative to CAR-CD28 parent
   Improved resistance to Treg suppression relative to CAR-CD28 parent
   Improved efficacy in tumor models in vivo in mice relative to CAR-CD28 parent

### Example 12: The Activity of T Cells Expressing Anti-GPC3 CAR Variants is Enhanced by Co-Expressing Costimulatory Polypeptides

This example demonstrates that expressing tumor necrosis factor (TNF) superfamily costimulatory polypeptides or B7/CD28 superfamily costimulatory peptides in T cells in combination with an anti-GPC3 CAR can enhance the activity of the T cell relative to the anti-GPC3 CAR alone.

In these experiments, T cells were transduced with virus encoding an anti-GPC3 CAR polypeptide with a 4-1BB costimulatory domain (GPC3-CAR-4-1BB; SEQ ID NO: 1) alone, an anti-GPC3 CAR polypeptide with a CD28 costimulatory domain (GPC3-CAR-CD28; SEQ ID NO: 2) alone, or each of these CAR variants in combination with the costimulatory polypeptides listed in Table 2 separated by a P2A ribosomal skip sequence. Transduced T cells were evaluated for their ability to proliferate and produce cytokines (IL-2) upon incubation with GPC3-expressing Hep 3B target cells. Transduced T cells T (effector) and Hep 3B cells (target) were plated at a 2:1 effector-to-target ratio (100,000 effector cells; 50,000 target cells) in RPMI 1640 supplemented with 10% heat inactivated fetal bovine serum (FBS), with re-stimulation every 3 to 4 days with 50,000 fresh target cells over a 14-day assay. The number of CD3-positive cells were evaluated by flow cytometry as a measure of T cell proliferation at each re-stimulation time point, and the area under the curve (AUC) of total CD3+ T cell counts was calculated from the plots of counts vs. time, using GraphPad Prism 7 version 7.0a for Mac OS X, GraphPad Software, La Jolla Calif. USA. Cytokine production (IL-2) was measured from the culture supernatants at 24 hours using the Meso Scale Discovery V-Plex Human IL-2 kit according to the manufacturer's protocol. Relative IL-2 concentrations and proliferation AUC values were calculated as a percent of values for control cognate GPC3 CAR variant (parent) without an additional costimulatory polypeptide within each assay as indicated in Table 2. In some instances, the activity of T cells expressing GPC3-CAR-CD28 in combination with costimulatory polypeptides was compared to that of T cells expressing GPC3-CAR-4-1BB. Co-expression of a costimulatory peptide was determined to enhance function if the activity was >115% that of its cognate parent for both IL-2 production and proliferation or >140% in at least one of these assays.

These experiments demonstrate that expressing tumor necrosis factor (TNF) superfamily costimulatory polypeptides or B7/CD28 superfamily costimulatory peptides in T cells in combination with an anti-GPC3 CAR can enhance the activity of the T cell relative to the anti-GPC3 CAR alone in the context of both anti-GPC3 CAR polypeptide with a 4-1BB costimulatory domain and an anti-GPC3 CAR polypeptide with a CD28 costimulatory domain, but not all costimulatory polypeptides enhance activity. Co-expressing a CD27 costimulatory polypeptide enhanced activity of T cells expressing the GPC3-CAR-4-1BB; co-expressing CD40L and TL1A costimulatory polypeptides enhanced activity of T cells expressing GPC3-CAR-CD28.

TABLE 2
Scoring of the in vitro proliferation and IL-2
release of variants co-expressing anti-GPC3
CAR and costimulatory polypeptides relative to
parent 4-1BB-containing or parent CD28-containing
CAR variant.

| Primary | | Relative | |
|---|---|---|---|
| costim- | | Activity | |
| ulatory | Costimulatory | | Prolif- |
| domain | polypeptide | IL-2 | eration |
| 4-1BB | — | 100% | 100% |
| | CD30L (SEQ ID NO: 36) | 62% | 82% |
| | CD40L (SEQ ID NO: 38) | 17% | 66% |
| | GITRL (SEQ ID NO: 41) | 48% | 76% |
| | ICOSL (SEQ ID NO: 16) | 71% | 86% |
| | TL1A (SEQ ID NO: 50) | 37% | 68% |
| | BAFFR (SEQ ID NO: 32) | 157% | 83% |
| | CD40 (SEQ ID NO: 37) | 77% | 88% |
| | CD27 (SEQ ID NO: 33) | 130% | 116% |
| | OX40 (SEQ ID NO: 46) | 103% | 69% |
| | ICOS (SEQ ID NO: 15) | 77% | 43% |
| | 4-1BB (SEQ ID NO: 22) | 45% | 52% |
| CD28 | — | 100% | 100% |
| | CD30L (SEQ ID NO: 36) | 100% | 108% |
| | CD40L (SEQ ID NO: 38) | 126% | 118% |
| | GITRL (SEQ ID NO: 41) | 87% | 109% |
| | ICOSL (SEQ ID NO: 16) | 88% | 88% |
| | TL1A (SEQ ID NO: 50) | 143% | 101% |
| | BAFFR (SEQ ID NO: 32)* | 465% | 55% |
| | CD40 (SEQ ID NO: 37)* | 224% | 97% |
| | OX40 (SEQ ID NO: 46)* | 325% | 40% |
| | ICOS (SEQ ID NO: 15)* | 243% | 43% |

*When co-expressed with GPC3-CAR-CD28, these variants were scored relative to the GPC3-CAR-4-1BB variant alone.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A genetically engineered hematopoietic cell, wherein the hematopoietic cell co-expresses:
(i) a chimeric antigen receptor (CAR) polypeptide; wherein the CAR polypeptide comprises:
(a) an extracellular antigen binding domain, wherein the extracellular binding domain is specific to glypican-3 (GPC3);
(b) a transmembrane domain; and
(c) a cytoplasmic signaling domain; and
(ii) a co-stimulatory polypeptide, wherein the co-stimulatory polypeptide is a member of the B7/CD28 superfamily, a member of the tumor necrosis factor (TNF) superfamily, or a ligand thereof, wherein the co-stimulatory polypeptide is encoded by an exogenous nucleic acid.

2. The hematopoietic cell of claim 1, wherein the co-stimulatory polypeptide is a member of the B7/CD28 superfamily or a ligand thereof, which is selected from the group consisting of CD28, CD80, CD86, ICOS, ICOSL, B7-H3, B7-H4, VISTA, TMIGD2, B7-H6, and B7-H7.

3. The hematopoietic cell of claim 1, wherein the co-stimulatory polypeptide is a member of the TNF superfamily or a ligand thereof, which is selected from the group consisting of 4-1BB, 4-1BBL, BAFF, BAFFR, CD27, CD70, CD30, CD30L, CD40, CD40L, DR3, GITR, GITRL, HVEM, LIGHT, TNF-beta, OX40, OX40L, RELT, TACI, TL1A, TNF-alpha, TNFRII, BCMA, EDAR2, TROY, LTBR, EDAR, NGFR, OPG, RANK, DCR3, TNFR1, FN14 (TweakR), APRIL, EDA-A2, TWEAK, LTb (TNF-C), NGF, EDA-A1, APP amyloid precursor protein (APP), and TRAIL.

4. The hematopoietic cell of claim 1, wherein the CAR polypeptide further comprises at least one co-stimulatory signaling domain.

5. The hematopoietic cell of claim 4, wherein the at least one co-stimulatory signaling domain is of a co-stimulatory molecule selected from the group consisting of 4-1BB, CD28, CD28_{LL→GG} variant, OX40, ICOS, CD27, GITR, ICOS, HVEM, TIM1, LFA1, and CD2.

6. The hematopoietic cell of claim 4, wherein:
(i) the CAR polypeptide comprises a co-stimulatory domain of a CD28 co-stimulatory molecule; and
(ii) the co-stimulatory polypeptide is BAFFR or CD27.

7. The hematopoietic cell of claim 6, wherein the CD28 co-stimulatory molecule comprises the amino acid sequence of SEQ ID NO: 12.

8. The hematopoietic cell of claim 4, wherein:
(i) the CAR polypeptide comprises a co-stimulatory domain of a 4-1BB co-stimulatory molecule; and
(ii) the co-stimulatory polypeptide is CD70, LIGHT, or OX40L.

9. The hematopoietic cell of claim 8, wherein the 4-1BB co-stimulatory molecule comprises the amino acid sequence of SEQ ID NO: 22.

10. The hematopoietic cell of claim 8, wherein the CD70 comprises the amino acid sequence of SEQ ID NO: 34, the LIGHT comprises the amino acid sequence of SEQ ID NO: 43, and the OX40L comprises the amino acid sequence of SEQ ID NO: 47.

11. The hematopoietic cell of claim 1, wherein the extracellular antigen binding domain of (a) is a single chain antibody fragment (scFv) that is specific to GPC3.

12. The hematopoietic cell of claim 11, wherein the scFv comprises a heavy chain variable region set forth as SEQ ID NO: 74 and a light chain variable region set forth as SEQ ID NO: 75.

13. The hematopoietic cell of claim 1, wherein the transmembrane domain of (b) is of a single-pass membrane protein.

14. The hematopoietic cell of claim 13, wherein the transmembrane domain is a membrane protein selected from the group consisting of CD8α, CD8β, 4-1BB, CD28, CD34, CD4, FcεRIγ, CD16A, OX40, CD3ζ, CD3ε, CD3γ, CD3δ, TCRα, CD32, CD64, VEGFR2, FAS, and FGFR2B.

15. The hematopoietic cell of claim 1, wherein the transmembrane domain of (b) is a non-naturally occurring hydrophobic protein segment.

16. The hematopoietic cell of claim 1, wherein the cytoplasmic signaling domain in (c) comprises an immunoreceptor tyrosine-based activation motif (ITAM).

17. The hematopoietic cell of claim 16, wherein the cytoplasmic signaling domain of (c) is a cytoplasmic domain of CD3ζ or FcεR1γ.

18. The hematopoietic cell of claim 1, wherein the CAR polypeptide further comprises a hinge domain, which is located at the C-terminus of (a) and the N-terminus of (b).

19. The hematopoietic cell of claim 18, wherein the hinge domain is of CD28, CD16A, CD8α, or IgG.

20. The hematopoietic cell of claim 18, wherein the hinge domain is a non-naturally occurring peptide.

21. The hematopoietic cell of claim 1, wherein the CAR polypeptide further comprises a signal peptide at its N-terminus.

22. The hematopoietic cell of claim 1, wherein the hematopoietic cell is a hematopoietic stem cell or an immune cell, optionally wherein the immune cell is a natural killer cell, macrophage, neutrophil, eosinophil, or T cell.

23. The hematopoietic cell of claim 22, wherein the immune cell is a T cell, in which the expression of an endogenous T cell receptor, an endogenous major histocompatibility complex, an endogenous beta-2-microglobulin, or a combination thereof has been inhibited or eliminated.

24. The hematopoietic cell of claim 1, wherein the hematopoietic cell is derived from peripheral blood mononuclear cells (PBMC), hematopoietic stem cells (HSCs), or inducible pluripotent stem cells (iPSCs).

25. The hematopoietic cell of claim 1, wherein the hematopoietic cell comprises a nucleic acid or nucleic acid set, which collectively comprises:
(A) a first exogenous nucleotide sequence encoding the co-stimulatory polypeptide; and
(B) a second exogenous nucleotide sequence encoding the CAR polypeptide.

26. The hematopoietic cell of claim 25, wherein the nucleic acid or the nucleic acid set is an RNA molecule or a set of RNA molecules.

27. The hematopoietic cell of claim 25, wherein the hematopoietic cell comprises the nucleic acid, which comprises both the first exogenous nucleotide sequence and the second exogenous nucleotide sequence.

28. The hematopoietic cell of claim 27, wherein the nucleic acid further comprises a third exogenous nucleotide sequence located between the first exogenous nucleotide sequence and the second exogenous nucleotide sequence, wherein the third exogenous nucleotide sequence encodes a ribosomal skipping site, an internal ribosome entry site (IRES), or a second promoter.

29. The hematopoietic cell of claim 30, wherein the third exogenous nucleotide sequence encodes a ribosomal skipping site, which is a P2A peptide.

30. The hematopoietic cell of claim 25, wherein the nucleic acid or the nucleic acid set is comprised within a vector or a set of vectors.

31. The hematopoietic cell of claim 30, wherein the vector or set of vectors is an expression vector or a set of expression vectors.

32. The hematopoietic cell of claim 30, wherein the vector or set of vectors comprises one or more viral vectors.

33. The hematopoietic cell of claim 32, wherein the one or more viral vectors is a retroviral vector, which optionally is a lentiviral vector or a gammaretroviral vector.

34. The hematopoietic cell of claim 25, wherein the nucleic acid or the nucleic acid set encoding the (i) CAR polypeptide; and (ii) the co-stimulatory polypeptide is delivered into the hematopoietic cell via transposons or gene editing.

35. A pharmaceutical composition, comprising a hematopoietic cell of claim 1, and a pharmaceutically acceptable carrier.

36. A method for inhibiting cells expressing GPC3 in a subject, the method comprising administering to a subject in need thereof a population of the hematopoietic cells set forth in claim 1 or a pharmaceutical composition comprising the hematopoietic cells.

37. The method of claim 36, wherein the hematopoietic cells are autologous.

38. The method of claim 36, wherein the hematopoietic cells are allogeneic.

39. The method of claim 37, wherein the hematopoietic cells are activated, expanded, or both ex vivo.

40. The method of claim 36, wherein the subject is a human patient suffering from a cancer associated with GPC3⁺ cancer cells.

41. The method of claim 40, wherein the cancer is breast cancer, gastric cancer, lung cancer, skin cancer, prostate cancer, colorectal cancer, renal cell carcinoma, ovarian cancer, rhabdomyosarcoma, germ cell cancer, hepatoblastoma, mesothelioma, pancreatic cancer, head and neck cancer, glioma, glioblastoma, thyroid cancer, hepatocellular cancer, esophageal cancer, or cervical cancer.

42. The method of claim 40, wherein the cancer is hepatocellular carcinoma, gastric cancer, breast cancer, or lung cancer.

43. The method of claim 36, wherein the hematopoietic cells are immune cells comprising T cells, which are activated in the presence of one or more of anti-CD3 antibody, anti-CD28 antibody, IL-2, phytohemoagglutinin, and an engineered artificial stimulatory cell or particle.

44. The method of claim 36, wherein the hematopoietic cells are immune cells comprising natural killer cells, which are activated in the presence of one or more of 4-1BB ligand, anti-4-1BB antibody, IL-15, anti-IL-15 receptor antibody, IL-2, IL-12, IL-18, IL-21 and K562 cells.

45. The method of claim 40, wherein the human patient has been treated or is undergoing an anti-cancer therapy.

46. The method of claim 40, further comprising administering to the subject an anti-cancer agent.

47. A nucleic acid or nucleic acid set, which collectively comprises:
(A) a first nucleotide sequence encoding a CAR polypeptide set forth in claim 1; and
(B) a second nucleotide sequence encoding a co-stimulatory polypeptide set forth in claim 1.

48. The nucleic acid or nucleic acid set of claim 47, wherein the nucleic acid or the nucleic acid set is an RNA molecule or a set of RNA molecules.

49. The nucleic acid or nucleic acid set of claim 47, wherein the nucleic acid comprises both the first nucleotide sequence and the second nucleotide sequence, and wherein the nucleic acid further comprises a third nucleotide sequence located between the first nucleotide sequence and the second nucleotide sequence, the third nucleotide sequence encoding a ribosomal skipping site, an internal ribosome entry site (IRES), or a second promoter.

50. The nucleic acid or nucleic acid set of claim 49, wherein the ribosomal skipping site is a P2A peptide.

51. The nucleic acid or nucleic acid set of claim 47, wherein the nucleic acid or the nucleic acid set is comprised within a vector or a set of vectors.

52. The nucleic acid or nucleic acid set of claim 51, wherein the vector or set of vectors is an expression vector or a set of expression vectors.

53. The nucleic acid or nucleic acid set of claim 51, wherein the vector or set of vectors comprises one or more viral vectors.

54. The nucleic acid or nucleic acid set of claim 53, wherein the one or more viral vectors is a retroviral vector, which optionally is a lentiviral vector or a gammaretroviral vector.

55. A method for generating modified hematopoietic cells in vivo, the method comprising administering to a subject in need thereof the nucleic acid or nucleic acid set of claim 47.


SEQUENCE LISTING (75 sequences)

SEQ ID NO: 1 - protein, 487 aa, Artificial Sequence
  Synthetic
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 483 aa, Artificial Sequence
  Synthetic
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 300 aa, Artificial Sequence
  Synthetic
  MISC_FEATURE (16)..(300): may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 4 - protein, 15 aa, Artificial Sequence
  Synthetic
GGGGSGGGGSGGGGS

SEQ ID NO: 5 - protein, 30 aa, Artificial Sequence
  Synthetic
GGGGSGGGGSGGGGSGGGGSGGGGSGGGGS

SEQ ID NO: 6 - protein, 45 aa, Artificial Sequence
  Synthetic
GGGGSGGGGSGGGGSGGGGSGGGGSGGGGSGGGGSGGGGSGGGGS

SEQ ID NO: 7 - protein, 60 aa, Artificial Sequence
  Synthetic
GGGGSGGGGSGGGGSGGGGSGGGGSGGGGSGGGGSGGGGSGGGGSGGGGSGGGGSGGGGS

SEQ ID NO: 8 - protein, 75 aa, Artificial Sequence
  Synthetic
GGGGSGGGGSGGGGSGGGGSGGGGSGGGGSGGGGSGGGGSGGGGSGGGGSGGGGSGGGGSGGGGSGGGGSGGGGS

SEQ ID NO: 9 - protein, 150 aa, Artificial Sequence
  Synthetic
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 10 - protein, 225 aa, Artificial Sequence
  Synthetic
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 11 - protein, 300 aa, Artificial Sequence; identical to SEQ ID NO: 3
  Synthetic

SEQ ID NO: 12 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 13 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 14 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 15 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 16 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 17 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 18 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 19 - protein, 282 aa, Homo sapiens
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 20 - protein, 454 aa, Homo sapiens
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 21 - protein, 414 aa, Homo sapiens
MKAQTALSFFLILITSLSGSQGIFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 22 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 23 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 24 - protein, 231 aa, Homo sapiens
MRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 25 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRAGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 26 - protein, 231 aa, Homo sapiens
MRVLPWALVAGLLLLLLLAAACAVFLACPWAVSGARASPGSAASPRLREGPELSPDDPAGLLDLRAGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 27 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGAAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 28 - protein, 231 aa, Homo sapiens
MRVLPWALVAGLLLLLLLAAACAVFLACPWAVSGARASPGSAASPRLREGPELSPDDPAGLLDLRQGMFAQLVAQNVLLIDGPLSWYSDPGAAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 29 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYAEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 30 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWALTQGATVLGLFRVTPEIPAGLPSPRSE

SEQ ID NO: 31 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 32 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 33 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 34 - protein, 193 aa, Homo sapiens [sequence omitted]

SEQ ID NO: 35 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 36 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 37 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 38 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 39 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 40 - protein, 240 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLW

SEQ ID NO: 41 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 42 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 43 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 44 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 45 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 46 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 47 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 48 - protein, 430 aa, Homo sapiens
MKPSLLCRPLSCFLMLLPWPLATLTSTTLWQCPPGEEPDLDPGQGTLCRPCPPGTFSAAWGSSPCQPHARCSLWRRLEAQVGMATRDTLCGDCWPGWFGPWGVPRVPCQPCSWAPLGTHGCDEWGRRARRGVEVAAGASSGGETRQPGNGTRAGGPEETAAQYAVIAIVPVFCLMGLLGILVCNLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 49 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 50 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 51 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 52 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 53 - protein, 297 aa, Homo sapiens
MDCQENEYWDQWGRCVTCQRCGPGQELSKDCGYGEGGDAYCTACPPRRYKSSWGHHRCQSCITCAVINRVQKVNCTATSNAVCGDCLPRFYRKTRIGGLQDQECIPCTKQTPTSEVQCAFQLSLVEADTPTVPPQEATLVALVSSLLVVFTLAFLGLFFLYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP

SEQ ID NO: 54 - protein, 423 aa, Homo sapiens
MALKVLLEQEKTFFTLLVLLGYLSCKVTCESGDCRQQEFRDRSGNCVPCNQCGPGMELSKECGFGYGEDAQCVTCRLHRFKEDWGFQKCKPCLDCAVVNRFQKANCSATSDAICGDCLPGFYRKTKLVGFQDMECVPCGDPPPPYEPHCASKVNLVKIASTASSPRDTALAAVICSALATVLLALLILCVIYCKRQFMEKKPSWSLRSQDIQYNGSELSCFDRPQLHEYAHRACCQCRRDSVQTCGPVRLLPSMCCEEACSPNPATLGCGVHSAASLQARNAGPAGEMVPTFFGSLTQSICGEFSDAWPLMQNPMGGDNISFCDSYPELTGEDIHSLNPELESSTSLDSNSSQDLVGGAVPVQSHSENFTAATDLSRYNNTLVESASTQDALTMRSQLDQESGAVIHPATQTSLQVRQRLGSL

SEQ ID NO: 55 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 56 - protein, 448 aa, Homo sapiens
MAHVGDCTQTPWLPVLVVSLMCSARAEYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATALIIAMSTIFIMAIAIVLIIMFYILKTKPSAPACCTSHPGKSVEAQVSKDEEKKEAPDNVVMFSEKDEFEKLTATPAKPTKSENDASSENEQLLSRSVDSDEEPAPDKQGSPELCLLSLVHLAREKSATSNKSAGIQSRRKKILDVYANVCGVVEGLSPTELPFDCLEKTSRMLSSTYNSEKAVVKTWRHLAESFGLKRDEIGGMTDGMQLFDRISTAGYSIPELLTKLVQIERLDAVESLCADILEWAGVVPPASQPHAAS

SEQ ID NO: 57 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 58 - protein, 401 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 59 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 60 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 61 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 62 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 63 - protein, 250 aa, Homo sapiens [sequence omitted]

SEQ ID NO: 64 - protein, 389 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVYYINFTDFASYEVVVDEKPFLQCTRSIETGKTNYNTCYTAGVCLLKARQKIAVKMVHADISINMSKHTTFFGAIRLGEAPAS

SEQ ID NO: 65 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 66 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 67 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 68 - protein, 391 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 69 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 70 - protein, 281 aa, Homo sapiens [sequence omitted]

SEQ ID NO: 71 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 72 - protein, 19 aa, Artificial Sequence
  Synthetic
ATNFSLLKQAGDVEENPGP

SEQ ID NO: 73 - protein, 18 aa, Artificial Sequence
  Synthetic
ATNFSLLKQAGDVEENPG

SEQ ID NO: 74 - protein, 111 aa, Artificial Sequence
  Synthetic
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLV

SEQ ID NO: 75 - protein, 111 aa, Artificial Sequence
  Synthetic
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEI